(19) United States
(12) Patent Application Publication
(10) Pub. No.: US 2022/0313658 A1
(43) Pub. Date: Oct. 6, 2022

(54) YAP Inhibition for Wound Healing

(71) Applicant: The Board of Trustees of the Leland Stanford Junior University, Stanford, CA (US)

(72) Inventors: Shamik Mascharak, Palo Alto, CA (US); Heather Elizabeth DesJardins-Park, Stanford, CA (US); Mimi Borrelli, Stanford, CA (US); Michael Francis Davitt, Stanford, CA (US); Michael T. Longaker, Stanford, CA (US)

(21) Appl. No.: 17/626,699

(22) PCT Filed: Jul. 24, 2020
(86) PCT No.: PCT/US2020/043420

§ 371 Date: Jan. 12, 2022

Related U.S. Application Data
(60) Provisional application No. 62/879,369, filed on Jul. 26, 2019.

(51) Int. Cl. A61K 31/409; A61P 17/02; A61P 17/14

(52) U.S. Cl. CPC A61K 31/409; A61P 17/02; A61P 17/14

(57) ABSTRACT

Methods of promoting healing of a wound in a dermal location of a subject are provided. Aspects of the methods may include administering an effective amount of a YAP inhibitor composition to the wound to modulate mechanical activation of Engrailed-1 lineage-negative fibroblasts (ENFs) in the wound to promote ENF-mediated healing of the wound. Also provided are methods of preventing scarring during healing of a wound in a subject and methods of promoting hair growth on a subject. Aspects of the methods may include forming a wound in a dermal location of a subject and administering an effective amount of a YAP inhibitor composition to the wound to modulate mechanical activation of Engrailed-1 lineage-negative fibroblasts (ENFs) in the wound to promote ENF-mediated healing of the wound. Also provided are kits including an amount of a YAP inhibitor composition and a tissue disrupting device.

### CROSS-REFERENCE TO RELATED APPLICATIONS

This application is a U.S. National Stage Application of PCT Application No. PCT/US2020/043420 filed Jul. 24, 2020, which application, pursuant to 35 U.S.C. § 119(e), claims priority to the filing date of the U.S. Provisional Patent Application Ser. No. 62/879,369 filed Jul. 26, 2019; the disclosures of which applications are herein incorporated by reference.

### ACKNOWLEDGEMENT OF GOVERNMENT RIGHTS

This invention was made with Government support under contract GM116892 awarded by the National Institutes of Health. The Government has certain rights in this invention.

### INTRODUCTION

The skin is the largest organ in the body consisting of several layers and plays an important role in biologic homeostasis. The skin has multiple functions, including thermal regulation, metabolic function (vitamin D metabolism), and immune functions. Mammalian skin includes two main layers, the epidermis and the dermis. The epidermis is outermost layer of skin and serves as a protective barrier to the environment. The dermis is the layer of skin beneath the epidermis and serves a location for the appendages of skin including, e.g., hair follicles, sweat glands, sebaceous glands, apocrine glands, lymphatic vessels and blood vessels. The dermis provides strength and elasticity to the skin through an extracellular matrix or connective tissue made of structural proteins (collagen and elastin), specialized proteins (fibrillin, fibronectin, and laminin), and proteoglycans. The epidermis and dermis are separated by the basement membrane, a thin, fibrous extracellular matrix.

Hair is a protein filament that grows from hair follicles present in the dermis. Hair is a primary differentiator of mammals from other classes of organisms. Hair may protect from cold and UV radiation, shield organs from dirt and sweat, and provide a sensory function. Each hair is made up of two separate structures: the hair shaft and the follicle. The hair shaft includes the visible part outside of the skin. The hair follicle is an organ from which hair can grow and regulates hair growth via a complex interaction between hormones, neuropeptides and immune cells. The histological arrangement of the follicle is divided into outer and inner root sheaths. Hair loss is an extremely common issue affecting billions of individuals worldwide. For example, androgenetic alopecia, or male pattern hair loss, is estimated to impact over 90% of men by age 50 and over 50% of women by age 65. Hair loss can occur as a result of skin scarring (e.g., following mechanical injury or burns) or autoimmune conditions (e.g., alopecia areata).

Wound healing or tissue healing is a biological process that involves tissue regeneration. During the process of healing, damaged or destroyed tissue is replaced with living tissue. When the skin barrier is broken, a regulated sequence of biochemical events is activated to repair the damage. The process is regulated by numerous biological components including, e.g., growth factors, cytokines, and chemokines, and employs several components including, e.g., soluble mediators, blood cells, extracellular matrix components, and parenchymal cells. Wound healing generally proceeds through several stages. The process is divided into several phases including hemostasis, inflammation, proliferation, and remodeling. The end point of wound healing may include the formation of a scar. Skin wounds invariably heal by developing fibrotic scar tissue, which can result in disfigurement, growth restriction, and permanent functional loss. Various types of scars may form after skin tissue repair including, e.g., a “normal” fine line and abnormal scars including widespread scars, atrophic scars, scar contractures, hypertrophic scars, and keloid scars.

### SUMMARY

No current therapeutic strategies exist for successfully preventing or reversing the fibrotic process that leads to scarring. Attempts at reducing scarring often entail ablation of cell populations known to be fibrogenic, but this approach could impair or delay wound repair by nonspecifically eliminating cells that are needed for proper healing. Skin regeneration as defined by recovery of three features of normal skin: 1) secondary elements (e.g., dermal appendages), 2) ECM structure, and 3) mechanical strength—has not been achieved.

In addition, no effective therapies for restoring the hair-growing potential of skin exists. In particular, no targeted molecular agents have proven capable of inducing hair follicle regeneration. The most effective existing treatments generally involve grafting hair-growing skin into areas affected by alopecia, an approach that is limited by availability of graftable tissue, donor site morbidity, and cost. No therapeutic strategies exist that successfully promote regeneration of new, endogenous hair follicles in areas affected by hair loss.

Methods of promoting healing of a wound in a dermal location of a subject are provided. Aspects of the methods may include administering an effective amount of a YAP inhibitor composition to the wound to modulate mechanical activation of Engrailed- 1 lineage-negative fibroblasts (ENFs) in the wound to promote ENF-mediated healing of the wound. Also provided are methods of preventing scarring during healing of a wound in a subject and methods of promoting hair growth on a subject. Aspects of the methods may include forming a wound in a dermal location of a subject and administering an effective amount of a YAP inhibitor composition to the wound to modulate mechanical activation of Engrailed-1 lineage-negative fibroblasts (ENFs) in the wound to promote ENF-mediated healing of the wound. Also provided are kits including an amount of a YAP inhibitor composition and a tissue disrupting device.

### BRIEF DESCRIPTION OF THE FIGURES

AG. 1, A-1 illustrates deep dermal ENFs activate Engrailed-1 and contribute to postnatal scar collagen deposition. (A) Schematic depicting cell transplantation, engraftment, and wounding experiments. (B) Fluorescent imaging of Engrailed-i-positive fibroblasts (EPFs, left column) and Engrailed-1-negative fibroblasts (ENFs, right column) following transplantation into unwounded skin (top row) or transplantation followed by excisional wounding (bottom row). (C) Histology of ENFs (red) subjected to transplantation and wounding, with postnatal EPFs (pEPFs, green) derived from conversion of ENFs to EPFs within the wound following transplantation; immunostaining for type I collagen (col-I) shown in white. Top, merged; bottom left, ENFs and EPFs; bottom right, col-I staining. N=3 mice each receiving ENFs and EPFs, 2 wounds/mouse. (D) Top: 3D reconstruction of confocal imaging shown in (C), generated using Imaris software (ENFs, red; pEPFs, green; col-I, white). Bottom: Quantification of signal colocalization between col-I staining and either Tomato (ENF) or GFP (pEPF) signal. Points represent averages per wound. N=5-6 wounds, *P=0.0335. (E) Schematic depicting tamoxifen induction followed by wounding of En1^{Cre-ERT}; Ai/6 mice for temporally-defined assessment of En-1 activation during wound healing. (F) Histology of unwounded skin (top row) and healed wounds (POD 14; bottom row) from tamoxifen-induced En1^{Cre-ERT}; Ai6 mice, where GFP⁺ cells (EPFs, green) necessarily arose from En-1 expression activated during wound healing (white arrows). Immunostaining for Dlk-1 (red) and col-I (white); DAPI, blue. N=4 mice, 2 wounds/mice. (G) Proposed mechanism for postnatal En-1 activation. ENFs (red) resident in the dermis (top), when exposed to wound-specific cues, give rise to pEPFs (red-to-green cells; middle). These pEPFs, along with embryonically-derived EPFs (eEPFs), mediate scarring wound repair (bottom). (H) Schematic depicting isolation of three ENF subtypes and separate transplantation followed by wounding for each subtype. (I) Transplantation and wounding of papillary (CD26⁺, left), reticular (Dlk1⁺ Scat, middle), and hypodermal (Dlk1^{-/-} Sca1⁺, right) ENFs (white) into an mTomato-expressing recipient mouse (red) shows that only reticular ENFs give rise to pEPFs (green, white arrows). DAPI, blue. N=3 mice receiving each ENF subtype, 1 wound/mouse.

FIG. 2, A-I illustrates reticular dermal ENFs activate Engrailed-1 via canonical mechanotransduction signaling in response to in vitro and in vivo substrate mechanics. (A)

Isolation and culture of ENFs on substrates with varying mechanics: stiff plastic (with or without ROCK inhibitor Y-27632; top) or soft hydrogel (bottom). (B) ENFs after one (top row) or 14 (bottom row) days in culture on stiff TCPS (left column), Tops with ROCK inhibitor (Y-27632; middle column), or soft hydrogel (right column) showing variable conversion of ENFs (red) to pEPFs (green). (C) Quantification of percentage of ENFs converted to EPFs over time in culture on different substrates. N=3 experimental replicates using P1 ENFs derived from separate litters. (D) Schematic depicting fractionation and culture of ENF subpopulations on stiff substrate (TOPS) with or without ROCK inhibitor (Y-27632). (E) Papillary (left column), reticular (middle column), and hypodermal (right column) ENFs after 14 days of culture on TOPS, with (bottom row) or without (top row) mechanotransduction inhibition, showing En-1 activation (GFP, green) only in reticular dermal ENFs on TOPS (top row, middle panel). N=3 experimental replicates using P1 ENFs derived from separate litters. (F) Schematic of canonical mechanotransduction signaling pathway. Mechanical forces are signaled through activation of FAK and downstream Rho and ROCK; Verteporfin inhibits mechanotransduction by inhibiting YAP, the pathways final transcriptional effector. (G) Left panel: Schematic depicting the strategy for applying tension over dorsal wounds. Right panels: Gross photographs of healed dorsal incisional wounds following control sham (left photo), application of increased tension (middle photo), or increased tension and Verteporfin treatment (right photo). (H) Fluorescent histology of control (left column), tension-treated (middle column), and tension- and Verteporfin-treated (right column) wounds in En-1^{Cre-ERT}; Ai6 mice showing increased pEPFs (green) with increased tension, Immunofluorescent staining for a-SMA (red) and YAP (white); DAPI, blue. Bottom rows, individual channels; top row, merged. (I) Quantification of GFP+ cells (pEPFs; top panel) and YAP+ cells (bottom panel) per 20× high-powered field (HPF). (G-I) N=4-5 mice/condition.

FIG 3, A-L illustrates mechanical activation of DIM+ ENFs is associated with a fibrotic transcriptional signature. (A) Schematic of bulk ENFs cultured in vitro for 2, 7, or 14 days. (B) Gene expression heatmap and hierarchical clustering for 920 genes significantly upregulated (>4-fold) or downregulated (<1/4-fold) at day 14 in culture compared to day 2. Values shown for 2, 7, or 14 days in culture, or 14 days in culture with Verteporfin (Vert) treatment (purple box) (labels at bottom of plot). (C) Volcano plot of 920 differentially expressed genes (day 14 vs. 2) depicted in (B). (D) Principal component analysis (PCA) of RNA-seq data from cultured ENFs at different timepoints, with and without Vert treatment. Clusters for each timepoint and condition are indicated by ovals. (E) GO term enrichments for significantly upregulated (top plot) or downregulated (bottom plot) genes depicted in (B), for ENFs at 14 days in culture with or without Vert. (F) Heatmap showing relative expression of selected genes previously implicated in fibrosis and ECM deposition. Dlk1 was upregulated in ENFs at 7 days (red box). Pro-fibrotic/matrix genes were largely upregulated at 14 days (green box); these changes were mitigated with Vert treatment (purple box). N=2 biological replicates per experimental group (pooled ENFs from 2 separate litters, 10 pups each). (G) Schematic depicting isolation of scar pEPFs and scar and unwounded skin eEPFs and ENFs for RNA-seq. (H) Heatmap and hierarchical clustering of 1,138 genes significantly upregulated or downregulated in ENFs, eEPFs, or pEPFs in wounds (inj) compared to uninjured skin (uninj). (I) Volcano plot showing 1,138 differentially expressed genes depicted in (H). Individual plots are labeled (top right corner) with comparisons shown in each plot. (J) RCA of RNA-seq data for pEPFs, eEPFs, and ENFs from injured and uninjured skin. (K) Comparison of Dpp4 (CD26; left panel), Jag1 (middle panel), and DIll (right panel) gene counts for each cell type. (L) Heatmaps showing relative expression of selected genes previously reported to be associated with ENF (left panel) or EPF (right panel) identity. N=2 biological replicates per experimental group (24 scars and 6 unwounded skin pieces from 6 mice pooled into 2 groups each). Green boxes, EPF populations (pEPF, inj and uninj eEPF); red boxes, ENFs (inj and uninj ENF).

FIG. 4, A-H illustrates mechanotransduction inhibition in vivo results in starless wound healing via regeneration. (A) Schematic of dorsal excisional wounding (top row), with corresponding gross photographs for each timepoint of wounds treated with PBS (control; middle row) or Verteporfin (bottom row), at POD 0 (left column), 14 (middle left column), 30 (middle right column), and 90 (right column). Red dotted circles indicate location of rings used to splint wounds. (B) H&E histology of control- (top row) and Verteporfin-treated (bottom row) wounds harvested at POD 14 (left column), 30 (middle column), or 90 (right column). White arrows indicate structures morphologically consistent with dermal appendages. (C) Verteporfin-treated wound at POD 90 demonstrating regrowth of hair follicles and other dermal appendages. Gross photograph (top row) and histology: middle row, immunostaining for hair follicle/sweat gland markers CK14 (red) and CK19 (green) (DAPI, blue); bottom row, Oil Red O staining (red) for sebaceous glands. (D-F) Fluorescent histology of control- (top row) and Verteporfin-treated (bottom row) wounds at POD 14 (D), 30 (E), and 90 (F), showing fibroblasts (EPF, ENF) and immunostaining for ECM proteins (col-I, Fn) and fibroblast/mechanotransduction markers (CD26, Dlk-1, YAP, aSMA); colors indicated by labels in each panel. For panels (B-F), N=3 mice per condition/timepoint, 2 wounds/mouse. (F) Far right panel, quantification of GFP+ cells (EPFs) per 20× HPF for PBS- and Verteporfin-treated wounds after 2 weeks, 1 month, and 3 months of healing. (G) t-SNE plots visualizing 26 ECM ultrastructural properties for unwounded skin (green) and PBS- (red) or Verteporfin-treated (blue) wounds at POD 14 (i), 30 (ii), and 90 (iii), with clusters for each group highlighted by shaded regions. N=3 mice/condition, 5-10 images/mouse. Points represent single images. (H) Instron mechanical strength testing of unwounded skin (green), PBS- (red), and Verteporfin-treated (blue) wounds with calculated wound breaking force (left plot; unwounded vs. PBS, *P=0.0417; unwounded vs. Verteporfin, P=0.8057) and Young's modulus (right plot; unwounded vs. PBS. *P=0.0048; unwounded vs. Verteporfin, P=0.9287). Points represent individual mice. N=7 mice (unwounded), 5 mice (PBS), 4 mice (Verteporfin).

FIG. 5, A-F illustrates FACS strategies to isolate fibroblast subtypes. (A) Strategy for isolating ENFs (Lin⁻GFP⁻CD26⁻), eEPFs (Lin⁻GFP⁻CD26⁺), and pEPFs (Lin⁻GFP⁺) from tamoxifen-induced En-1^{Cre-ERT}; Ai6 dorsal skin and excisional wounds. (B) Representative FACS plots for unwounded skin (left) and wounds (right) depicted in (A). *, ** indicate gated cell populations carried over into subsequent plots. (C) Quantification of relative proportion of fibroblasts (Lin⁻) represented by ENFs (red), eEPFs (blue), and pEPFs (green) in unwounded skin vs. healed wounds (POD 14). Points represent biological replicates; N=3 biological replicates, each containing pooled cells from 4 mice (2 wounds/mouse). Unwounded vs, wounded: eEPFs, *P=0.0559; pEPFs, *P=0.0204; ENFs, P=0.6433. (D) Schematic for FACS isolation of papillary, reticular, and hypodermal fibroblasts from En-1^{Cre}; Ai6 dorsal skin based on previously reported surface markers. (E) Representative FACS plots showing gating strategy for isolating ENFs (Lin⁻GFP⁻; red box) and EPFs (Lin⁻GFP⁺; green box), and fractionation of ENF subtypes (papillary, blue box; reticular, gray box; hypodermal, purple box). *,**,***, and ‡ indicate gated cell populations carried over into subsequent plots. (F) Proportion of fibroblasts represented by each ENF subpopulation (papillary, blue; reticular, gray; hypodermal, purple) when fibroblasts are defined as PDGFRa⁺ cells (left panel) versus Lin⁻ cells (right panel). N=3 separate experiments using pooled cells from individual litters. Left; papillary vs. hypodermal *P=0.0135, reticular vs. hypodermal *P=0.0067. Right: all pairwise comparisons P>0.05.

FIG. 6, A-C illustrates gene set enrichment analysis for in vitro ENFs and pEPFs. Normalized RNA-seq counts for ENFs (mTomato⁺) cultured on TCPS for 2 days (remain as ENFs) or 14 days (activate Engraiied-1; GFP⁺) were analyzed for enrichment in the (A) Gene Ontology Biological Process, (B) Gene Ontology Molecular Function, and (C) Hallmark databases. Activation of Engrailed-1 was associated with the loss of “muscle development” identity and the gain of a pro-fibrotic identity, as inferred by enrichment for a variety of ECM-related terms at 14 days.

FIG. 7, A-C illustrates gene set enrichment analysis for in viva ENFs and pEPFs. Normalized RNA-seq counts for scar ENFs (GFP⁻CD26⁻) and postnatal EPFs (GFP⁻) were analyzed for enrichment in the (A) Gene Ontology Biological Process, (B) Gene Ontology Molecular Function, and (C) Hallmark databases. Scar ENFs were enriched for ECM-adhesion and Notch signaling-related terms, supporting their mechanosensitive phenotype. In contrast, postnatal EPFs were enriched for a variety of ECM-related terms, confirming that activation of Engrailed-1 in the wound environment by mechanosensitive ENFs was associated with the acquisition of a pro-fibrotic phenotype.

FIG. 8, A-C illustrates characterization of wounds treated with multiple doses of Verteporfin. (A) Wound curve showing closure (re-epithelialization) rates for wounds treated with PBS (red) versus 1 (blue), 2 (purple), or 4 (light blue) doses of Verteporfin at indicated intervals. N=at least 6 wounds/condition. POD 4, 2 dose Verteporfin vs. PBS, *P=0.0140; POD 8, 4 dose Verteporfin vs. PBS, *P=0.0140; all other comparisons, P>0.05. (B) Representative gross photographs of wounds treated with PBS (first row), 1 (second row), 2 (third row), or 4 (fourth row) doses of Verteporfin at POD 0 (left column) and 30 (right column). (C) t-SNE visualization of ECM ultrastructural properties for various treatment groups after 2 weeks or 1 month of healing (see legend). Clusters for unwounded skin and scar (PBS) highlighted by shaded regions.

FIG. 9, A-B illustrates quantification of ECM fiber parameters at 2 weeks following wounding. (A) Quantified fiber parameters from unwounded skin and Verteporfin- or PBS-treated wounds at POD 14. Separate values were calculated for mature (red) versus immature (green) fibers, as assessed by Picrosirius staining. Dots represent the average of two wounds from each of N=3 mice. (B) P-values for comparison of fiber parameters (red, mature; green, immature) between unwounded skin and either PBS- (left) or Verteporfin-treated wounds (right).

FIG. 10, A-B illustrates quantification of ECM fiber parameters at 1 month following wounding. (A) Quantified fiber parameters from unwounded skin and Verteporfin- or PBS-treated wounds at POD 30. Separate values were calculated for mature (red) versus immature (green) fibers, as assessed by Picrosirius staining. Dots represent the average of two wounds from each of N=3 mice. (B) P-values for comparison of fiber parameters (red, mature; green, immature) between unwounded skin and either PBS- (left) or Verteporfin-treated wounds (right).

FIG. 11, A-B illustrates quantification of ECM fiber parameters at 3 months following wounding. (A) Quantified fiber parameters from unwounded skin and Verteporfin- or PBS-treated wounds at POD 90. Separate values were calculated for mature (red) versus immature (green) fibers, as assessed by Picrosirius staining. Dots represent the average of two wounds from each of N=3 mice. (B) P-values for comparison of fiber parameters (red, mature; green, immature) between unwounded skin and either PBS- (left) or Verteporfin-treated wounds (right).

FIG. 12, A-B illustrates instron comparison of PBS- and Verteporfin-treated wounds after 1 month of healing. (A) Representative force-displacement curve for unwounded skin (green), PBS-treated wounds (red), and Verteporfin-treated wounds (blue) after 1 month of healing. (B) Representative stress-strain curve for the same groups as (A). Verteporfin treatment yielded wounds that more closely resembled unwounded skin than scar (PBS treatment) after 1 month of healing.

FIG. 13, A-C illustrates generation of new hair follicles in verteporfin-treated wounds. (A) Schematic of dorsal excisional wounding (top row), with corresponding gross photographs for each timepoint of wounds treated with PBS (control; middle row) or Verteporfin (bottom row), at POD 0 (left column), 14 (middle left column), 30 (middle right column), and 90 (right column). Red dotted circles indicate location of rings used to splint wounds. (B) H&E histology of control-(top row) and Verteporfin-treated (bottom row) wounds harvested at POD 14 (left column), 30 (middle column), or 90 (right column). White arrows indicate structures morphologically consistent with dermal appendages. (C) Verteporfin-treated wound at POD 90 demonstrating regrowth of hair follicles and other dermal appendages. Gross photograph (top row) and histology: bottom row, immunostaining for hair follicle/sweat gland markers CK14 (red) and CK19 (green) (DAPI, blue).

### DEFINITIONS

As used herein in its conventional sense, the term “fibroblast” refers to a cell responsible for synthesizing and organizing extracellular matrix. Two fibroblast lineages include Engrailed-1 lineage-negative fibroblasts (ENFs) and Engrailed-1 lineage-positive fibroblasts (EPFs). The EPF lineage includes all cells that express Engrailed-1 at any point during their development, and all progeny of those cells,

As used herein, the term “modulating” means increasing, reducing or inhibiting an attribute of a biological cell, population of cells, or a component of a cell (e.g., a protein, nucleic acid, etc.). In some cases, the attribute includes, e.g., activation of a signaling pathway. In some cases, the attribute includes an amount and/or activity of one or more cells. In some cases, the attribute includes, e.g., an amount, activity, or expression level (DNA or RNA expression levels) of a component of a cell (e.g., a protein, nucleic acid, etc.). In some cases, “modulate” or “modulating” or “modulation” may be measured using an appropriate in vitro assay, cellular assay or in vivo assay. In some cases, the increase or decrease is 10% or more relative to a reference, e.g., 10% or more, 20% or more, 30% or more, 40% or more, 50% or more, 60% or more, 70% or more, 80% or more, 90% or more, 95% or more, 97% or more, 98% or more, up to 100% relative to a reference. For example, the increase or decrease may be 2 or more times, 3 times or more, 4 times or more, 5 times or more, 6 times or more, 7 times or more, 8 times or more, 9 times or more, 10 times or more, 50 times or more, or 100 times or more relative to a reference.

The term “fibrosis” as used herein in its conventional sense refers to the formation or development of excess fibrous connective tissue in an organ or tissue as a result of injury or inflammation of a part or interference with its blood supply. It can be a consequence of the normal healing response that leads to a scar, an abnormal reactive process or no known or understood cause.

As used herein in its conventional sense, the term “scarring” refers to a condition in which fibrous tissue replaces normal tissue destroyed by injury or disease. The term “scarring” further refers to abnormality in one or more of color, contour (bulging/indentation), rugosity (roughness/smoothness) and texture (softness/hardness), arising during the skin healing process. The expression “preventing” or “prevent” used herein in the context of scarring refers to an adjustment to the extent of development of scarring, whereby one or more of the color, contour, rugosity and texture of the healed skin surface approximates on ordinary visual inspection to that of the subject's normal skin. The expression “reducing” or “reduce” used herein in the context of scarring refers to an adjustment to the extent of development of scarring, whereby one or more of the color, contour, rugosity and texture of the healed skin surface approaches measurably closer to that of the patient's normal skin.

As used herein in its conventional sense, the term “scar” refers to a fibrous tissue that replaces normal tissue destroyed by injury or disease. Damage to the outer layer of skin is healed by rebuilding the tissue, and in these instances, scarring is slight. When the thick layer of tissue beneath the skin is damaged, however, rebuilding is more complicated. The body lays down collagen fibers (a protein which is naturally produced by the body), and this usually results in a noticeable scar. After the wound has healed, the scar continues to alter as new collagen is formed and the blood vessels return to normal, allowing most scars to fade and improve in appearance over the two years following an injury. However, there is some visible evidence of the injury, and hair follicles and sweat glands do not grow back. As used herein, the term “scar area” refers to the area of normal tissue that is destroyed by injury or disease and replaced by fibrous tissue.

Scars differ from normal skin in three key ways: (1) they are devoid of any dermal appendages (hair follicles, sweat glands, etc.); (2) their collagen structure is fundamentally different, with dense, parallel fibers rather than the “basketweave” pattern that lends normal skin its flexibility and strength; and (3) as a result of their inferior matrix structure, they are weaker than skin.

The term “scar-related gene” as used herein refers to a nucleic acid encoding a protein that is activated in response to scarring as part of the normal wound healing process. The term “scar-related gene product” as used herein refers to the protein that is expressed in response to scarring as part of the normal wound healing process,

Scar tissue consists mainly of disorganized collagenous extracellular matrix. This is produced by myofibroblasts, which differentiate from dermal fibroblasts in response to wounding, which causes a rise in the local concentration of Transforming Growth Factor-β, a secreted protein that exists in at least three isoforms called TGF-βI, TGF-P2 and TGF-P3 (referred to collectively as TGF-β). TGF-β is an important cytokine associated with fibrosis in many tissue types (Beanes, S. et al, Expert Reviews in Molecular Medicine, vol. 5, no. 8, pp. 1-22 (2003)). Types of scars are further described in, e.g., PCT Application No. WO 2014/040074, the disclosure of which is incorporated herein by reference in its entirety.

The term “skin” used herein in its conventional sense includes all surface tissues of the body and sub-surface structure thereat including, e.g., mucosal membranes and eye tissue as well as ordinary skin. The expression “skin” may include a wound zone itself. The re-approximation of skin over the surface of a wound has long been a primary sign of the completion of a significant portion of wound healing. This reclosure of the defect restores the protective function of the skin, which includes protection from bacteria, toxins, and mechanical forces, as well as providing the barrier to retain essential body fluids. The epidermis, which is composed of several layers beginning with the stratum corneum, is the outermost layer of the skin, The innermost skin layer is the deep dermis.

As used herein in its conventional sense, the term “dermal appendages” includes hair follicles, sebaceous and sweat glands, fingernails, and toenails.

As used herein, the term “dermal location” refers to a region of a skin of a subject having any size and area. The dermal location may encompass a portion of skin of a subject such as, e.g., the scalp. The dermal location may include one or more layers of skin including, e.g., the epidermis and the dermis. In some cases, the dermal location includes a wound.

As used herein in its conventional sense, a “photosensitizer” or “photoreactive agent” or “photosensitizing agent” is a light-activated drug or compound. A photosensitizer may be defined as a substance that absorbs electromagnetic radiation, most commonly in the visible spectrum, and releases it as another form of energy, most commonly as reactive oxygen species and/or as thermal energy. In some cases, a photosensitizing agent is useful in photodynamic therapy. Such agents may be capable of absorbing electromagnetic radiation and emitting energy sufficient to exert a therapeutic effect, e.g., the impairment or destruction of unwanted cells or tissue, or sufficient to be detected in diagnostic applications. For example, the photosensitizer can be any chemical compound that collects in one or more types of selected target tissues and, when exposed to light of a particular wavelength, absorbs the light and induces impairment or destruction of the target tissues. Virtually any chemical compound that homes to a selected target and absorbs light may be used. The photosensitizer may be nontoxic to a subject to which it is administered and is capable of being formulated in a nontoxic composition, The photosensitizer may also be nontoxic in its photodegraded form. In some cases, the photosensitizers are characterized by a lack of toxicity to cells in the absence of the photochemical effect and are readily cleared from non-target tissues.

As used herein in its conventional sense, the term “wound” includes any disruption and/or loss of normal tissue continuity in an internal or external body surface of a human or non-human animal body, e.g. resulting from a non-physiological process such as surgery or physical injury. The expression “wound” or “wound environment” used herein refers to any skin lesion capable of triggering a healing process which may potentially lead to scarring, and includes wounds created by injury, wounds created by burning, wounds created by disease and wounds created by surgical procedures. The wound may be present on any external or internal body surface and may be penetrating or non-penetrating. The methods herein described may be beneficial in treating problematic wounds on the skin's surface. Examples of wounds which may be treated in accordance with the method of the invention include both superficial and non-superficial wounds, e.g. abrasions, lacerations, wounds arising from thermal injuries (e.g. burns and those arising from any cryo-based treatment), and any wound resulting from surgery.

The term “wound healing” as used herein in its conventional sense refers to a regenerative process with the induction of a temporal and spatial healing program, including, but not limited to, the processes of inflammation, granulation, neovascularization, migration of fibroblast, endothelial and epithelial cells, extracellular matrix deposition, re-epithelialization, and remodeling.

The term “hair follicle formation” or “induction of hair follicle formation” as used herein in its conventional sense refers to a phenomenon in which dermal papilla cells induce epidermal cells to form the structure of the hair follicle.

The term “hair growth” or “induction of hair growth” as used herein in its conventional sense refers to a phenomenon in which hair matrix cells of the hair follicle differentiate and proliferate thereby forming the hair shaft, and dermal sheath cells act on the hair matrix or outer root sheath (ORS) to elongate the hair shaft from the body surface. In some cases, hair growth includes generating one or more new hair follicles. In some cases, hair growth includes generating one or more new hairs.

As used herein in its conventional sense, the term “alopecia” refers to a disease in which hair is lost. It can be due to a number of causes, such as androgenetic alopecia, trauma, radiotherapy, chemotherapy, iron deficiency or other nutritional deficiencies, autoimmune diseases and fungal infection. The loss of hair in alopecia is not limited just to head hair but can happen anywhere on the body. Alopecia is often accompanied by fading of hair color. Alopecia is often accompanied by deterioration of hair quality such as hair becoming finer or hair becoming shorter. With regard to types of alopecia, there are alopecia areata, androgenetic alopecia, postmenopausal alopecia, female pattern alopecia, seborrheic alopecia, alopecia pityroides, senile alopecia, cancer chemotherapy drug-induced alopecia, alopecia due to radiation exposure, trichotillomania, postpartum alopecia, etc. The types of alopecia are further described in U.S. Pat. No. 9,808,511, the entirety of which is incorporated by reference herein.

Alopecia areata is an auto-immune disease that can cause hair to fall out suddenly. Alopecia areata is alopecia in which coin-sized circular to patchy bald area(s) with a clear outline suddenly occur, without any subjective symptoms or prodromal symptoms, etc. in many cases, and subsequently when spontaneous recovery does not occur they gradually increase in area and become intractable. It may lead to bald patches on the scalp or other parts of the body. Hair growth in the affected hair follicles is reduced or completely ceases. Alopecia areata is known to be associated with an autoimmune disease such as a thyroid disease represented by Hashimoto's disease, vitiligo, systemic lupus erythematosus, rheumatoid arthritis, or myasthenia gravis or an atopic disease such as bronchial asthma, atopic dermatitis, or allergic rhinitis.

As used herein in its conventional sense, the term “microneedling” refers to the use of microneedles on an area of the body. An individual microneedle is designed to puncture the skin up to a predetermined distance, which may be greater than the nominal thickness of the stratum corneum layer of skin (the very outer layer of the skin out-covering the epidermis). Using such microneedles may overcome the barrier properties of the skin. At the same time, the microneedles are relatively painless and bloodless if they are made to not penetrate through the epidermis, which is approximately less than 2.0-2.5 mm beneath the outer surface of the skin. Microneedles may require a direct pushing motion against the skin of sufficient force to penetrate completely through the stratum corneum. In general, microneedle stimulation systems are well known for their use in skin care treatment of various conditions such as wrinkles, acne scarring, stretch marks, skin whitening and facial rejuvenation. In certain embodiments of microneedling, a method of piercing holes in the skin and applying drugs or cosmetics to the skin provides a way to rapidly and sufficiently permeate the skin. In some cases, using microneedles is sufficient to injure the skin just enough to begin natural healing processes and stimulate collagen and elastin production, and the like, to heal the skin. In these methods, hundreds to thousands of tiny holes or rnicroconduits are created in the skin with the microneedling device without damaging the deeper layers of the skin. This injury to the skin begins a natural healing process that leads to the release of natural stimulants and growth factors which stimulates the formation of new natural collagen and elastin in the papillary dermis to produce new, healthy skin tissue. Also, new capillaries are formed. This neovascularisation and neocollagenesis associated with the wound healing process leads to the formation of younger looking skin, reduction of skin pathologies and improvement of scars. Generally called percutaneous collagen induction therapy, microneedling has also been used in the treatment of photo ageing. Furthermore, medical substances may be applied to the site where the holes are created and the substances are supposed to permeate into the skin through the tiny holes. Microneedling is generally applied to the face, neck, scalp, and just about anywhere on the body where a particular condition warrants without removing or permanently damaging the skin. A predetermined number of needles are inserted into the skin to the desired depth. As a reaction to the minor injury, the skin tissue begins a natural wound-healing cascade. This natural process forms new healthy dermal tissue that helps smooth scars, remove wrinkles and improve pigmentation, and yields a younger, healthier and a cleaner-looking skin.

As used herein in its conventional sense, the term “fractional laser resurfacing treatment” or “fractional laser resurfacing” or “fractional resurfacing” refers to the use of electromagnetic radiation to improve skin defects by inducing a thermal injury to the skin, which results in a complex wound healing response of the skin. This leads to a biological repair of the injured skin. Various techniques providing this objective have been introduced. The different techniques can be generally categorized in two groups of treatment modalities: ablative laser skin resurfacing (“LSR”) and non-ablative collagen remodeling (“NCR”). The first group of treatment modalities, i.e., LSR, includes causing thermal damage to the epidermis and/or dermis, while the second group, i.e., NCR, is designed to spare thermal damage of the epidermis. LSR with pulsed CO₂ or Er:YAG lasers, which may be referred to in the art as laser resurfacing or ablative resurfacing, is considered to be an effective treatment option for signs of photo aged skin, chronically aged skin, scars, superficial pigmented lesions, stretch marks, and superficial skin lesions. NCR techniques are variously referred to in the art as non-ablative resurfacing, non-ablative subsurfacing, or non-ablative skin remodeling. NCR techniques generally utilize non-ablative lasers, flashlamps, or radio frequency current to damage dermal tissue while sparing damage to the epidermal tissue. The concept behind NCR techniques is that the thermal damage of only the dermal tissues is thought to induce wound healing which results in a biological repair and a formation of new dermal collagen. This type of wound healing can result in a decrease of photoaging related structural damage. Avoiding epidermal damage in NCR techniques decreases the severity and duration of treatment related side effects. In particular, post procedural oozing, crusting, pigmentary changes and incidence of infections due to prolonged loss of the epidermal barrier function can usually be avoided by using the NCR techniques. Additional methods and devices for practicing fractional laser resurfacing are described in, e.g., PCT Application No. WO 2005/007003; U.S. Application No. 20160324578; and Beasley et al. (2013) Current Dermatology Reports. 2:135-143, the disclosures of which are incorporated herein by reference in their entireties.

As used herein, the term “administering” includes in vivo administration as well as direct administration to tissues ex vivo. Generally, administration is, for example, oral, buccal, parenteral (e.g., intravenous, intraarterial, subcutaneous), intraperitoneal (i.e., into the body cavity), topically, e.g., by inhalation or aeration (i.e., through the mouth or nose), or rectally systemic (i.e., affecting the entire body). A composition may be administered in dosage unit formulations containing conventional non-toxic pharmaceutically acceptable carriers, adjuvants, and vehicles as desired. The term “topically” may include injection, insertion, implantation, topical application, or parenteral application.

### DETAILED DESCRIPTION

Methods of promoting healing of a wound in a dermal location of a subject are provided. Aspects of the methods may include administering an effective amount of a YAP inhibitor composition to the wound to modulate mechanical activation of Engrailed-1 lineage-negative fibroblasts (ENFs) in the wound to promote ENF-mediated healing of the wound. Also provided are methods of preventing scarring during healing of a wound in a subject and methods of promoting hair growth on a subject. Aspects of the methods may include forming a wound in a dermal location of a subject and administering an effective amount of a YAP inhibitor composition to the wound to modulate mechanical activation of Engrailed-1 lineage-negative fibroblasts (ENFs) in the wound to promote ENF-mediated healing of the wound. Also provided are kits including an amount of a YAP inhibitor composition and a tissue disrupting device.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range, Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

In further describing various aspects of the invention, the methods are reviewed first in greater detail, followed by a review of kits. Applications in which the methods and kits find use are also provided in greater detail below.

### Methods

As summarized above, aspects of the methods include methods of promoting healing of a wound in a dermal location of a subject. In certain embodiments, the healing is ENF-mediated healing. In some cases, the methods prevent scarring during healing of a wound in a subject. In some cases, the methods promote hair growth on a subject. In certain embodiments, aspects of the methods include administering an effective amount of a YAP inhibitor composition to a wound to promote healing of the wound. In certain embodiments, aspects of the methods include administering an effective amount of a YAP inhibitor composition to the wound to modulate mechanical activation of Engrailed-1 lineage-negative fibroblasts (ENFs) in the wound to promote ENF-mediated healing of the wound. The methods may be applied to any cell or population of cells as described herein. The methods may include comparing an outcome with a control, e.g., a wound or healed wound not treated with a YAP inhibitor composition, dermal location including a scar, a dermal location lacking dermal appendages, or a dermal location lacking a scar.

In some cases, the methods include modulating mechanical signaling through a mechanical signaling pathway or mechano-transduction pathway in one or more cells , e.g., in a wound environment. The one or more cells may be any cell as described herein such as, e.g., ENFs. As used herein, the term “mechanical activation” refers to activation of a mechanical signaling pathway in one or more cells, e.g., one or more ENFs, that leads to, e.g., the expression and/or activity of Engrailed-1 (En-1) (Engrailed Homeobox 1) (Uniprot Accession No: Q05925) in the one or more cells in response to mechanical cues within a wound environment. The mechanical cues can include, e.g., mechanical tension, extracellular matrix (ECM) rigidity, strain, shear stress, or adhesive area. In some cases, activation of the mechanical signaling pathway in the one or more cells contributes to fibrosis and scarring after wounding. In some cases, the mechanical signaling pathway converts mechanical cues, e.g., in a wound environment, into transcriptional changes such as, e.g., expression of pro-fibrotic genes in the one or more cells. In some cases, the mechanical signaling pathway is activated when the one or more cells interact with their environment, e.g., probe the stiffness of their environment, through integrins and transmembrane receptors that couple to cell adhesion structures, e.g., focal adhesion kinase (FAK), to convert mechanical cues into transcriptional changes via Rho and Rho-associated protein kinase (ROCK) signaling. The mechanical signaling pathway may include Yes-Associated Protein (YAP; Yes-Associated Protein 1; YAP1) (Uniprot Accession No: P46937) as the final transcriptional effector, e.g., that activates pro-fibrotic genes. In some cases, the mechanical signaling pathway leads to transcriptional changes that include increasing the expression and/or activity of En-1 in the one or more cells in a wound environment. In some cases, the mechanical signaling pathway includes any one of the signaling pathways described, e.g., in Keely et al. (2011) Journal Of Cell Science 124:1195-1205.

In certain embodiments, the methods include modulating the mechanical activation of one or more cells, e.g., in a wound. The one or more cells may include ENFs. The mechanical activation of ENFs may promote a transition of ENFs, e.g., a subpopulation of ENFs, to

Engrailed-1 lineage-positive fibroblasts (EPFs), e.g., following wounding in the wound environment. The EPFs may be postnatally derived EPFs (pEPFs). In some cases, the methods may reduce or inhibit expression or activity of En-I ENFs such that the ENFs do not transition to EPFs. In some cases, the methods include reducing a transition of ENFs to EPFs in the wound, e.g., relative to a wound not treated with the YAP inhibitor composition. In some cases, the methods include inhibiting a transition of ENFs to EPFs in the wound. In some cases, the method includes preserving an amount of ENFs relative to an amount of EPFs present in the wound, e.g., a ratio of ENFs relative to EPFs. In these embodiments, one or more ENFs originally present in a wound environment following formation of the wound remain ENFs and do not, e.g., transition to EPFs via mechanical activation. In some cases, the method includes increasing the amount of ENFs relative to the amount of EPFs present in the wound compared to an amount of ENFs relative to an amount of EPFs present in a wound not treated with the YAP inhibitor composition (i.e., increasing the ratio of ENFs relative to EPFs present in the wound treated with the YAP inhibitor composition compared to the ratio of ENFs relative to EPFs present in a wound not treated with the YAP inhibitor composition), In some cases, the ratio of ENFs to EPFs in a wound ranges from 2:1 to 50:1, including, e.g., from 2:1 to 40:1, from 2:1 to 30:1, from 2:1 to 20:1, from 2:1 to 15:1, from 2:1 to 10:1, from 2:1 to 5:1. In some cases, the methods produce a wound or healed wound containing ENFs exclusively, wherein the wound or healed wound contains no EPFs or substantially no EPFs. The method may include quantitating the amount of ENFs and/or EPFs in the wound. The quantitating may occur by any convenient assay including, e.g., microscopy (e.g., fluorescence microscopy), flow cytometry, histological analysis, immunofluorescence, etc.

Cells of interest in the embodiments of the invention may include any cell present in the skin, In some cases, one or more cells of interest includes cells present in one or more layers of skin such as cells present in the dermis, i.e., dermal cells. In some cases, the one or more cells includes cells that participate in wound healing and/or scarring. In some cases, the one or more cells includes fibroblasts, e.g., dermal fibroblasts including, e.g., one or more subpopulations of dermal fibroblasts. In some cases, the one or more cells includes cells of a lineage derived from fibroblasts. In some cases, the one or more cells includes ENFs, e.g., dermal ENFs. ENFs of interest in the embodiments of the invention may include any number of sub-populations of ENFs, e.g., cells from one or more sub-populations of ENFs. In some cases, the ENFs include ENFs of the papillary dermis. In some cases, the ENFs include ENFs of the reticular dermis. In some cases, the ENFs include reticular dermal (Dlk1+) ENFs. In some cases, the ENFs include ENFs of the hypodermis.

As summarized above, aspects of the methods may include administering an effective amount of a YAP inhibitor composition to a wound. The administration may promote healing of a wound. In some cases, the administration modulates mechanical activation of one or more cells, e.g., ENFs, in the wound. In certain embodiments, the YAP inhibitor composition includes one or more YAP inhibitors. In some cases, the YAP inhibitor composition consists essentially of a YAP inhibitor. As used herein, a “YAP inhibitor” refers to a molecule that may inhibit YAP function and signaling. In some cases, the YAP inhibitor inhibits cellular mechanical signaling. In some cases, the YAP inhibitor reduces or inhibits YAP expression (DNA or RNA expression) or activity (e.g., nuclear translocation). In some cases, the YAP inhibitor reduces or inhibits the interaction of YAP with other signaling molecules, e.g., in a mechanical signaling pathway in one or more cells (e.g., ENFs) involved in fibrosis and scarring. In some cases, the YAP inhibitor reduces or inhibits transcriptional activation of targets downstream of YAP. In certain embodiments, administering the YAP inhibitor composition reduces mechanical activation of one or more cells, e.g., ENFs, in a wound, wherein, e.g., the level of mechanical activation of the one or more cells, e.g., ENFs, in a wound is reduced compared to the level of mechanical activation of one or more cells, e.g., ENFs, in a wound not treated with the YAP inhibitor composition. In some embodiments, administering the YAP inhibitor composition inhibits mechanical activation of one or more cells, e.g., ENFs, in a wound. In some cases, administering the YAP inhibitor composition reduces or inhibits the expression or activity of En-1 in one or more cells, e.g., ENFs. In some case, administering the YAP inhibitor composition reduces or inhibits a transition of ENFs to EPFs in the wound. In some cases, administering the YAP inhibitor composition preserves an amount of ENFs relative to an amount of EPFs present in the wound. In some cases, administering the YAP inhibitor composition increases the amount of ENFs relative to the amount of EPFs present in the wound compared to an amount of ENFs relative to an amount of EPFs present in a wound not treated with the YAP inhibitor composition.

As used herein, “an effective amount of a YAP inhibitor composition” refers to an amount of a YAP inhibitor composition suitable to promote healing of a wound and/or modulate the mechanical activation of one or more cells, e.g., ENFs, in a wound according to any of the embodiments of methods as described herein. In some cases, an effective amount of a YAP inhibitor composition includes one or more unit doses of the YAP inhibitor composition, such as, e.g., two or more doses, three or more doses, four or more doses, five or more doses, six or more doses, seven or more doses, eight or more doses, nine or more doses, or ten or more doses. In some cases, an effective amount of a YAP inhibitor composition includes a single dose, e.g., a single injection, of the YAP inhibitor composition. The YAP inhibitor composition may include any suitable amount of YAP inhibitor such as, e.g., an effective amount of a YAP inhibitor suitable to modulate the mechanical activation of one or more cells, e.g., ENFs, in a wound according to any of the embodiments of methods as described herein. In some cases, the effective amount of a YAP inhibitor composition does not delay wound closure or the wound closure rate. In some cases, the YAP inhibitor composition includes an effective amount of a YAP inhibitor ranging from, e.g., 0.1 mg/ml to 2 mg/ml, 0.5 mg/mI to 2 mg/ml, 1 mg/ml to 2 mg/ml, 0.1 mg/ml to 1 mg/ml, 0.5 mg/mI to 1 mg/ml, or 1 mg/ml to 5 mg/ml. The effective amount of the YAP inhibitor composition may be administered, e.g., after wound formation, over any suitable period of time including, e.g., one day or more, 2 days or more, 3 days or more, 4 days or more, 5 days or more, 7 days or more, 8 days or more, 9 days or more, 10 days or more, 11 days or more, 12 days or more, 13 days or more, 14 days or more, 21 days or more, 30 days or more, 60 days or more, or 90 days or more.

In some instances, the YAP inhibitor is a small molecule agent that exhibits the desired activity, e.g., inhibiting YAP expression and/or activity. Naturally occurring or synthetic small molecule compounds of interest include numerous chemical classes, such as organic molecules, e.g., small organic compounds having a molecular weight of more than 50 and less than about 2,500 Daltons. Candidate agents comprise functional groups for structural interaction with proteins, particularly hydrogen bonding, and typically include at least an amine, carbonyl, hydroxyl or carboxyl group, preferably at least two of the functional chemical groups. The candidate agents may include cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing the screening protocols.

In some cases, the YAP inhibitor is a photosensitizing agent. In some cases, the Yap inhibitor is a benzoporphyrin derivative (BPD). The benzoporphyrin derivative may be any convenient benzoporphyrin derivative such as, e.g., those described in U.S. Pat. Nos. 5,880,145; 6,878,253; 10,272,261; and U.S. Application No. 2009/0304803, the disclosures of which are incorporated herein by reference in their entireties. In some cases, the benzoporphyrin derivative is a photosensitizing agent. In some cases, the YAP inhibitor is verteporfin (benzoporphyrin derivative monoacid ring A, BPD-MA; tradename: Visudyne®).

In some cases, the YAP inhibitor is a protein or fragment thereof or a protein complex. In some cases, the YAP inhibitor is an antibody binding agent or derivative thereof. The term “antibody binding agent” as used herein includes polyclonal or monoclonal antibodies or fragments that are sufficient to bind to an analyte of interest, e.g., YAP. The antibody fragments can be, for example, monomeric Fab fragments, monomeric Fab′ fragments, or dimeric F(ab)′2 fragments. Also within the scope of the term “antibody binding agent” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies. In some cases, the YAP inhibitor is an enzyme or enzyme complex. In some cases, the YAP inhibitor includes a phosphorylating enzyme, e.g., a kinase. In some cases, the YAP inhibitor is a complex including a guide RNA and a CRISPR effector protein, e.g., Cas9, used for targeted cleavage of a nucleic acid.

In some cases, the YAP inhibitor is a nucleic acid. The nucleic acids may include DNA or RNA molecules. In certain embodiments, the nucleic acids modulate, e.g., inhibit or reduce, the activity of a gene or protein, e.g., by reducing or downregulating the expression of the gene. The nucleic acid may be a single stranded or double-stranded and may include modified or unmodified nucleotides or non-nucleotides or various mixtures and combinations thereof. In some cases, the YAP inhibitor includes intracellular gene silencing molecules by way of RNA splicing and molecules that provide an antisense oligonucleotide effect or an RNA interference (RNAi) effect useful for inhibiting gene function. In some cases, gene silencing molecules, such as, e.g., antisense RNA, short temporary RNA (stRNA), double-stranded RNA (dsRNA), small interfering RNA (siRNA), short hairpin RNA (snRNA), microRNA (miRNA), tiny non-coding RNA (tncRNA), snRNA, snoRNA, and other RNAi-like small RNA constructs, may be used to target a protein-coding as well as non-protein-coding genes. In some case, the nucleic acids include aptamers (e.g., spiegelmers). In some cases, the nucleic acids include antisense compounds. In some cases, the nucleic acids include molecules which may be utilized in RNA interference (RNAi) such as double stranded RNA including small interfering RNA (siRNA), locked nucleic acid (LNA) inhibitors, peptide nucleic acid (PNA) inhibitors, etc.

In some embodiments, the YAP inhibitor composition is administered as a pharmaceutically acceptable composition in which one or more YAP inhibitors may be mixed with one or more carriers, thickeners, diluents, buffers, preservatives, surface active agents, excipients and the like. Pharmaceutical compositions may also include one or more additional active ingredients such as antimicrobial agents, anti-inflammatory agents, anesthetics, and the like in addition to the one or more YAP inhibitors. In some cases, the YAP inhibitor composition includes, e.g., a derivative of YAP inhibitor. “Derivatives” include pharmaceutically acceptable salts and chemically modified agents.

The pharmaceutical compositions of the present invention may be administered by, any route commonly used to administer pharmaceutical compositions. For example, administration may be done topically (including opthaimically, vaginaily, rectally, intranasally), orally, by inhalation, or parenterally, for example by intravenous drip or subcutaneous, intraperitoneal or intramuscular injection.

Pharmaceutical compositions formulated for topical administration may include ointments, lotions, creams, gels, drops, sprays, liquids, salves, sticks, soaps, aerosols, and powders. Any conventional pharmaceutical excipient, such as carriers, aqueous, powder or oily bases, thickeners and the like may be used. Ointments and creams may, for example, be formulated with an aqueous or oily base with the addition of suitable thickening and/or gelling agents. Lotions may be formulated with an aqueous or oily base and will, in general, also contain one or more emulsifying, dispersing, suspending, thickening or coloring agents. Powders may be formed with the aid of any suitable powder base. Drops may be formulated with an aqueous or non-aqueous base also comprising one or more dispersing, solubilising or suspending agents. Aerosol sprays are conveniently delivered from pressurised packs, with the use of a suitable propellant.

The YAP inhibitor composition may be stored at any suitable temperature. In some cases, the YAP inhibitor composition is stored at temperatures ranging from 1° C. to 30° C., from 2° C. to 27° C., or from 5° C. to 25° C. The YAP inhibitor composition may be stored in any suitable container, as described in detail below.

The YAP inhibitor composition may be administered to a wound in a dermal location a subject. In some cases, the YAP inhibitor composition is administered to a dermal location surrounding a wound in a subject. The administration can be by any suitable route, including, e.g., topical, intravenous, subcutaneous, and intramuscular. In some cases, the administering comprises injecting the composition below a topical dermal location of the subject. The injecting may be performed with any suitable device such as, e.g., a needle. Other delivery means include coated microneedles, i.e. microneedles having a YAP inhibitor composition deposited thereon, as well as microneedles that include internal reservoirs that are configured to receive a YAP inhibitor composition therein and disperse the YAP inhibitor composition therefrom. In some cases, the administering comprises delivering the composition to a topical dermal location. The delivering may be performed with any suitable device or composition such as, e.g., a transdermal patches, gels, creams, ointments, sprays, lotions, salves, sticks, soaps, powders, pessaries, aerosols, drops, solutions and any other convenient pharmaceutical forms. The YAP inhibitor composition may be administered at any suitable time. In some cases, the YAP inhibitor composition is administered to a wound immediately after formation of the wound in a subject. In some cases, the YAP inhibitor composition is administered to a wound after any suitable amount of time after formation of the wound such as, e.g., 1 minute, 2 minutes, 5 minutes, 10 minutes, 30 minutes, or an hour after formation of the wound. In certain embodiments, the methods as provided herein promote healing of a wound. In certain embodiments, the methods as provided herein promote ENF-mediated healing of a wound. As used herein, the term “ENF-mediated healing” refers to healing of a wound associated with the presence and/or activity of ENFs in the wound. In some cases, the healing, e.g., ENF-mediated healing, includes a regenerative response from one or more cells . In some cases, the methods do not compromise healing of a wound, e.g., wound closure and repair. For example, in some cases, the methods do not delay wound closure or the wound closure rate. In some cases, the healing, e.g., ENF-mediated healing, of the wound is completed in an amount of time substantially equal to an amount of time for healing of a wound not treated with the YAP inhibitor composition. In some cases, the healing, e.g., ENF-mediated healing, of the wound is completed in an amount of time that is less than an amount of time for healing of a wound not treated with the YAP inhibitor composition, i.e., the healing, e.g., ENF-mediated healing, of the wound is accelerated compared to the healing of a wound not treated with the YAP inhibitor composition. In certain embodiments, the methods reduce or prevent scarring during healing of a wound in a subject, as described in detail below. In some cases, the healing, e.g., ENF-mediated healing, of the wound includes regeneration of dermal appendages. In some cases, the dermal appendages include hair follicles, sweat glands, and sebaceous glands. In certain embodiments, the methods provided herein promote hair growth on a subject, as described in detail below. In certain embodiments, the methods provided herein treat a subject for alopecia, e.g., by promoting hair growth in areas of hair loss, as described in detail below. In some cases, the healing, e.g., ENF-mediated healing, of the wound produces a healed wound with reduced levels of collagen hyperproliferation compared to levels of collagen hyperproliferation in a healed wound not treated with the YAP inhibitor composition. In some cases, the healing, e.g., ENF-mediated healing, of the wound produces a healed wound comprising improved connective tissue architecture compared to the connective tissue architecture in a healed wound not treated with the YAP inhibitor composition. In certain embodiments, the healing, e.g., ENF-mediated healing, includes recovery or regrowth of one or more of dermal appendages, ultrastructure (i.e., matrix structure), and mechanical strength (e.g., wound breaking strength) that is, e.g., comparable to that of normal skin or unwounded skin.

In certain embodiments, the methods further include forming a wound in a dermal location of a subject. In some cases, the wound is formed to perform a procedure, e.g., a surgical procedure, In some cases, the wound is formed to improve tissue quality, For example, the methods may include forming microscopic injuries to induce tissue regeneration. In some cases, the wound is formed to disrupt an outer dermal layer, e.g., stratum corneum, to increase penetration and absorption of one or more substances or compositions, e.g., a therapeutic composition, through the skin of a subject. In some cases, the methods include forming one or more wounds at a plurality of dermal locations. In some cases, the methods include forming one or more wounds across a dermal location. The nature and size of the wound may vary. In certain embodiments, the wound is a microscopic wound. The microscopic wound may be formed by any suitable means as described in detail below such as, e,g., a laser, microneedle, etc. In certain embodiments, the wound is a partially healed wound.

The wound may be formed by any suitable means, e.g., mechanical, physical or chemical injury of the skin, In some cases, the wound results from non-physiological processes, e.g., a surgical wound or a wound resulting from physical injury, abrasions, lacerations, thermal injuries (e.g,, a burn or a wound arising from a cryo-based treatment). In some cases, the wound is formed by the application of one or more of, e.g., ultrasound, radio frequency (RF), laser (e.g., fraxel), ultraviolet energy, infrared energy, or mechanical disruption. In some cases, the wound is formed by, e.g., microdermabrasion (e.g., with an adapted skin preparation pad, sandpaper), microneedling, tape-stripping, pan-scrubber, exfoliating scrub, compress rubbing, non ablative lasers at a low-energy delivery. Additional mechanical treatments include, e.g., curettage or dermoabrasion (e.g., with an adapted sandpaper or micro-needling (or micro-perforation)). In certain aspects, wounding is accomplished using chemical treatments (e.g., a caustic agent, etc), or mechanical or electromagnetic or physical treatments including but not limited to dermabrasion (DA), particle-mediated dermabrasion (PMDA), microdermabrasion, microneedles, laser (e.g., a laser that delivers ablative, non-ablative, fractional, non-fractional, superficial, or deep treatment, and/or that is CO₂-based, or erbium-YAG-based, erbium-glass based (e.g. Sciton Laser), neodyrniurn:yttriurn aluminum garnet (Nd:YAG) laser, etc.), a low-level (low-intensity) laser therapy treatment (e.g., HairMax® Laser comb), laser abrasion, irradiation, radio frequency (RF) ablation, dermatome planing (e.g. dermaplaning), a coring needle, a puncture device, a punch tool or other surgical tool, suction tool or instrument, electrology, electromagnetic disruption, electroporation, sonoporation, low voltage electric current, intense pulsed light, or surgical treatments (e.g., skin graft, hair transplantation, strip harvesting, scalp reduction, hair transplant, follicular unit extraction (FUE), robotic FUE, etc.), or supersonically accelerated saline. In some cases, the wound is formed by a tissue disrupting device, as described in detail below.

Embodiments of the methods of the present invention can be practiced on any suitable subject. A subject of the present invention may be a “mammal” or “mammalian”, where these terms are used broadly to describe organisms which are within the class mammalia, including the orders carnivore (e,g., dogs and cats), rodentia (e.g., mice, guinea pigs, and rats), and primates (e.g., humans, chimpanzees, and monkeys). In some instances, the subjects are humans. The methods may be applied to human subjects of both genders and at any stage of development (i.e., neonates, infant, juvenile, adolescent, adult), where in certain embodiments the human subject is a juvenile, adolescent or adult. While the present invention may be applied to samples from a human subject, it is to be understood that the methods may also be carried-out on samples from other animal subjects (that is, in “non-human subjects”) such as, but not limited to, birds, mice, rats, dogs, cats, livestock and horses.

### Scar Reduction

In certain embodiments, the methods provided herein reduce or prevent scarring during healing of a wound in a subject. In certain embodiments, the methods include forming a wound in a dermal location of a subject, e.g., according to any of the embodiments described herein, and administering an effective amount of a YAP inhibitor composition to the wound to promote healing of the wound, e.g., according to any of the embodiments described herein. In certain embodiments, the methods include forming a wound in a dermal location of a subject, e.g., according to any of the embodiments described herein, and administering an effective amount of a YAP inhibitor composition to the wound to modulate mechanical activation of Engrailed-1 lineage-negative fibroblasts (ENFs) in the wound to promote ENF-mediated healing of the wound, e.g., according to any of the embodiments described herein. In some cases, the administration of a YAP inhibitor composition according to any of the embodiments described herein reduces or prevents scarring by targeting the expression and/or activity of YAP in ENFs, e.g., Dlk+ reticular ENFs.

The level or amount of scarring may be assessed and measured according to any convenient metric. The levels of scarring, e.g., in a wound treated with a YAP inhibitor composition during healing or a healed wound treated with a YAP inhibitor composition, may be assessed relative to a control, e.g., a wound or healed wound not treated with a YAP inhibitor composition, In some cases, the level of scarring is assessed by measuring a physical property of a healed wound such as, e.g., tensile strength, scar area, etc. In some cases, the level of scarring is assessed by detecting the presence of or quantitating the amount of one or more dermal appendages including, e.g., hair follicles, sweat glands, and sebaceous glands, in a dermal location. In some cases, the level of scarring is assessed by detecting and/or characterizing the formation of connective tissue or an ECM matrix in a dermal location. In certain embodiments, the level of scarring is assessed by detecting and/or quantitating the amount of cells, e.g., types or subpopulations of cells, in a dermal location. In some cases, the level of scarring is assessed by detecting and/or quantitating the amount of one or more of ENFs and EPFs. In certain embodiments, the level of scarring is assessed by quantitating the amount of ENFs relative to the amount of EPFs in a dermal location. In some cases, the level of scarring is assessed by measuring and/or quantitating the expression and/or activity or one or more scar-related genes and/or scar-related gene products. In some cases, levels of scarring are assessed by one or more of the following: visual examination, histology, immunohistochemical analysis, immunofluorescence, and machine learning. In some cases, the level of scarring is assessed with a machine learning algorithm for quantitatively assessing connective tissue and fibrosis based on histological stains. In some embodiments, evaluated metrics include, e,g., ECM fiber length and width, packing and alignment of groups of ECM fibers, and ECM fiber branching. Various scar assessment scales are provided, e.g., in POT Application No. WO 2014/040074, the disclosure of which is incorporated herein by reference in its entirety. According to some embodiments, the methods reduce scarring compared to a control as measured by visual analog scale (VAS) score, color matching (CM), matte/shiny (M/S) assessment, contour (C) assessment, distortion (D) assessment, texture (T) assessment, or a combination thereof. While the magnitude of scarring reduction may vary, in some instances the magnitude ranges from 10% to 98%, such as, 10% to 95%, 20% to 95%, 30% to 95%, 40% to 95%, 50% to 95%, 60% to 95%, 70% to 95%, 80% to 95%, or 90% to 95%,

The levels of reduction of scarring during the healing process may vary. In certain embodiments, the methods are effective to reduce the occurrence, severity, or both of scars. In some cases, the method produces a healed wound with reduced levels of scarring compared to levels of scarring in a healed wound not treated with the YAP inhibitor composition. In certain embodiments, the method produces a scar-less healed wound. In some cases, the methods produce a healed wound comprising improved connective tissue architecture compared to the connective tissue architecture in a healed wound not treated with the YAP inhibitor composition. In some cases, the methods produce a healed wound with reduced levels of collagen hyperproliferation compared to levels of collagen hyperproliferation in a healed wound not treated with the YAP inhibitor composition. In some embodiments, the methods improve the alignment of collagen fibers in the wound. In some embodiments, the methods reduce collagen formation in the wound. In some cases, the methods produce a healed wound with increased growth of dermal appendages. In certain embodiments, the methods reduce the wound size. In some case, a dermal location having a healed wound treated with a YAP inhibitor composition according to the methods provided herein is indistinguishable in appearance (e.g., pigmentation, texture) from normal skin or unwounded skin. In some case, a dermal location having a healed wound treated with a YAP inhibitor composition according to the methods provided herein has physical properties (e.g., tensile strength) indistinguishable from normal skin or unwounded skin. In some cases, a dermal location having a healed wound treated with a YAP inhibitor composition according to the methods provided herein has growth and generation of dermal appendages that are indistinguishable from normal skin or unwounded skin. In some cases, a dermal location having a healed wound treated with a YAP inhibitor composition according to the methods provided herein has a connective tissue architecture, e.g., ECM matrix, that is indistinguishable from normal skin or unwounded skin. In certain embodiments, the methods do not impair normal wound healing or delay the wound closure rate compared to a control. In certain embodiments, the methods increase wound healing, e.g., the wound closure rate compared to a control. In some cases, one or more of the produced effects of the methods as described herein indicate a reduction of scarring or the prevention of scarring.

According to some embodiments, the methods decrease scar area compared to a control. According to some embodiments, the methods decrease scar area compared to a control by 1% or more, 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more. 45% or more, 50% or more, 55% or more, 60% or more, 65% or more, 70% or more, 75% or more, 80% or more, 85% or more, 90% or more, or 95% or more. According to some embodiments, the methods decrease scar area compared to a control within one day or more, 2 days or more, 3 days or more, 4 days or more, 5 days or more, 7 days or more, 8 days or more. 9 days or more, 10 days or more, 11 days or more, 12 days or more, 13 days or more, 14 days or more, 21 days or more, 30 days or more, 60 days or more, or 90 days or more of the administration, e.g., of a YAP inhibitor composition.

According to some embodiments, the methods decrease fibrosis at a dermal location compared to a contra In some cases, the methods decrease fibrosis at a dermal location compared to a control by 1% or more, 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more. According to some embodiments, the methods decrease fibrosis at a dermal location compared to a control within 1 day or more, 2 days or more, 3 days or more, 4 days or more, 5 days or more, 7 days or more, 8 days or more, 9 days or more, 10 days or more, 11 days or more, 12 days or more, 13 days or more, 14 days or more, 21 days or more, 30 days or more, 60 days or more, or 90 days or more of the administration.

According to some embodiments, the methods produce a wound or healed wound with increased tensile strength, e.g., as measured by wound breaking force and Young's modulus, compared to a control. According to some embodiments, the methods increase tensile strength compared to a control within one day or more, 2 days or more, 3 days or more, 4 days or more, 5 days or more, 7 days or more, 8 days or more, 9 days or more, 10 days or more, 11 days or more, 12 days or more, 13 days or more, 14 days or more, 21 days or more, 30 days or more, 60 days or more, or 90 days or more of the administration. According to some embodiments, the methods increase tensile strength compared to a control by 1% or more, 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more.

According to some embodiments, the methods produce detectible levels of dermal appendages such as hair follicles, sweat glands, and/or sebaceous glands, or any combination thereof, at a dermal location compared to a control. According to some embodiments, the methods increase the number of dermal appendages such as hair follicles, sweat glands, and/or sebaceous glands, or any combination thereof, at a dermal location compared to a control. In some cases, the methods increase the number of dermal appendages such as hair follicles, sweat glands, and/or sebaceous glands, or any combination thereof, at a dermal location compared to a control by 1% or more, 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more. According to some embodiments, the methods produce detectible levels of or increase the number of dermal appendages such as hair follicles, sweat glands, and/or sebaceous glands, or any combination thereof, at a dermal location compared to a control within 1 day or more, 2 days or more, 3 days or more, 4 days or more, 5 days or more, 7 days or more, 8 days or more, 9 days or more, 10 days or more, 11 days or more, 12 days or more, 13 days or more, 14 days or more, 21 days or more, 30 days or more, 60 days or more, or 90 days or more of the administration.

According to some embodiments, the methods increase the number of hairs at a dermal location compared to a control. In some cases, the methods increase the number of hairs at a dermal location compared to a control by 1% or more, 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more. According to some embodiments, the methods increase the number of hairs at a dermal location compared to a control within 1 day or more, 2 days or more, 3 days or more, 4 days or more, 5 days or more, 7 days or more, 8 days or more, 9 days or more, 10 days or more, 11 days or more, 12 days or more, 13 days or more, 14 days or more, 21 days or more, 30 days or more, 60 days or more, or 90 days or more of the administration.

In some cases, the methods modulate the amount and/or type of cells present in a wound. In some cases, the methods modulate the amount and/or type of one or more subpopulations of cells present in a wound. In some cases, the methods modulate the amount of ENFs or the amount of ENFs relative to the amount of EPFs in a wound or healed wound compared to a control. In some cases, the methods modulate the amount of DLK+ cells present in a wound or healed wound compared to a control, In some cases, the methods modulate the amount of YAP+ cells in a wound or healed wound compared to a control. In some cases, an increased amount of ENFs relative to the amount of EPFs present in the wound compared to an amount of ENFs relative to an amount of EPFs present in a control indicates a reduction in scarring or the prevention of scarring. In some cases, a reduction in the transition of ENFs to EPFs in the wound relative to a control indicates a reduction in scarring or the prevention of scarring. In some cases, the inhibition of the transition of ENFs to EPFs in the wound indicates a reduction in scarring or the prevention of scarring. In some cases, the preservation of an amount of ENFs relative to an amount of EPFs present in the wound, e.g., a ratio of ENFs relative to EPFs, indicates a reduction in scarring or the prevention of scarring. In some cases, a wound or healed wound containing ENFs exclusively indicates a reduction in scarring or the prevention of scarring. In some cases, a wound or healed wound containing a decreased amount of EPFs relative to a control indicates a reduction in scarring or the prevention of scarring.

According to some embodiments, the methods increase the amount of ENFs or the amount of ENFs relative to the amount of EPFs compared to a control. In certain embodiments, the methods increase the amount of ENFs or the amount of ENFs relative to the amount of EPFs by 1% or more, 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more. According to some embodiments, the methods increase the amount of ENFs or the amount of ENFs relative to the amount of EPFs compared to a control within one day or more, 2 days or more, 3 days or more, 4 days or more, 5 days or more, 7 days or more, 8 days or more, 9 days or more, 10 days or more, 11 days or more, 12 days or more, 13 days or more, 14 days or more, 21 days or more, 30 days or more, 60 days or more, or 90 days or more of the administration, e.g., of a YAP inhibitor composition.

According to some embodiments, the methods increase the amount of DLK+ cells present in a wound or healed wound compared to a control. In certain embodiments, the methods increase the amount of DLK+ cells present in a wound or healed wound by 1% or more, 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more. According to some embodiments, the methods increase the amount of DLK+ cells present in a wound or healed wound compared to a control within one day or more, 2 days or more, 3 days or more, 4 days or more, 5 days or more, 7 days or more, 8 days or more, 9 days or more, 10 days or more, 11 days or more, 12 days or more, 13 days or more, 14 days or more, 21 days or more, 30 days or more, 60 days or more, or 90 days or more of the administration, e.g., of a YAP inhibitor composition.

According to some embodiments, the methods decrease the amount of YAP+ cells, e.g., in a wound or a healed wound, compared to a control. In certain embodiments, the methods decrease the amount of YAP+ cells by 1% or more, 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more. According to some embodiments, the methods decrease the amount of YAP+ cells compared to a control within one day or more, 2 days or more, 3 days or more, 4 days or more, 5 days or more, 7 days or more, 8 days or more, 9 days or more, 10 days or more, 11 days or more, 12 days or more, 13 days or more, 14 days or more, 21 days or more, 30 days or more, 60 days or more, or 90 days or more of the administration, e.g., of a YAP inhibitor composition.

In some embodiments, the methods may modulate the expression and/or activity of scar-related genes or the production of scar-related gene products. In some cases, the level of scarring may be assessed by measuring the expression and/or activity of scar-related genes. In some cases, the level of scarring may be assessed by measuring the amount and/or activity of scar-related gene products. According to another embodiment, an effective amount of a YAP inhibitor composition is effective to modulate messenger RNA (mRNA) levels expressed from scar-related genes. According to another embodiment, an effective amount of a YAP inhibitor composition is effective to modulate the level of scar-related gene product expressed from the scar related gene. According to some embodiments, the scar-related gene and/or product is transforming growth factor-β1 (TGF-β1), tumor necrosis factor-α (TNF-α), collagen, interleukin-6 (IL-6), chemokine (CC motif) Ligand 2 (CCL2) (or monocyte chemotactic protein-1 (MCP-1)), chemokine (CC motif) receptor 2 (CCR2), EGF-like module-containing mucin-like hormone receptor-like 1 (EMR1), CD26, YAP, fibronectin, or one or more of the sma/mad-related proteins (SMAD). According to some embodiments, the methods modulate, e.g., decrease, the expression and/activity of one or more of collagen type 1, CD26, and YAP in a wound, e.g., in cells present in a wound, compared to a control. According to some embodiments, the methods modulate, e.g., increase, the expression and/activity of fibronectin in a wound, e.g., in cells present in a wound, compared to a control. According to some embodiments, the methods produce detectible levels of markers of hair follicle and sweat gland identity such as, e.g., cytokeratin 14 and/or cytokeratin 19, respectively, at a dermal location compared to a control. In some cases, the methods increase the levels of markers of hair follicle and sweat gland identity, e.g., cytokeratin 14 and/or cytokeratin 19, at a dermal location compared to a control.

In certain embodiments, the methods decrease or increase the expression and/activity of one or more scar-related genes or scar-related gene products by 1% or more, 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more. According to some embodiments, the methods decrease or increase the expression and/or activity of one or more scar-related genes or scar-related gene products compared to a control within one day or more, 2 days or more, 3 days or more, 4 days or more, 5 days or more, 7 days or more, 8 days or more, 9 days or more, 10 days or more, 11 days or more, 12 days or more, 13 days or more, 14 days or more, 21 days or more, 30 days or more, 60 days or more, or 90 days or more of the administration, e.g., of a YAP inhibitor composition.

### Hair Growth

In certain embodiments, the methods provided herein promote hair growth on a subject in a dermal location. In some embodiments, the subject may have alopecia and/or have been diagnosed with alopecia. In certain embodiments, the methods are methods for treating a subject for alopecia, e.g., by promoting hair growth in a dermal location of hair loss. In certain embodiments, the methods include forming a wound in a dermal location of a subject where hair growth is desired, e.g., according to any of the embodiments described herein, and administering an effective amount of a YAP inhibitor composition to the wound to promote healing of the wound, e.g., according to any of the embodiments described herein. In certain embodiments, the methods may include forming a wound in a dermal location where hair growth is desired of a subject, e.g., according to any of the embodiments described herein, and administering an effective amount of a YAP inhibitor composition to the wound to modulate mechanical activation of Engrailed-1 lineage-negative fibroblasts (ENFs) in the wound to promote ENF-mediated healing of the wound, e.g., according to any of the embodiments described herein. In some cases, the administration of a YAP inhibitor composition according to any of the embodiments described herein promotes hair growth by targeting the expression and/or activity of YAP in ENFs, e.g., Dlk+ reticular ENFs.

In certain embodiments, the methods provided herein promote hair growth on a subject. The methods may induce or promote hair growth at any suitable dermal location in a subject. In certain embodiments, the methods promote or induce hair growth in a dermal location devoid of dermal appendages, e.g., hair follicles, sweat glands, etc. In some cases, the dermal location is hairless. In some cases, the dermal location includes a scar. In certain embodiments, the methods promote or induce hair growth in a dermal location having dermal appendages, In some cases, the dermal location includes hair. The dermal location may be located at any portion of the body where hair may naturally grow such as, e.g., the scalp, face, legs, arms, etc.

In certain embodiments, the dermal location is present on a hairless area of the scalp of a subject, In certain embodiments, the dermal location includes the entire surface of the scalp of a subject.

The level of hair growth may be assessed and measured according to any convenient metric, The levels of hair growth may be assessed relative to a control, e.g., a dermal location characterized by hair loss, a dermal location devoid of dermal appendages, a wound not treated with a YAP inhibitor composition, or healed wound not treated with a YAP inhibitor composition. In certain embodiments, hair growth is determined by detecting the presence of new hairs appearing in a dermal location. In this method, hair growth may be confirmed when tips of the new hairs appear on the treatment area. Hair growth may also be determined by detecting hair follicle formation and/or measuring an increase in length of the hair follicles. In some cases, hair growth includes generating one or more new hair follicles. Hair growth may also be determined by measuring a change in the hairline. In some cases, the change in the hairline is determined by measuring the change in distance between any point on the hairline and the browline of the subject's head. In some cases, the methods decrease the amount of hair falling out compared to a control. In some cases, the methods prevent the progress of hair loss. In certain embodiments, there is no recurrence of hair loss permanently or for a period of time after performing the methods including, e.g., one month or more, two months or more, three months or more, half a year or more, one year or more, two years or more, three year or more, five years or more, or ten years or more.

According to some embodiments, the methods decrease the amount of hair loss compared to a control. In some cases, the methods decrease the amount of hair loss compared to a control by 1% or more, 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more. According to some embodiments, the methods decrease the amount of hair loss compared to a control within 1 day or more, 2 days or more, 3 days or more, 4 days or more, 5 days or more, 7 days or more, 8 days or more, 9 days or more, 10 days or more, 11 days or more, 12 days or more, 13 days or more, 14 days or more, 21 days or more, 30 days or more, 60 days or more, or 90 days or more of the administration, e.g., of a YAP inhibitor composition.

According to some embodiments, the methods increase the number of hair follicles at a dermal location, e.g., treated with a YAP inhibitor composition, compared to a control. In some cases, the methods increase the number of hair follicles at a dermal location compared to a control by 1% or more, 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more, According to some embodiments, the methods increase the number of hair follicles at a dermal location compared to a control within 1 day or more, 2 days or more, 3 days or more, 4 days or more, 5 days or more, 7 days or more, 8 days or more, 9 days or more, 10 days or more, 11 days or more, 12 days or more, 13 days or more, 14 days or more, 21 days or more, 30 days or more, 60 days or more, or 90 days or more of the administration, e.g., of a YAP inhibitor composition.

According to some embodiments, the methods increase the number of hairs at a dermal location compared to a control. In some cases, the methods increase the number of hairs at a dermal location compared to a control by 1% or more, 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more. According to some embodiments, the methods increase the number of hairs at a dermal location compared to a control within 1 day or more, 2 days or more, 3 days or more, 4 days or more, 5 days or more, 7 days or more, 8 days or more, 9 days or more, 10 days or more, 11 days or more, 12 days or more, 13 days or more, 14 days or more, 21 days or more, 30 days or more, 60 days or more, or 90 days or more of the administration, e.g., of a YAP inhibitor composition.

### Kits

Aspects of the present disclosure also include kits. The kits are suitable for practicing embodiments of the methods described herein. The kits may include, e.g., an amount of a YAP inhibitor composition and a tissue disrupting device. In some cases, the kits are suitable for practicing embodiments of the methods for promoting hair growth. In some cases, the kits are suitable for practicing embodiments of the methods for treating a subject for alopecia.

The YAP inhibitor composition may be present in any suitable amount. In some cases, the kit includes an effective amount of a YAP inhibitor composition, e.g., according to the embodiments described above. The YAP inhibitor composition may be present in any suitable container that is compatible with the YAP inhibitor composition. By “compatible” is meant that the container is substantially inert (e.g., does not significantly react with) the liquid and/or reagent(s) of the YAP inhibitor composition in contact with a surface of the container. Containers of interest may vary and may include but are not limited to a test tube, centrifuge tube, culture tube, falcon tube, microtube, Eppendorf tube, specimen collection container, specimen transport container, and syringe.

The container for holding the YAP inhibitor composition may be configured to hold any suitable volume of the YAP inhibitor composition. In some cases, the size of the container may depend on the volume of YAP inhibitor composition to be held in the container. In certain embodiments, the container may be configured to hold an amount of YAP inhibitor composition ranging from 0.1 mg to 1000 mg, such as from 0.1 mg to 900 mg, such as from 0.1 mg to 800 mg, such as from 0.1 mg to 700 mg, such as from 0.1 mg to 600 mg, such as from 0,1 mg to 500 mg, such as from 0.1 mg to 400 mg, or 0.1 mg to 300 mg, or 0.1 mg to 200 mg, or 0.1 mg to 100 mg, 0.1 mg to 90 mg, or 0.1 mg to 80 mg, or 0.1 mg to 70 mg, or 0.1 mg to 60 mg, or 0.1 mg to 50 mg, or 0.1 mg to 40 mg, or 0.1 mg to 30 mg, or 0.1 mg to 25 mg, or 0.1 mg to 20 mg, or 0.1 mg to 15 mg, or 0.1 mg to 10 mg, or 0.1 mg to 5 mg, or 0.1 mg to 1 mg, or 0.1 mg to 0.5 mg. In certain embodiments, the container is configured to hold an amount of YAP inhibitor composition ranging from 0.1 g to 10 g, or 0.1 g to 5 g, or 0.1 g to 1 g, or 0.1 g to 0.5 g. In certain instances, the container is configured to hold a volume (e.g., a volume of a liquid YAP inhibitor composition) ranging from 0.1 ml to 200 ml. For instance, the container may be configured to hold a volume (e.g., a volume of a liquid) ranging from 0.1 ml to 1000 ml, such as from 0.1 ml to 900 ml, or 0.1 ml to 800 ml, or 0.1 ml to 700 ml, or 0.1 ml to 600 ml, or 0.1 ml to 500 ml, or 0.1 ml to 400 ml, or 0.1 ml to 300 ml, or 0.1 ml to 200 ml, or 0.1 ml to 100 ml, or 0.1 ml to 50 ml, or 0.1 ml to 25 ml, or 0.1 ml to 10 ml, or 0.1 ml to 5 ml, or 0.1 ml to 1 ml, or 0.1 ml to 0.5 ml. In certain instances, the container is configured to hold a volume (e.g., a volume of a liquid YAP inhibitor composition) ranging from 0.1 ml to 200 ml.

The shape of the container may also vary. In certain cases, the container may be configured in a shape that is compatible with the assay and/or the method or other devices used to perform the assay. For instance, the container may be configured in a shape of typical laboratory equipment used to perform the assay or in a shape that is compatible with other devices used to perform the assay. In some embodiments, the liquid container may be a vial or a test tube. In certain cases, the liquid container is a vial, In certain cases, the liquid container is a test tube.

As described above, embodiments of the container can be compatible with the YAP inhibitor composition in contact with the reagent device. Examples of suitable materials for the containers include, but are not limited to, glass and plastic. For example, the container may be composed of glass, such as, but not limited to, silicate glass, borosilicate glass, sodium borosilicate glass (e.g., PYREX™), fused quartz glass, fused silica glass, and the like. Other examples of suitable materials for the containers include plastics, such as, but not limited to, polypropylene, polymethylpentene, polytetrafluoroethylene (PTFE), perfluoroethers (PFE), fluorinated ethylene propylene (FEP), perfluoroalkoxy alkanes (PFA), polyethylene terephthalate (PET), polyethylene (PE), polyetheretherketone (PEEK), and the like.

In some embodiments, the container may be sealed. That is, the container may include a seal that substantially prevents the contents of the container from exiting the container. The seal of the container may also substantially prevent other substances from entering the container. For example, the seal may be a water-tight seal that substantially prevents liquids from entering or exiting the container, or may be an air-tight seal that substantially prevents gases from entering or exiting the container. In some instances, the seal is a removable or breakable seal, such that the contents of the container may be exposed to the surrounding environment when so desired, e.g., if it is desired to remove a portion of the contents of the container. In some instances, the seal is made of a resilient material to provide a barrier (e.g., a water-tight and/or air-tight seal) for retaining a sample in the container. Particular types of seals include, but are not limited to, films, such as polymer films, caps, etc., depending on the type of container. Suitable materials for the seal include, for example, rubber or polymer seals, such as, but not limited to, silicone rubber, natural rubber, styrene butadiene rubber, ethylene-propylene copolymers, polychloroprene, polyacrylate, polybutadiene, polyurethane, styrene butadiene, and the like, and combinations thereof. For example, in certain embodiments, the seal is a septum pierceable by a needle, syringe, or cannula. The seal may also provide convenient access to a sample in the container, as well as a protective barrier that overlies the opening of the container. In some instances, the seal is a removable seal, such as a threaded or snap-on cap or other suitable sealing element that can be applied to the opening of the container. For instance, a threaded cap can be screwed over the opening before or after a sample has been added to the container.

As used herein, a “tissue disrupting device” is a device that causes cellular damage or injury. The tissue disrupting device may be configured to form a wound in a dermal location of a subject, e.g., according to any of the methods described herein, In some cases, the device may apply to a dermal location one or more of, e.g., ultrasound, radio frequency (RF), laser, ultraviolet energy, infrared energy, or mechanical disruption. Suitable tissue disrupting devices include, but are not limited to, surgical instruments (e.g., scalpels, lancets, etc.), needles, microneedles (e.g., a Dermaroller®), lasers, etc. In certain embodiments, the devices include 1, 2, 3, 4, 5, 6, 7, 8, 9, or 10 skin-penetrating component(s) (e.g., a needle, a drill, a microauger, a tube comprising cutting teeth, a spoon bit, a wire, a fiber, a blade, a high-pressure fluid jet, a cryoprobe, a cryoneedle, an ultrasound needle, a multi-hole needle including one or more chemical agents, a microelectrode, and/or a vacuum, or any other component described herein) that can penetrate the skin simultaneously. In some cases, the tissue disrupting device is configured to administer or deliver an effective amount of a YAP inhibitor composition to a wound, e.g., a wound formed by the tissue disrupting device. In certain embodiments, the tissue disrupting device is configured to administer, e.g., inject, the YAP inhibitor composition to a topical dermal location or below a topical dermal location of the subject. The administration may be performed with any suitable mechanism or medium according to any of the embodiments described above such as, e.g., a needle, microneedle, gel, etc. In some cases, one or more portions of the tissue disrupting device contains an effective amount of a YAP inhibitor composition, In some cases, the tissue disrupting device includes one or more microneedles. In some cases, the tissue disrupting device includes an array of microneedles. In certain embodiments, the tissue disrupting device is a microneedling device including, e.g., the Dermaroller® or Dermapen®. In some cases, the tissue disrupting device is a laser, e.g., for practicing fractional laser resurfacing.

### Utility

The subject methods find use in applications involving wound healing including, e.g., clinical and research applications. In certain embodiments, the methods find use in postnatal wound healing or wound healing in adults. The methods may find use in any applications where a wound is intentionally, e.g., via surgery, or unintentionally created.

In certain embodiments, the subject methods find use in applications where it is desirable to reduce or prevent scarring. The subject methods may be applied to the treatment of all types of skin, including wound zones and eyes, where scarring is a possibility. In certain embodiments, the methods may be used to treat or prevent scarring of human skin resulting from burns, scalds, grazes, abrasions, cuts and other incisional wounds, surgery and pathological skin scarring conditions such as, e.g., Dupuytren's disease, and the conditions of fibrotic dermal scarring, hypertrophic scarring, keloid scarring and corneal and other ocular tissue scarring.

The subject methods further find use in applications for promoting hair growth. The subject methods may find use in applications where increased hair growth in a particular dermal location is desired, e.g., a region of substantial hair loss. In certain embodiments, the methods find use in treating hair loss and conditions involving hair loss as a side effect. The methods may be used to treat hair loss from a variety of conditions, such as, but not limited to hormonal changes during pregnancy and childbirth, disease (hyper- and hypo-thyroidism, lupus, trichotillomania), medication, chemotherapy, dietary deficiencies, stress, alopecia, trauma, radiotherapy, iron deficiency or other nutritional deficiencies, autoimmune diseases and fungal infection. In certain embodiments, the subject methods find use in treating a subject for alopecia.

The following example(s) islare offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Whey & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Example 1

### Inhibiting Engrailed-1 Activation in Mechanosensitive Fibroblasts Yields Wound Regeneration without Scarring

### A. Materials & Methods:

### Mice

Transgenic mouse strains: En-1^{Cre} (En1^{tm2(cre)Wrst}/J), En-1^{Cre-ERT} (En1^{tm7(cre/ESR1)Alj}/J), R26^{mTmG} (Gt(ROSA)26Sor^{tm4(ACTB-tdTomato,-EGFP)Luo}/J), Ai6 (B6.Cg-Gt(ROSA)26Sor^{tm6(CAG-ZsGreen1)Hze}/J), and NOD-SCID (NOD.CB17-Prkdc^{scid}/J).

Mice were bred and maintained at the Stanford University Comparative Medicine Pavilion in accordance with Stanford APLAC guidelines (APLAC-11048). Mice were housed and bred under the care of the Department of Comparative Medicine in the Veterinary Service Center (VSC). En1^{Cre}, En1^{Cre-ERT}, Gt(ROSA)26Sor^{tm4(ACTB-tdTomato,-EGFP)Luo} (R26^{mTmG}) and B6.Cg-Gt(ROSA)26Sor^{tm6(CAG-ZsGreen1)Hze} (Ai6) mouse strains were obtained from Jackson Laboratories. En1^{Cre} and En1^{Cre-ERT} mice were crossed with Ai6 and mT/mG reporter mice to trace all EPFs and postnatal EPFs, respectively, as defined in vivo by their GFP positivity.

Transgenic mouse strains were validated by tissue collection and genotyping of each individual animal. The following primers were used: for En-1^{Cre} and En-1^{Cre-ERR} mice (band size Cre: 102 bp, internal positive control: 74 bp) Cre forward 5′-GCG GIC TGG CAG TAA AAA CTA TC-3′, Cre reverse 5′-GTG AAA CAG CAT TGC TGT CAC TT-3′, IPC forward 5′-CAC GTG GGC TCC AGC ATT-3′, IPC reverse 5′-TCA CCA GTC ATT TCT GCC TTT G-3′; for R26^{mTmG} (band size mutant: 140 bp, wt: 96 bp) mutant reverse 5′-GTT ATG TAA CGC GGA ACT CCA-3′, wt reverse 5′-CAG GAC AAC GCC CAC ACA-3′, common forward 5′-CTT CCC TCG TGA TCT GCA AC-3′. The PCR conditions were; 94° C. for 10 mins, 94° C. for 30 sec, 56° C. for 1:30 min, 72° C. for 1.5 min, repeat 35 cycles, 72° C. for 8 mins. Ai6 and R26^{VT2/GK3} mice were genotyped by visualization for green fluorescence under ultraviolet illumination.

### Harvesting Dermal Fibroblasts

Mice were euthanized by CO₂ narcosis and cervical dislocation, the dorsal fur was clipped, a depilatory cream was applied topically to the dorsum for 30 seconds. Next, the dorsal skin was harvested using dissecting scissors by separation along fascial planes, the subcutaneous fat was trimmed with a scalpel, and the skin was rinsed in betadine, followed by 5 rinses in cold PBS. To achieve a cell suspension, the harvested skin was finely minced using sharp scissors, enzymatically digested (Liberase DL, 0.5 mg/mL, 1 hour), and filtered through a 40 μm nylon mesh. ENFs and EPFs were isolated from En-1^{Cre};R26^{mTmG} mice (En-1 lineage-negative cells, mTomato⁺; En-1 lineage-positive cells, GFP⁺) via a previously reported FACS strategy. Briefly, a lineage gate (Lin) for hematopoietic (CD45, Ter-119), endothelial (CD31, Tie2), and epithelial (CD326, CD324) cell markers was used as a negative gate to isolate fibroblasts (Lin⁻), which were sorted into ENFs (Tornato⁺GFP⁻Lin⁻) and EPFs (Tomato⁻GFP+ Lin⁻). To isolate ENF subpopulations, dorsal skin cells were harvested from P1 En-1^{Cre,}Ai6 mice (En-1 lineage-negative cells, no fluorescence; En-1 lineage-positive cells, GFP⁺) via mechanical and enzymatic digestion as described above. Cells were then stained for the aforementioned lineage markers, in addition to CD26, Dlk1, and Sca1 in order to derive ENFs of the papillary dermis (Lin⁻CD26⁺Dlk1⁻Sca1⁻), reticular dermis (Lin⁻CD26⁻Dlk⁺Sca1⁻), and hypodermis (Lin⁻CD26⁻Dlk1^{+/-}Sca1⁺). Cells were resuspended in FACS buffer and DAPI before FACS analysis.

### Cell Engraftment

One-day old (P1) En-1^{Cre};B26^{mTmG} and En-1^{Cre}; Ai6 mice were used to isolate ENFs and EPFs for both engraftment and in vitro studies for the following three reasons. First, P1 mice are known to heal with a similar scarring outcome as older P60 mice. Second, neonatal mouse skin is more cellular than juvenile or adult mouse skin, so fewer mice can be sacrificed to derive the high cell numbers required for successful engraftment. Finally, it is observed that P1 cells retain higher viability after engraftment than P60 cells, Recipient mice (P60 C57BL/6 or R26^{mTmG}) were anesthetized (2% isofluorane), their dorsal hair was removed using depilatory cream, and their skin was prepped with alcohol wipes. Injection sites (two 6 mm circular regions per mouse at the level of the scapulae, roughly 8 mm lateral to midline) were marked with a skin marker, and fibroblasts were injected intradermally (100,000 cells per mouse; n=3 mice each receiving ENFs, ENF subpopulations, or EPFs) around the border of each region. Cells were allowed to engraft for 48 h, after which separate, 6 mm full-thickness excisional wounds (see below) were made at each marked injection site, such that the engrafted cells were now located at the wound edge.

### Dorsal Excisional Wounding

P60 En-1^{Cre};R26^{mTmG}, En-1^{Cre};Ai6, and En-1^{Cre-ERT};Ai6 mice were used for cutaneous wound healing experiments in accordance with well-established protocols. Briefly, mice were anesthetized (2% isofiuorane), their dorsal hair was removed with depilatory cream, and the dorsal skin was prepped with alcohol wipes. Next, two 6 mm full-thickness circular wounds were placed through the panniculus carnosus on the dorsum of each animal at the same level, roughly 6 mm below the ears and 4 mm lateral to the midline. The wounds were then stented open by 12 mm diameter silicone rings secured around the wound perimeter with glue and 8 simple interrupted Ethilon 6-0 sutures (Ethicon). For mice receiving mechanotransduction inhibitor, 30 μL of Verteporfin (1 mg/mL) was injected locally into the wound base; PBS was injected into wounds for vehicle controls. Post-operative analgesia was accomplished with buprenorphine 0.05 mg/kg every four hours for three doses, and then as indicated. Dressings were changed every other day under anesthesia. All wounds were fully re-epithelialized by post-operative day (POD) 14, at which time the wound and surrounding skin (used as unwounded control) were harvested and processed for histology. Induction of En-1^{Cre-ERT};Ai6 mice was achieved by 5 consecutive days of intraperitoneal tamoxifen injections (90% corn oil/ethanol v/v; 200 mg/kg body weight) prior to wounding. In all experiments, a minimum of 3 mice with 2 wounds each was used for each treatment group.

### Mechanical Loading of Wounds

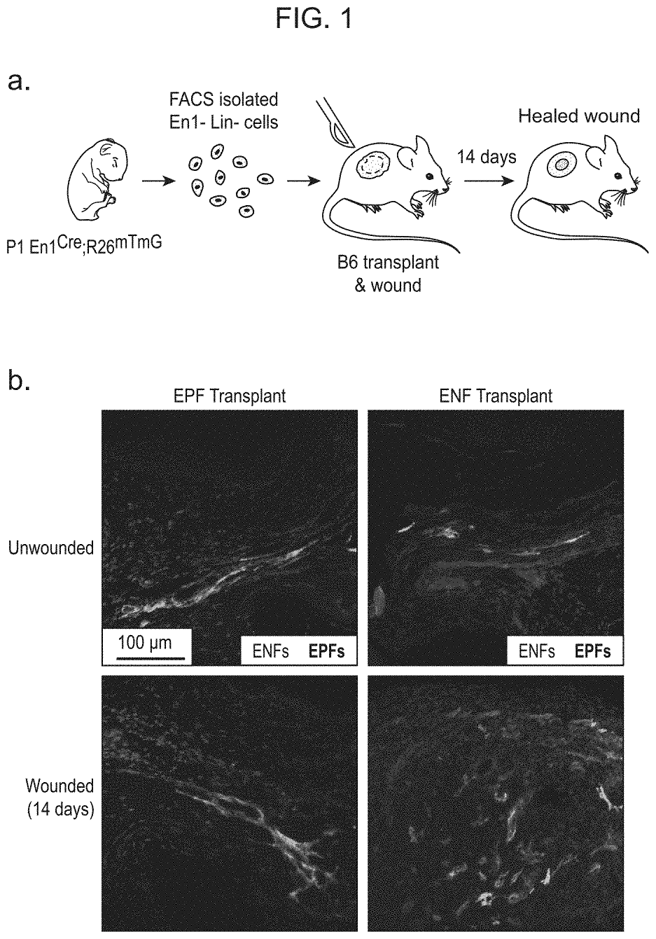

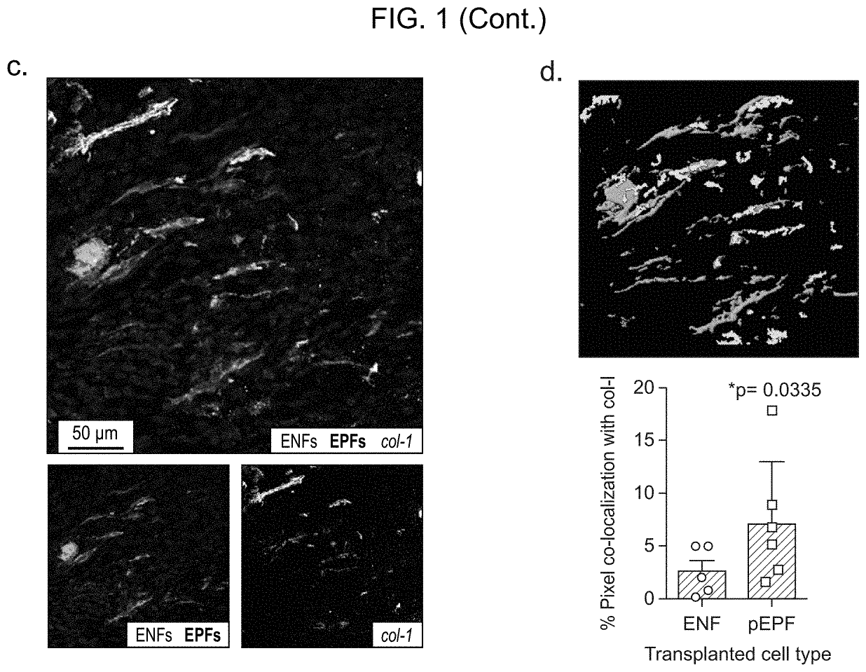

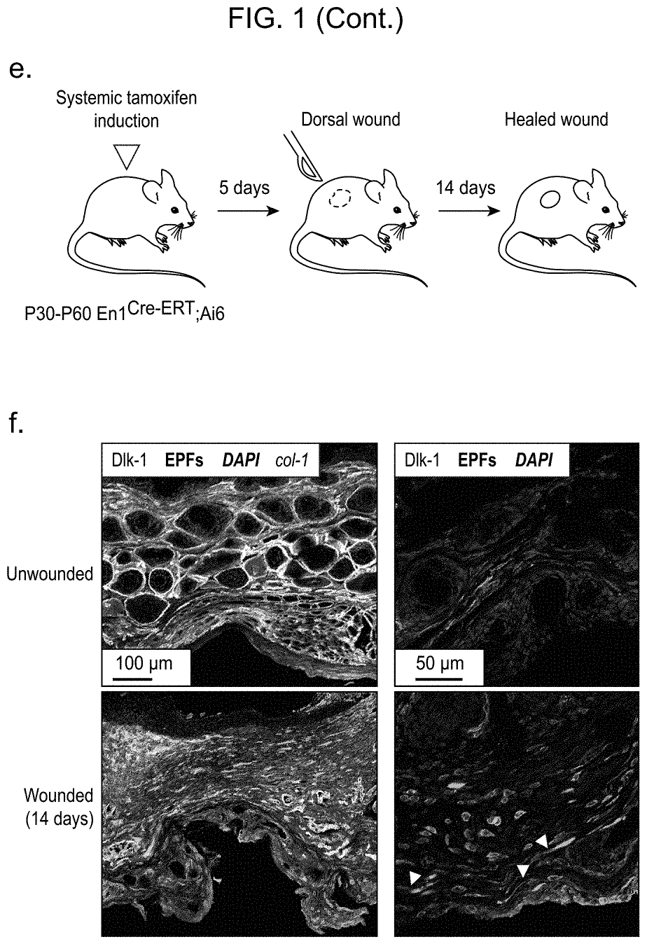

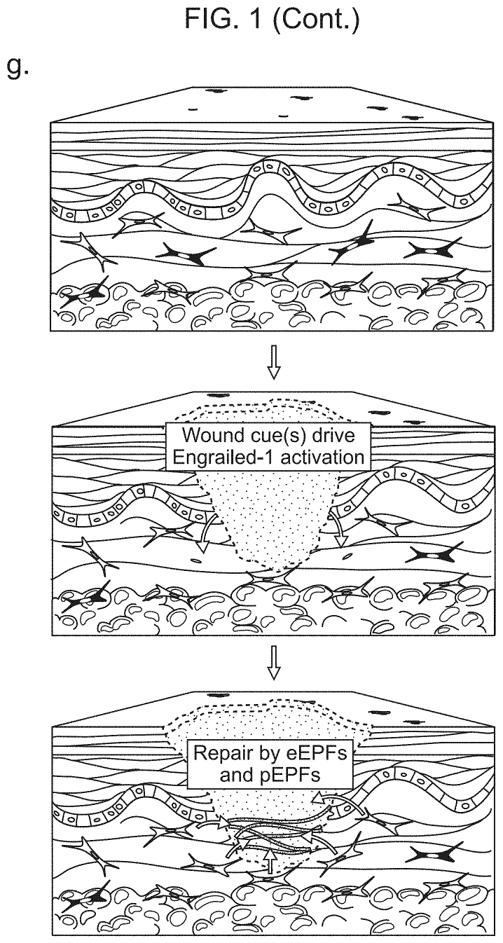

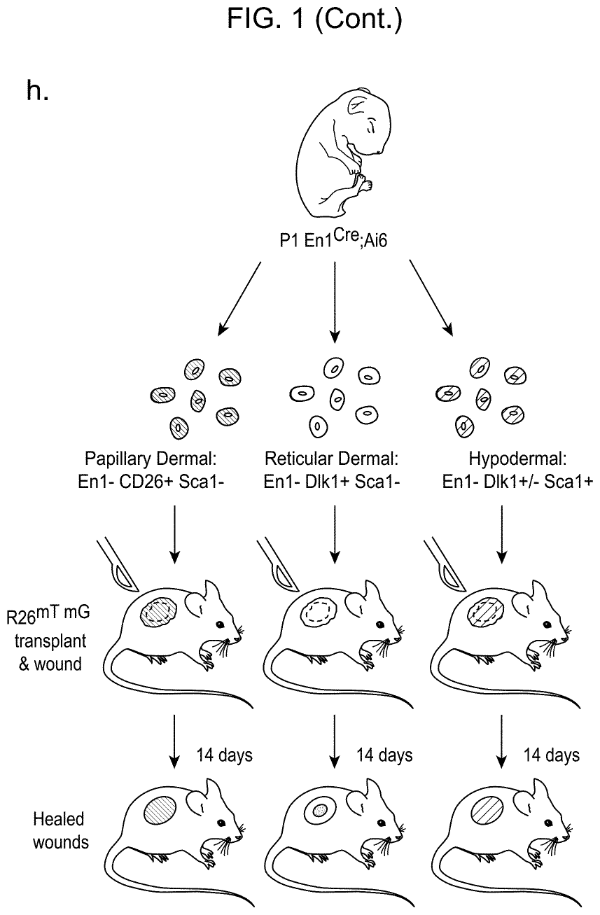

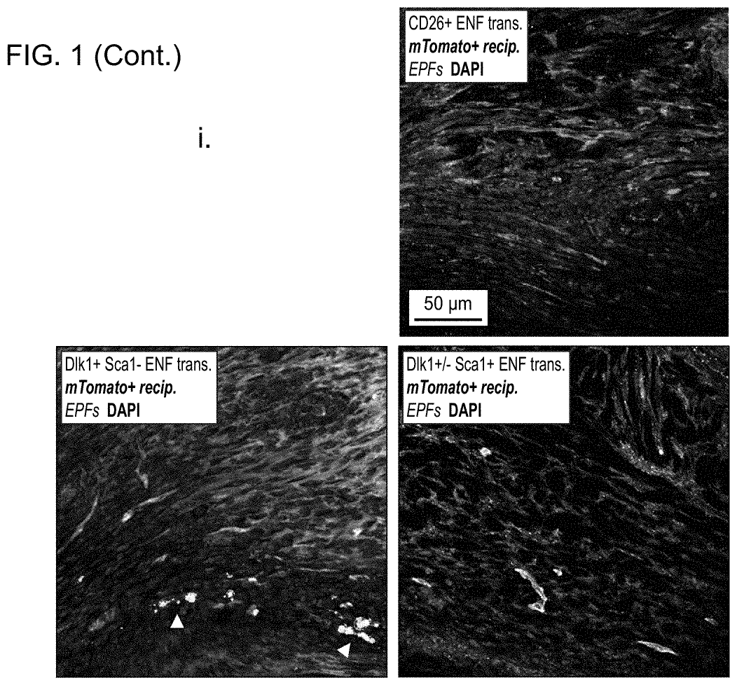

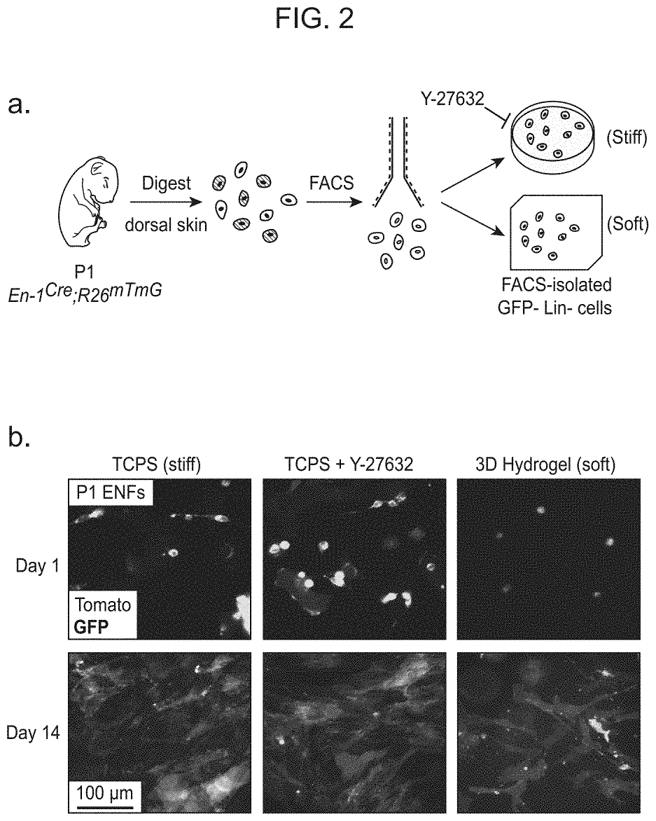

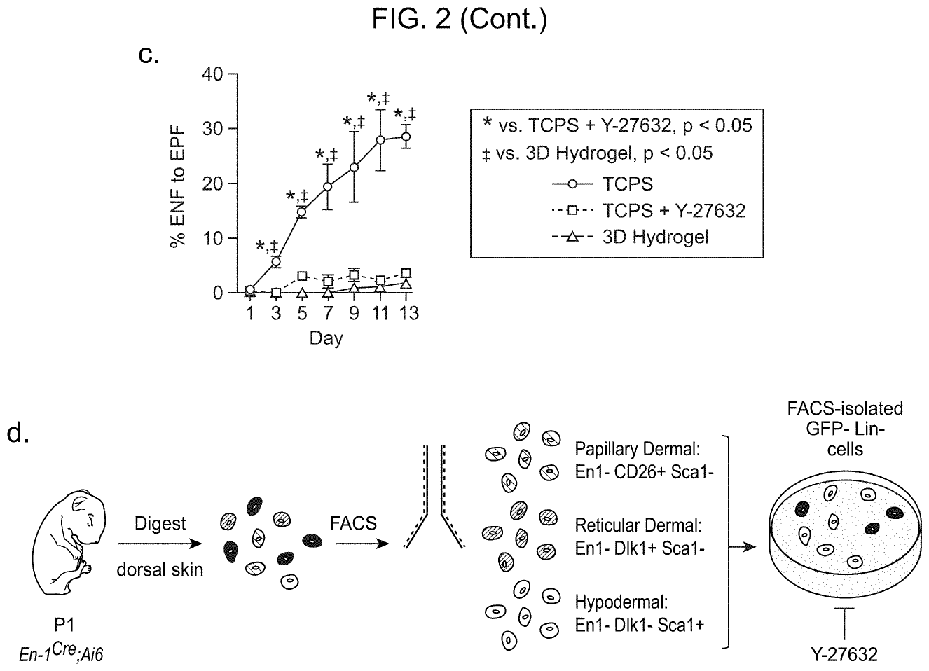

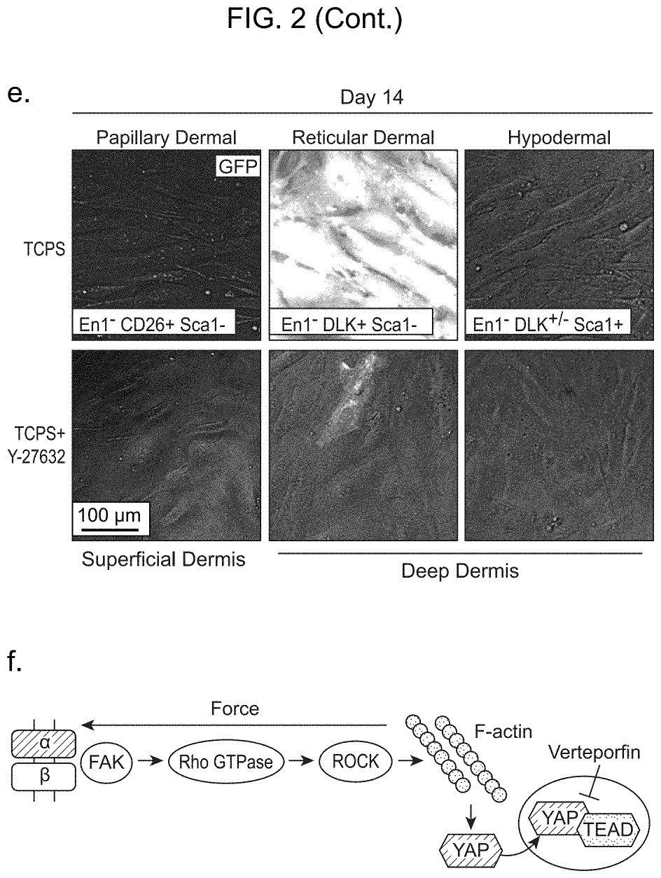

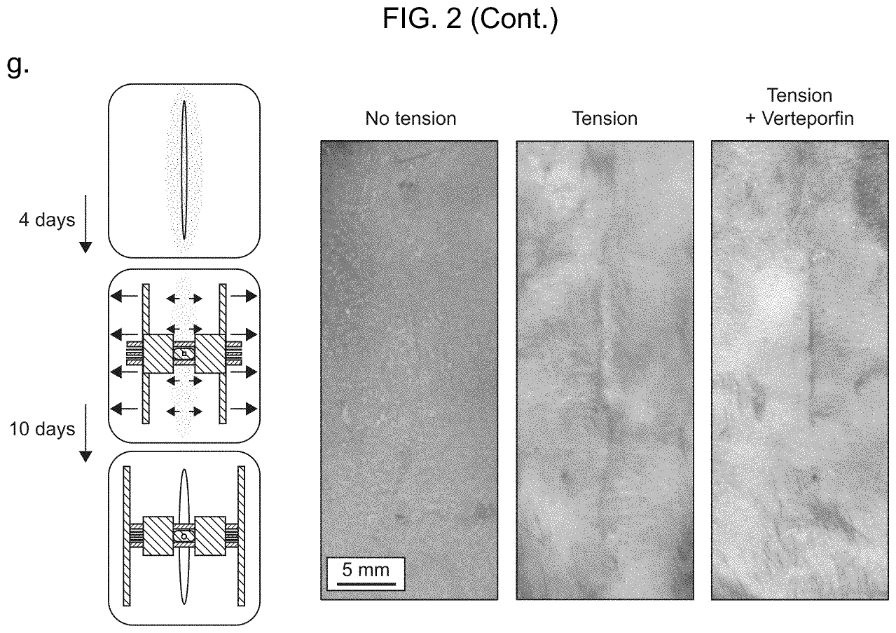

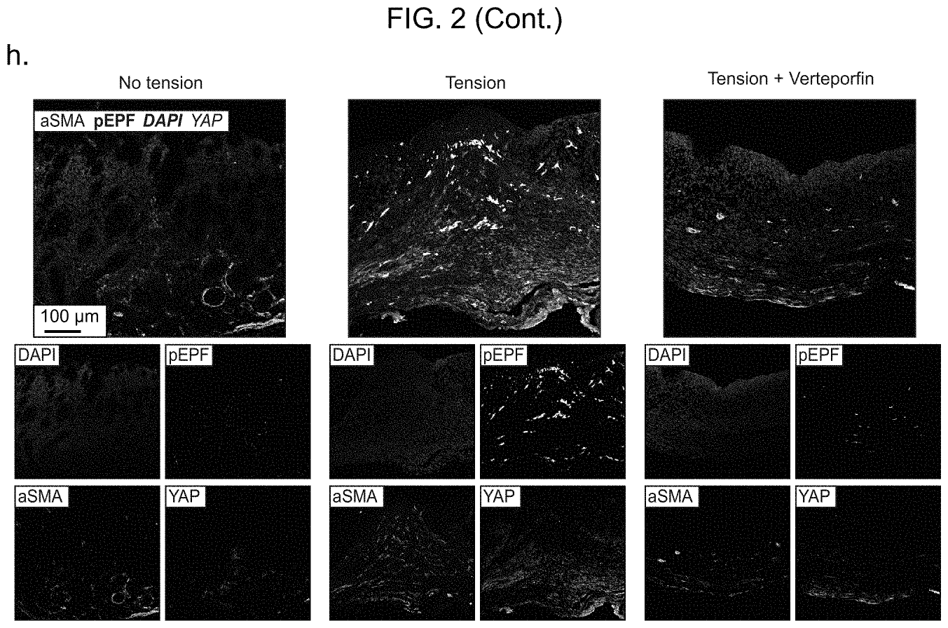

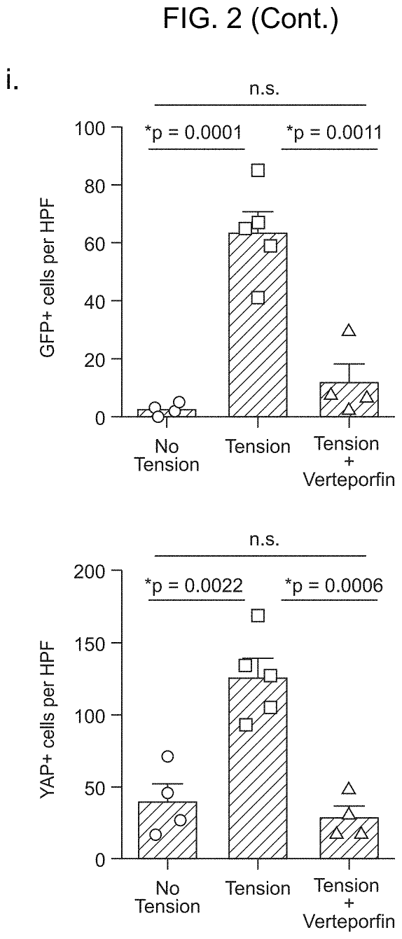

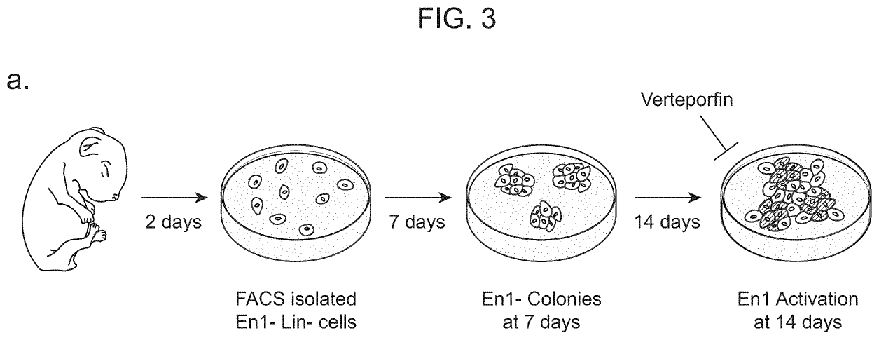

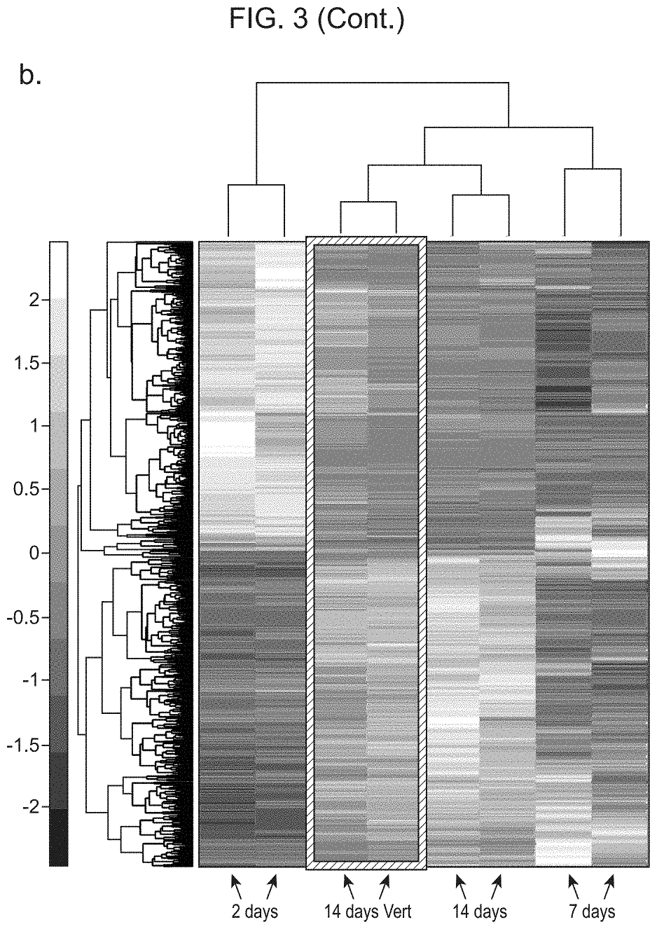

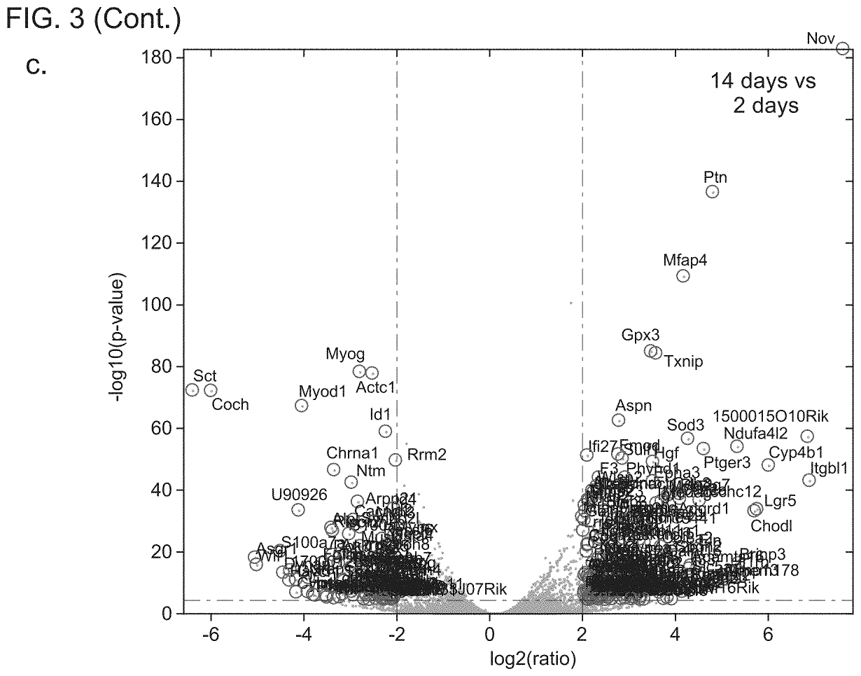

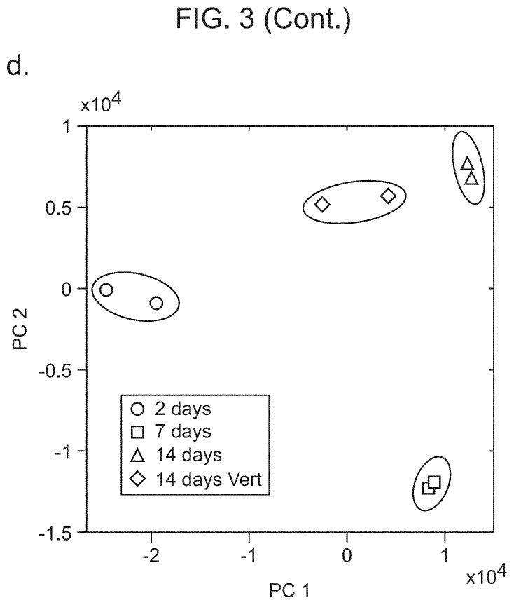

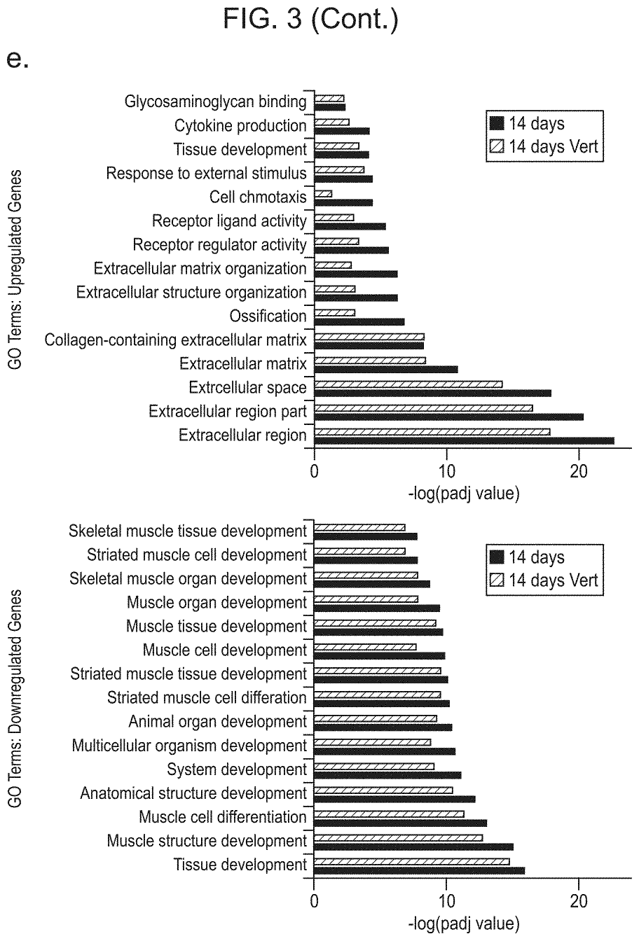

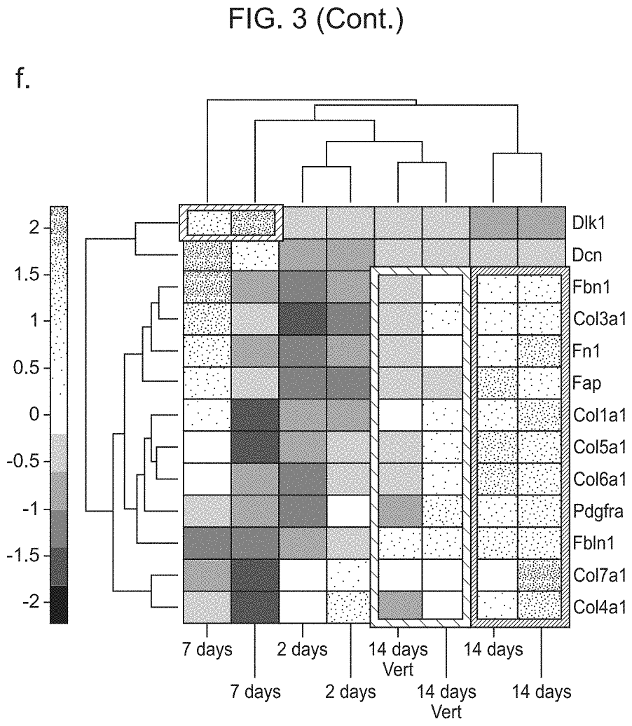

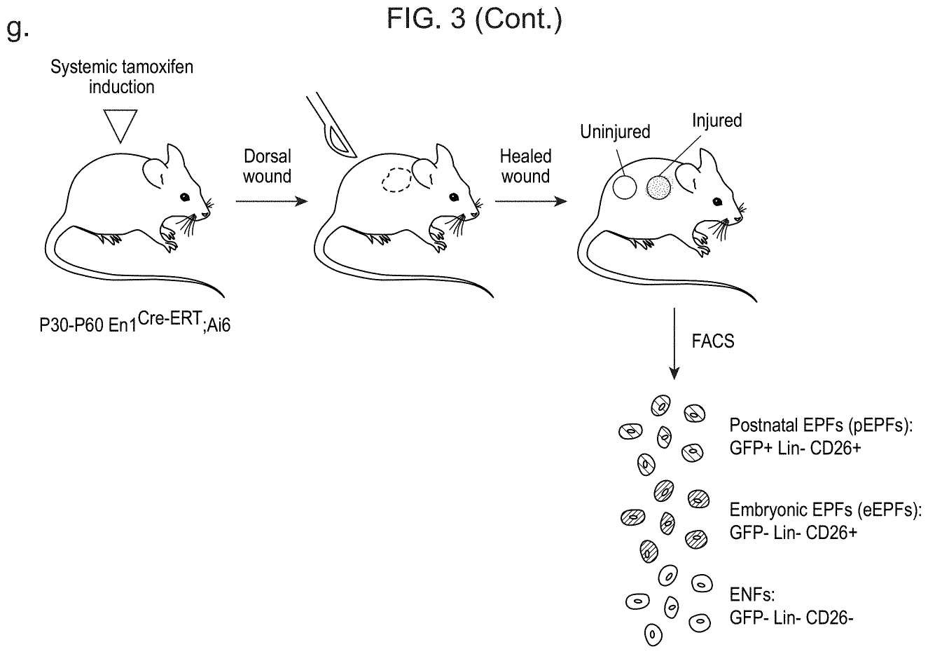

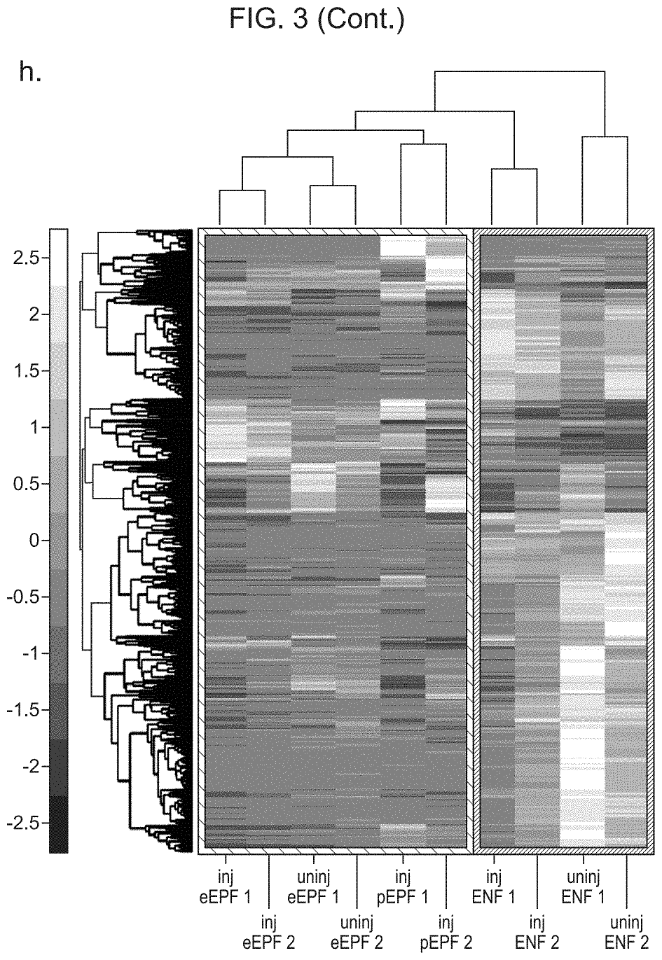

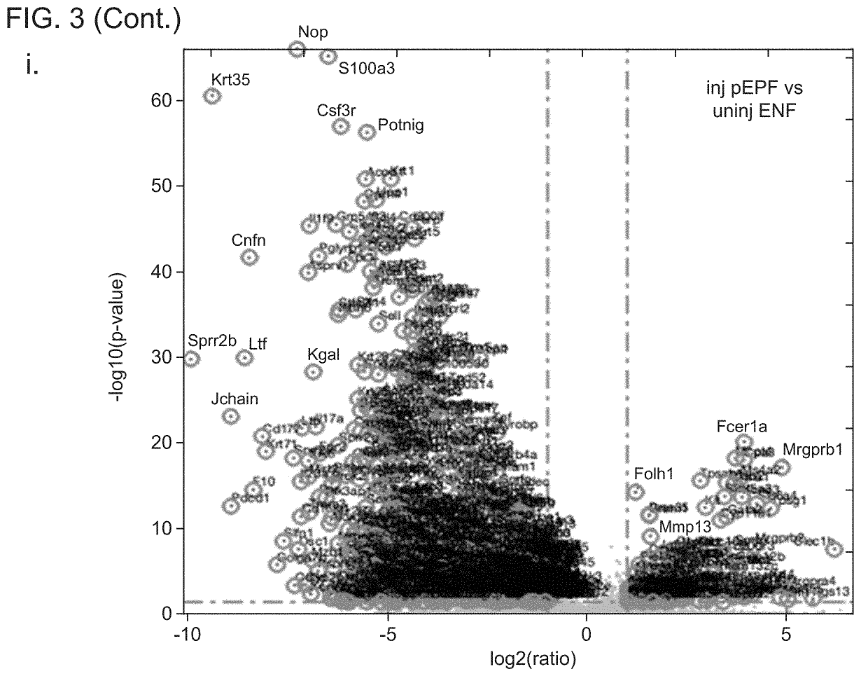

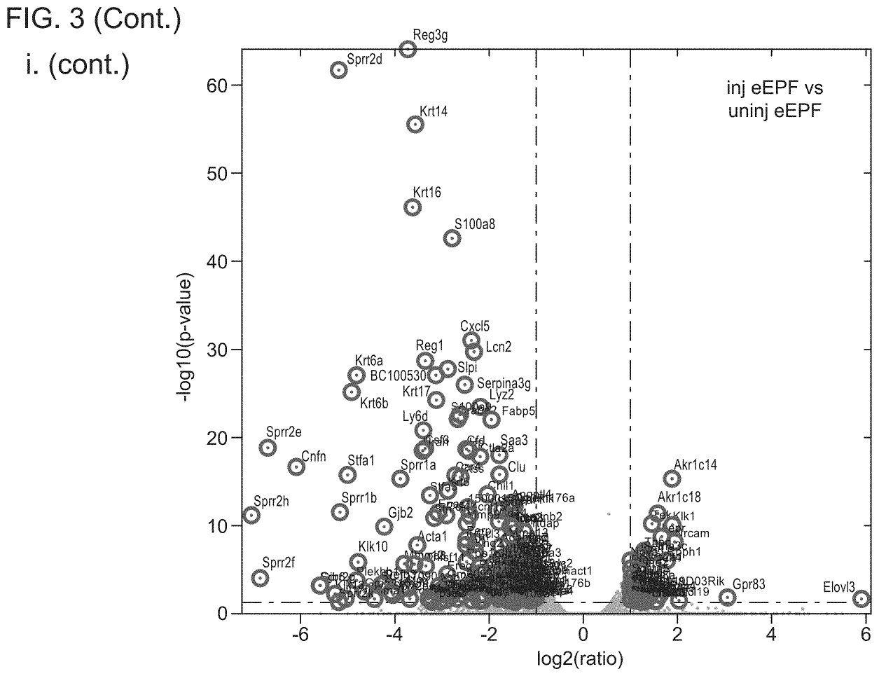

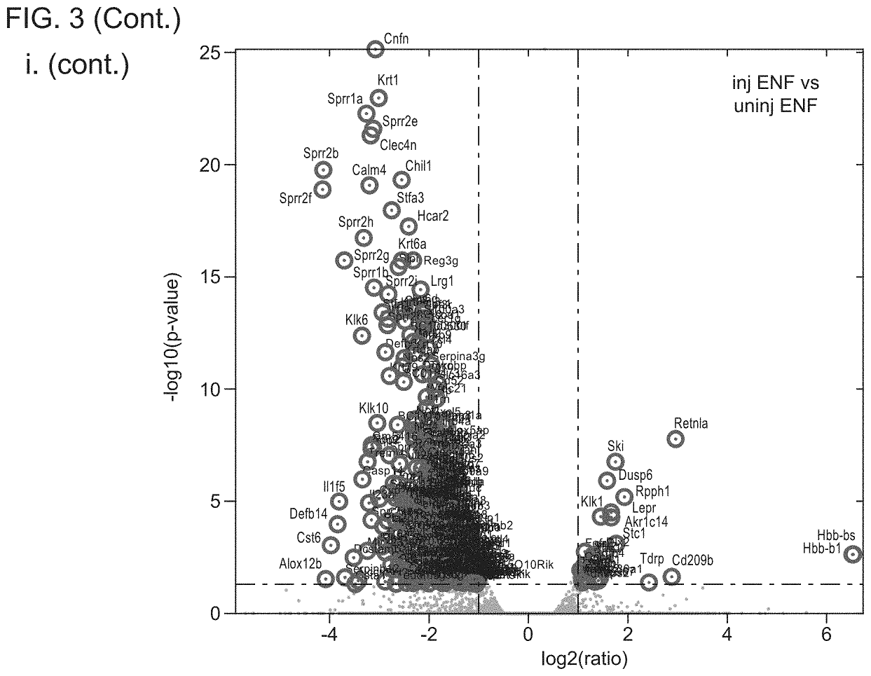

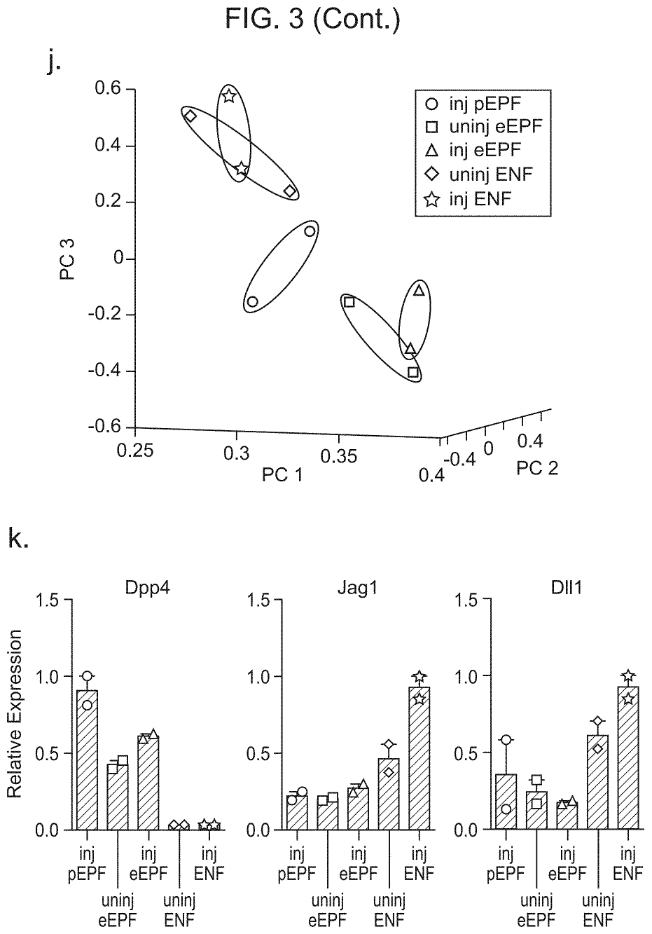

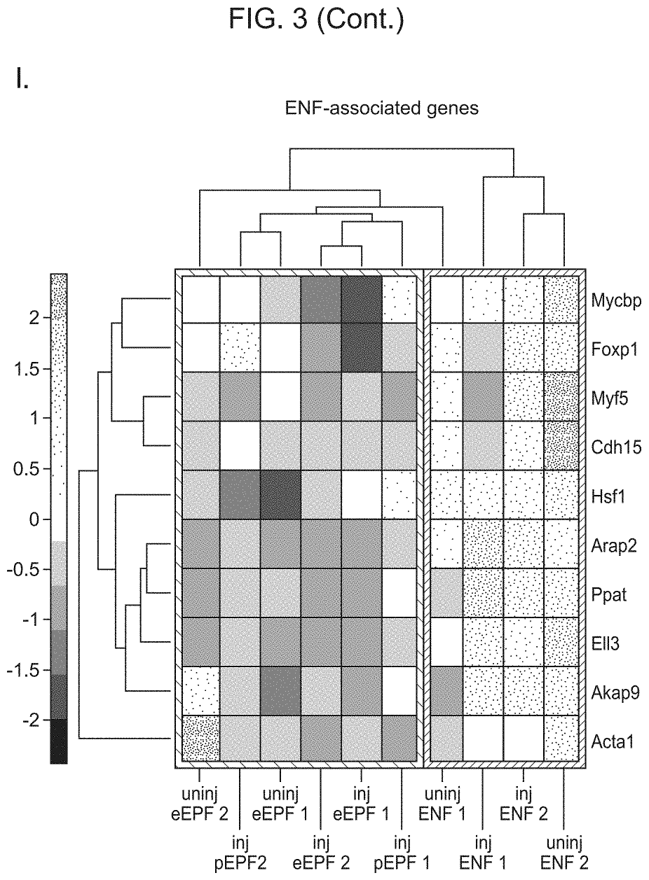

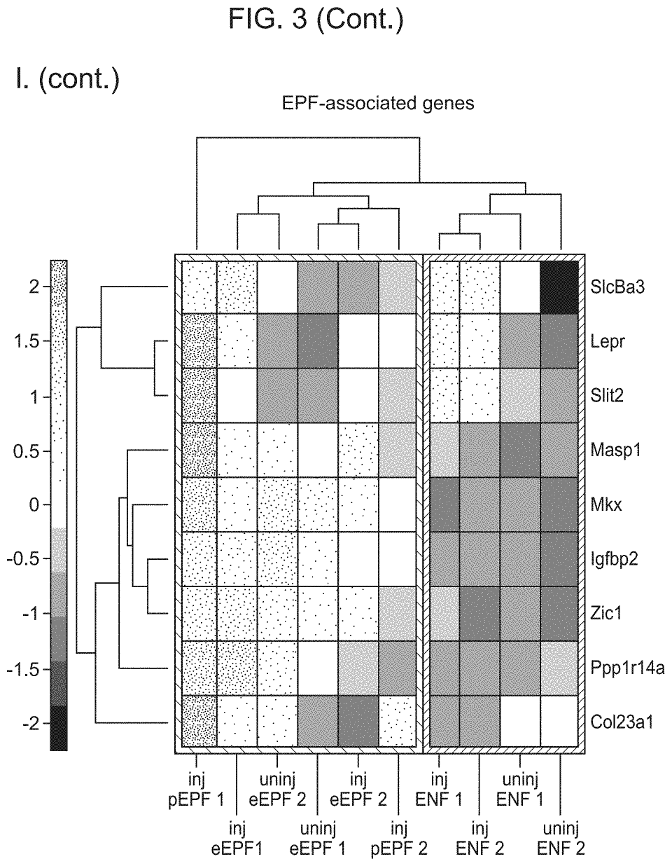

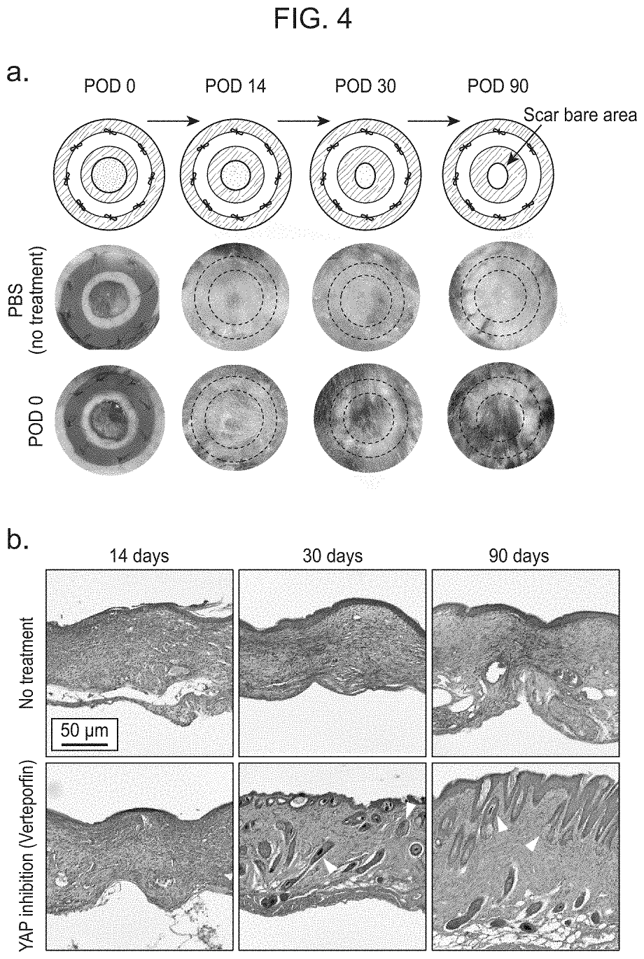

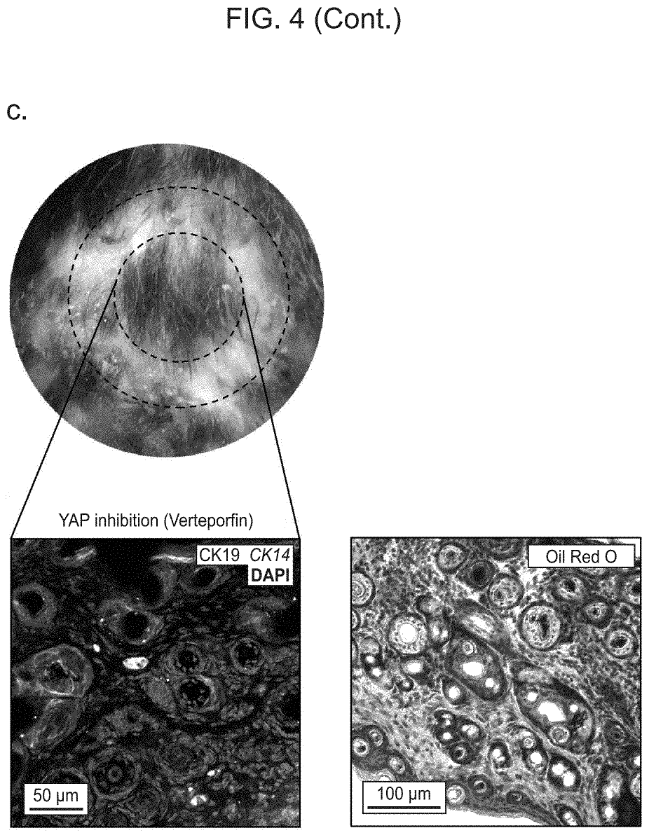

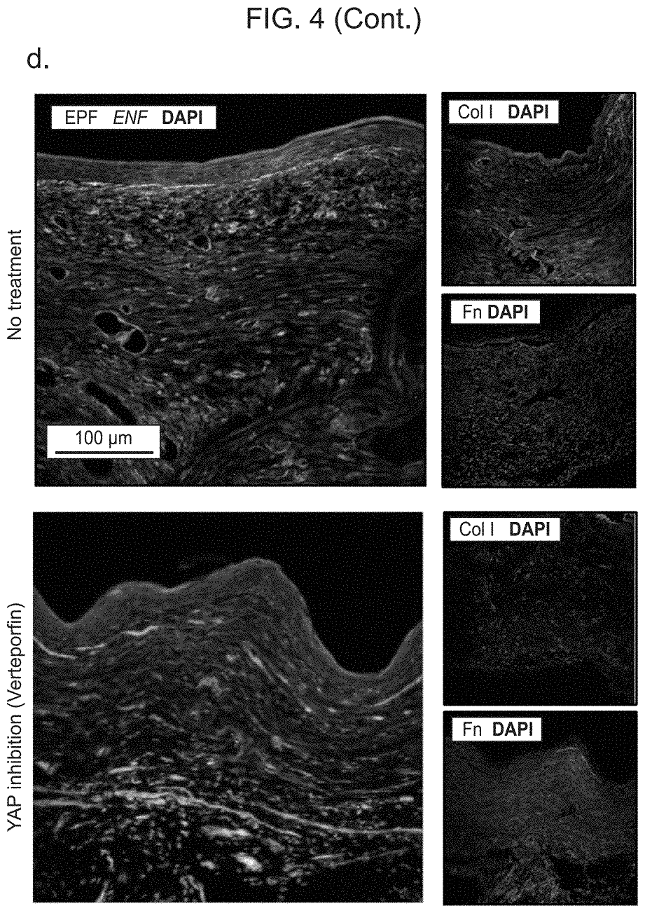

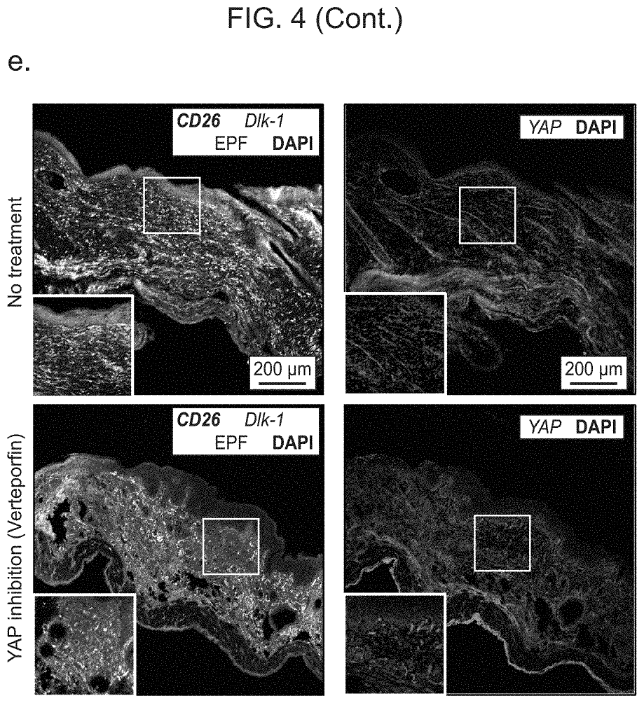

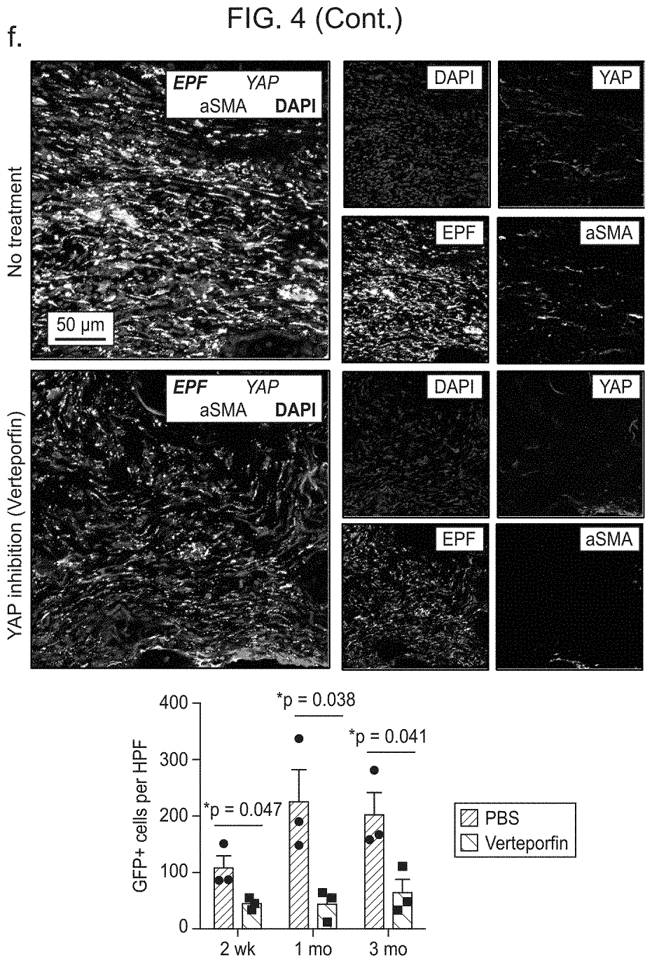

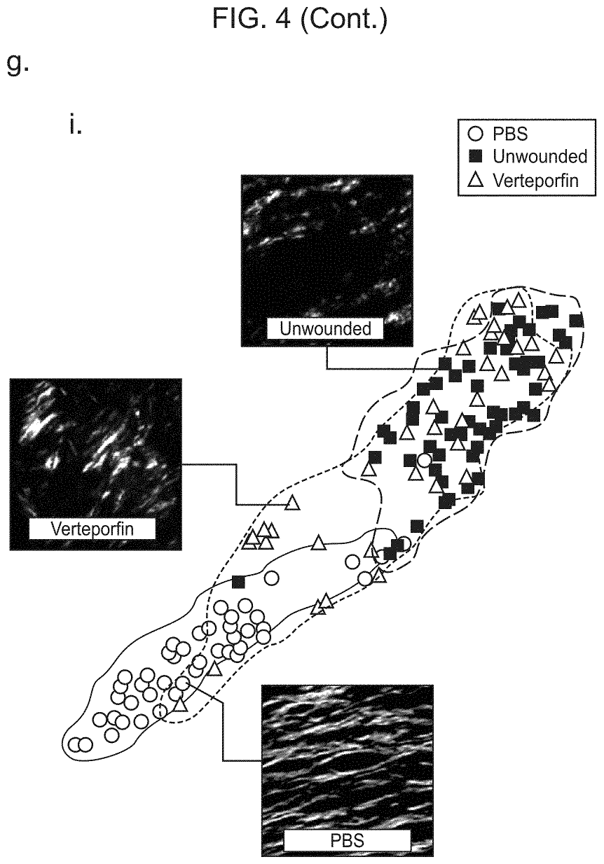

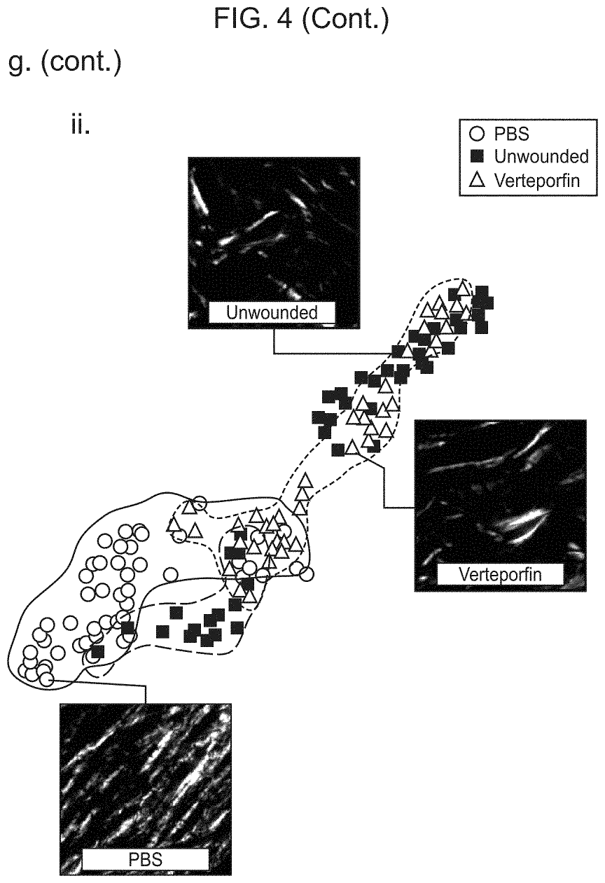

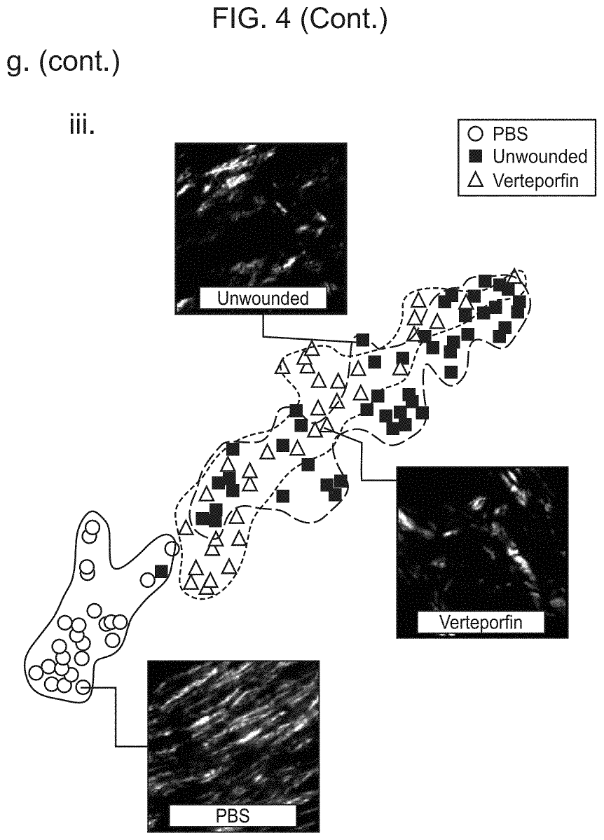

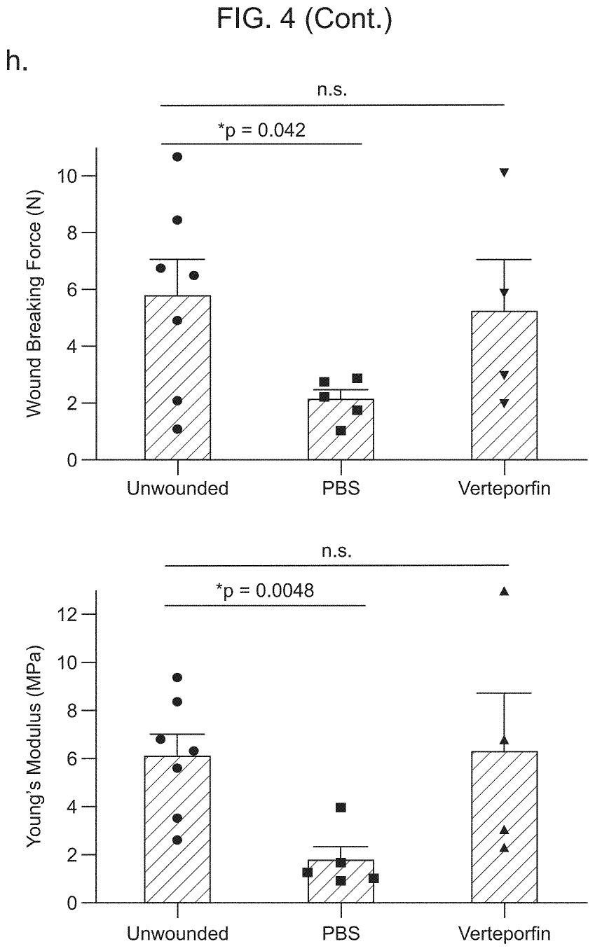

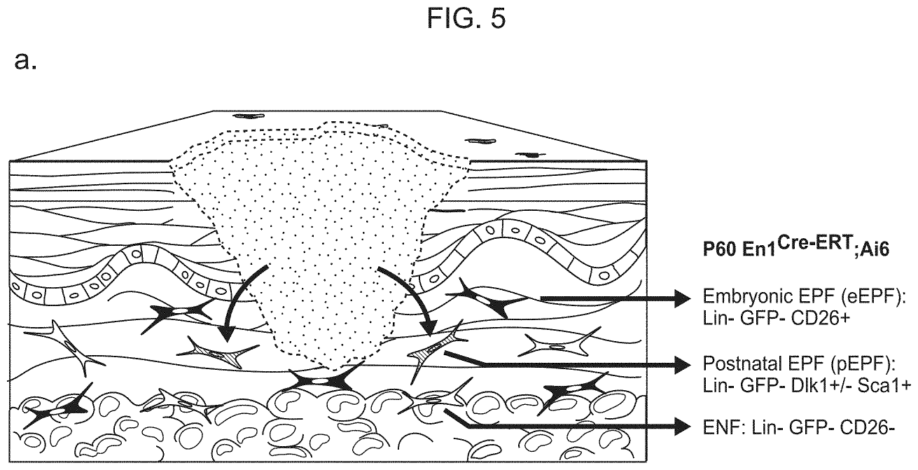

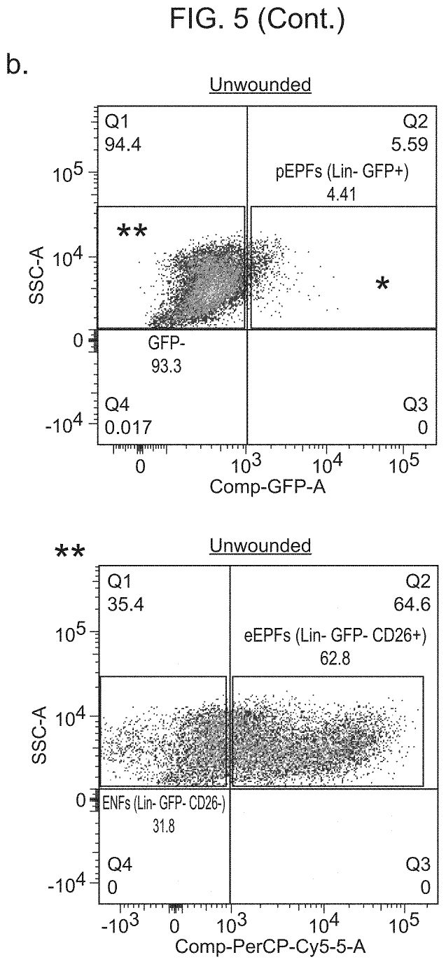

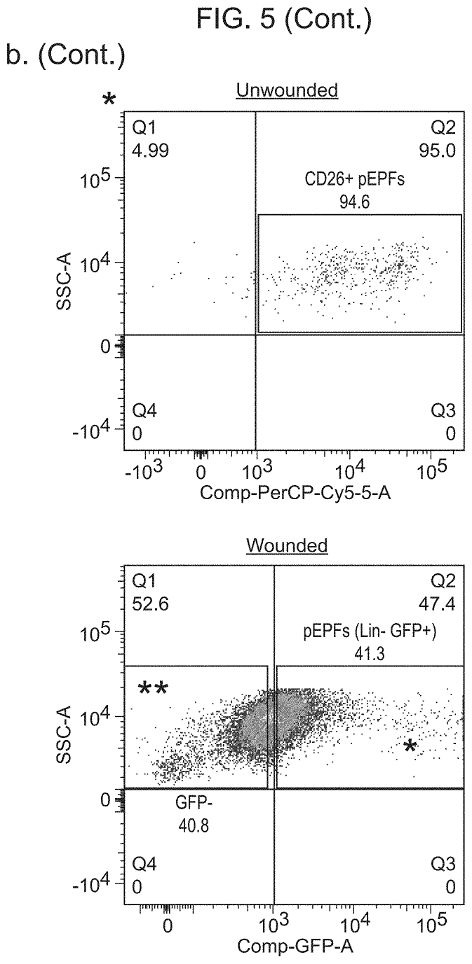

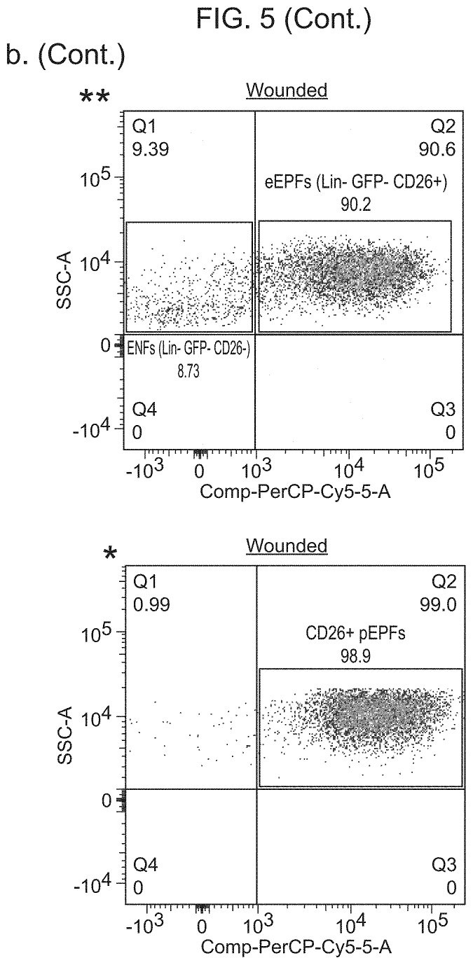

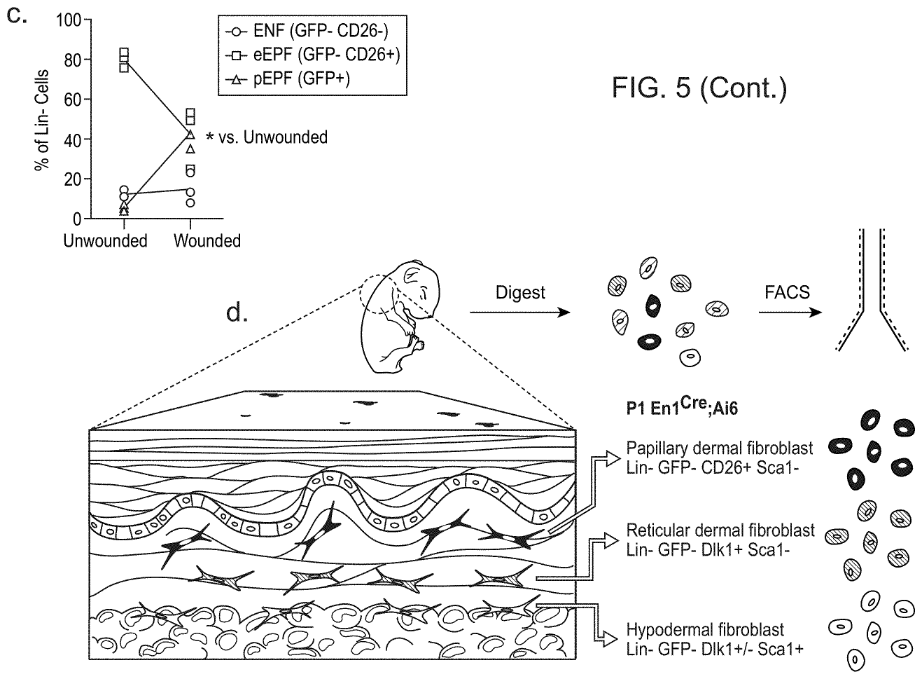

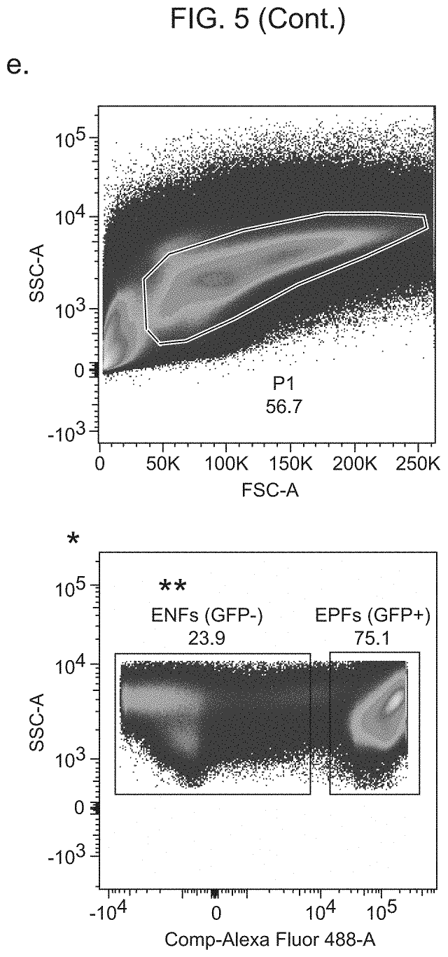

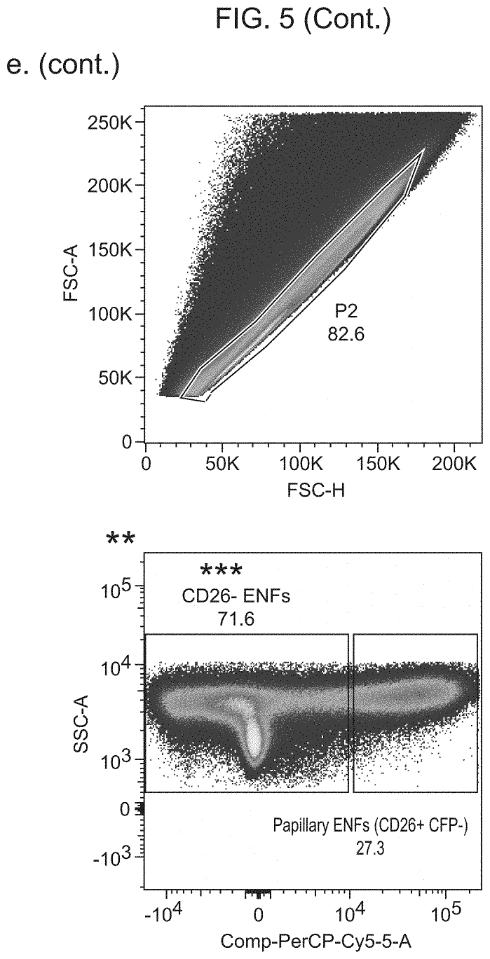

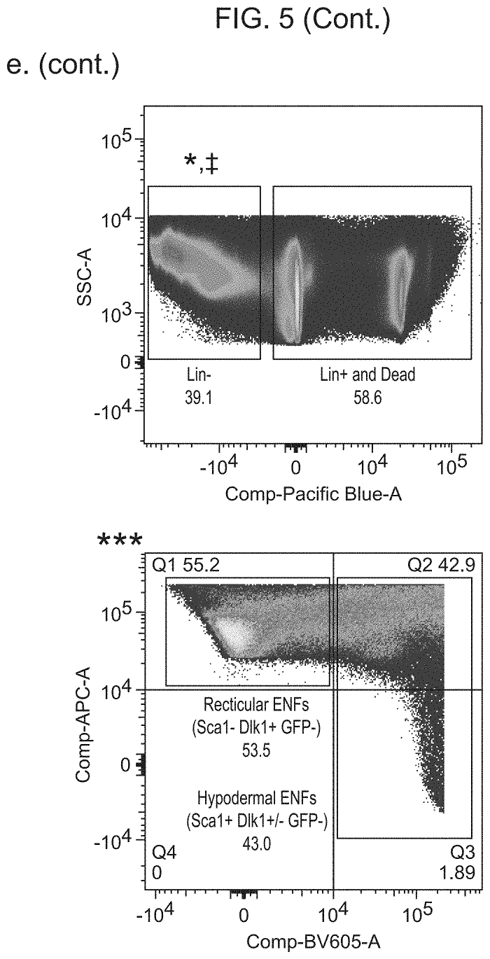

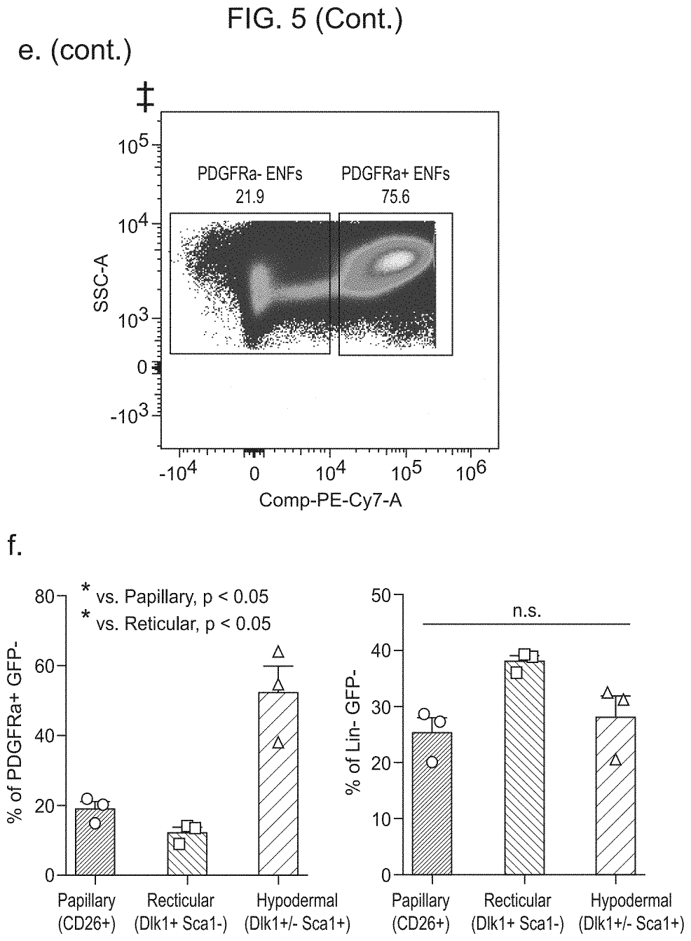

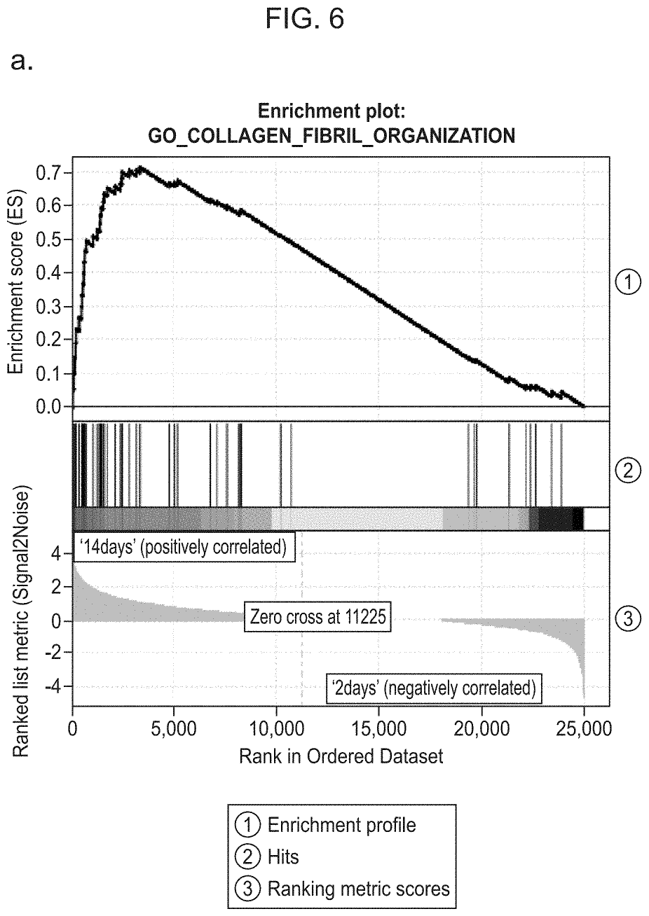

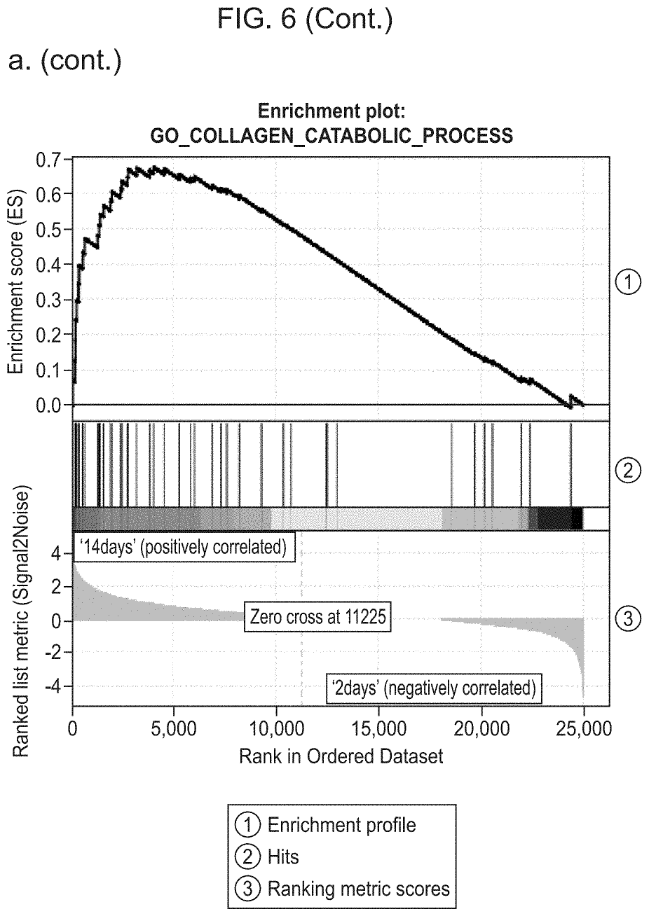

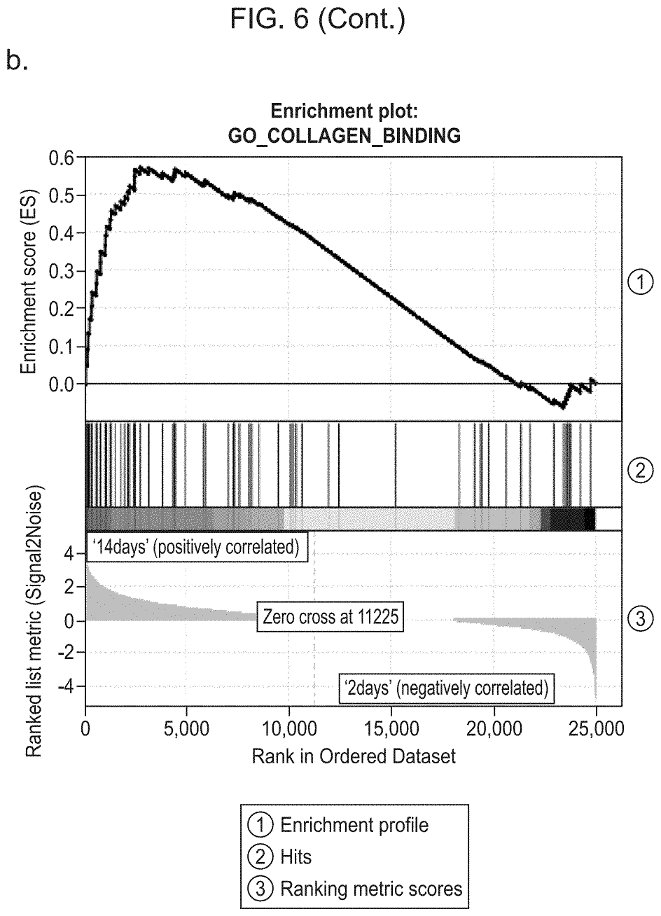

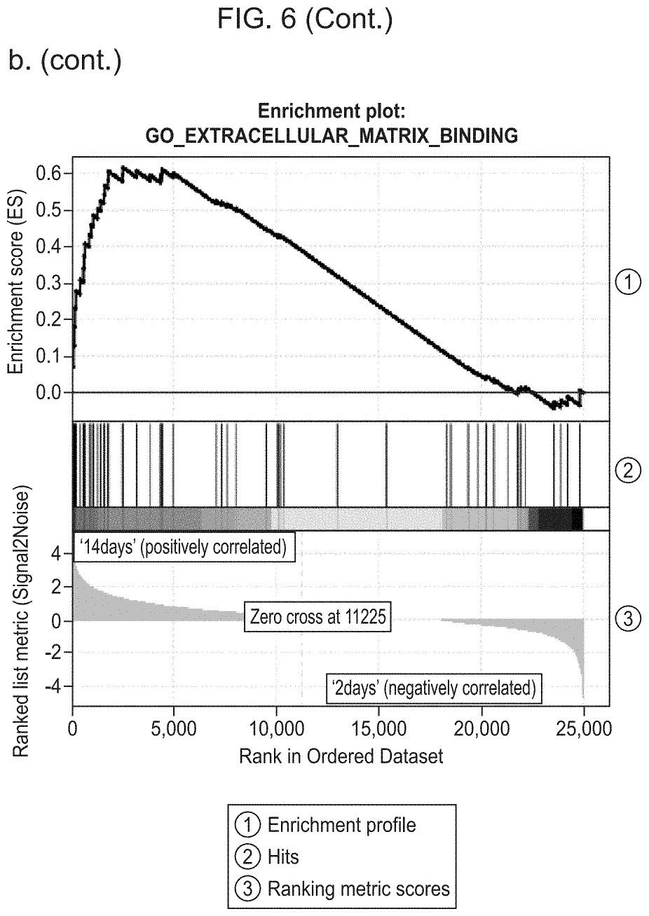

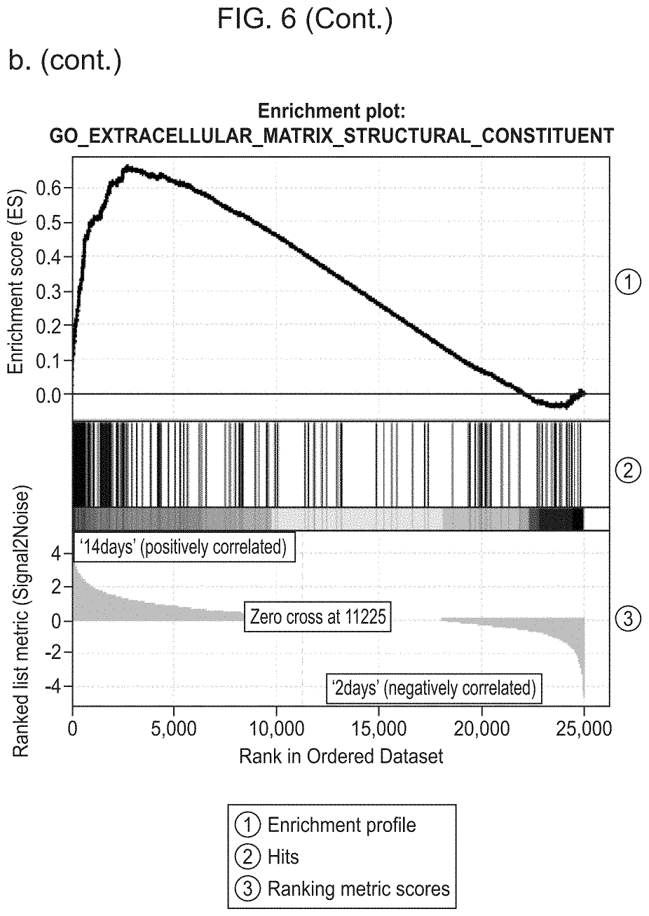

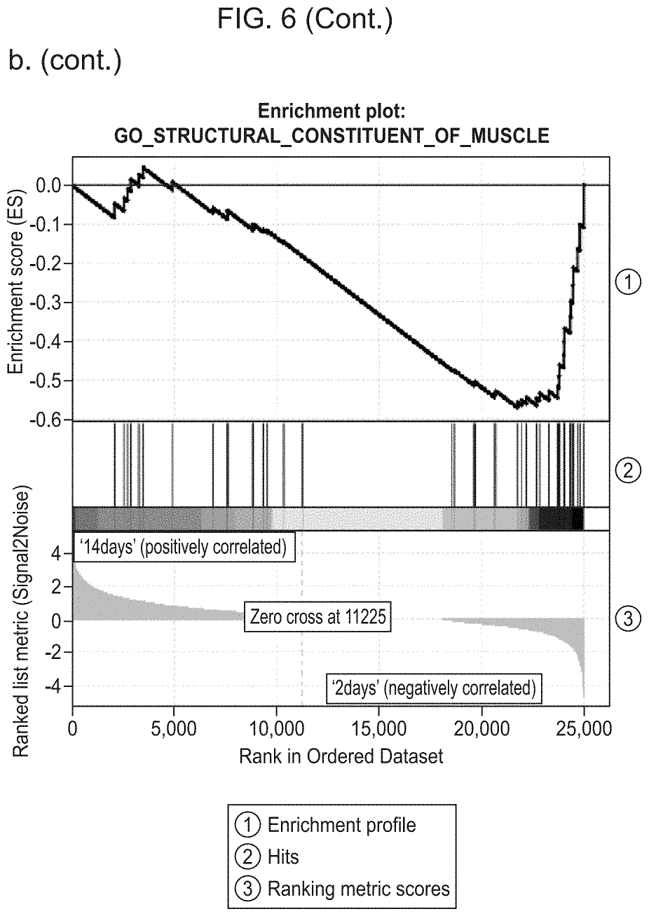

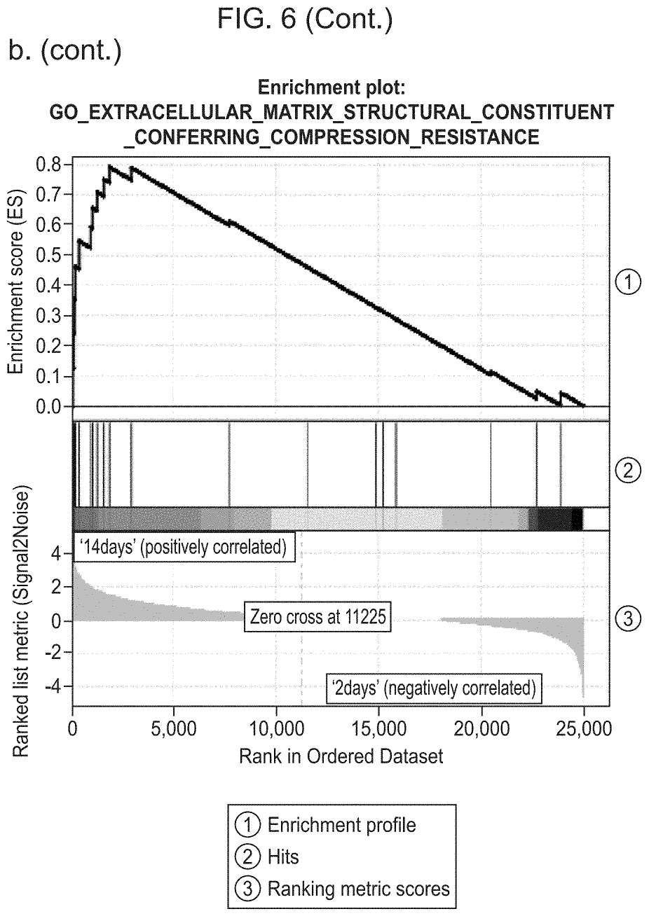

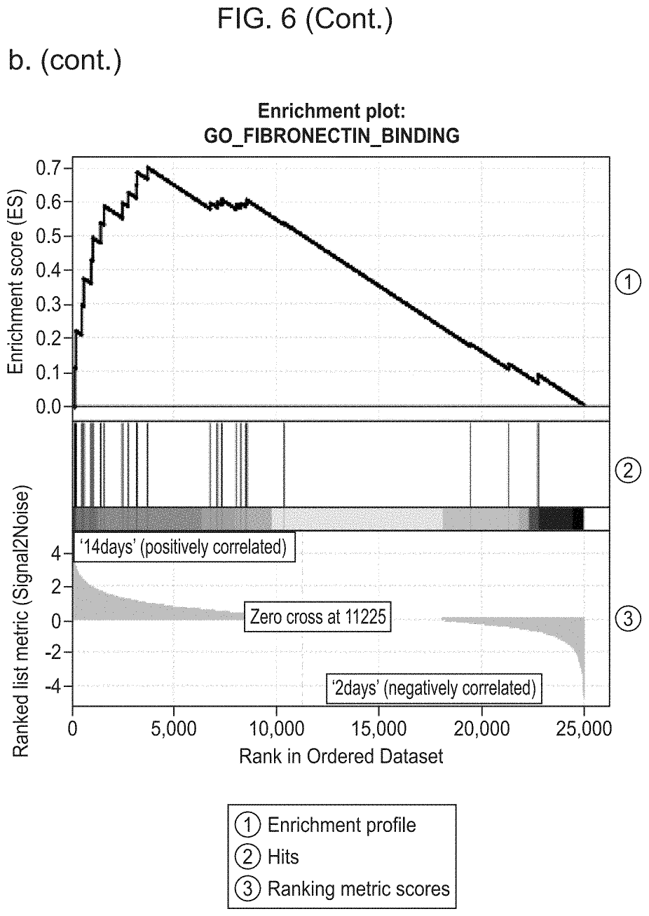

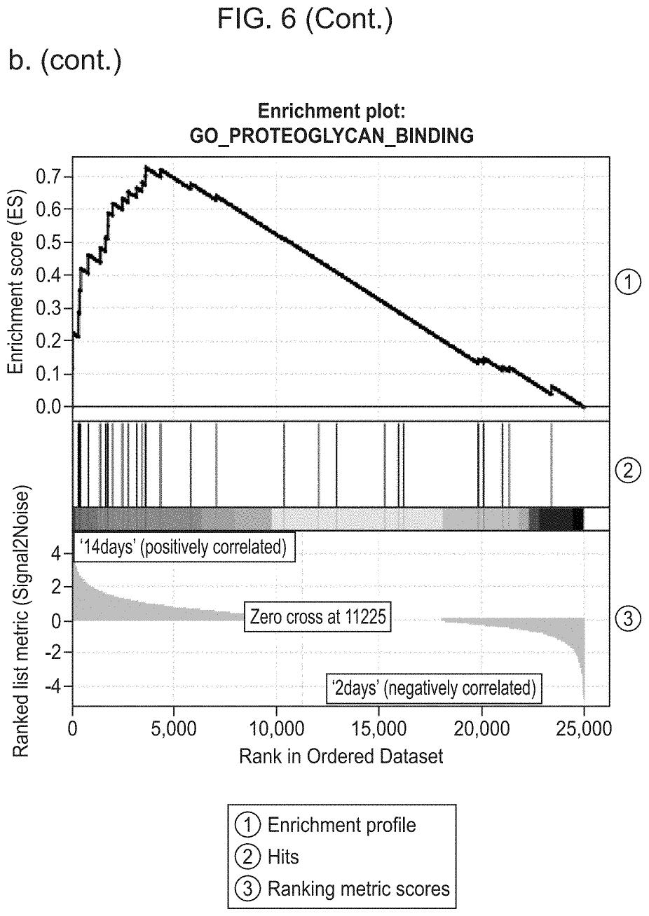

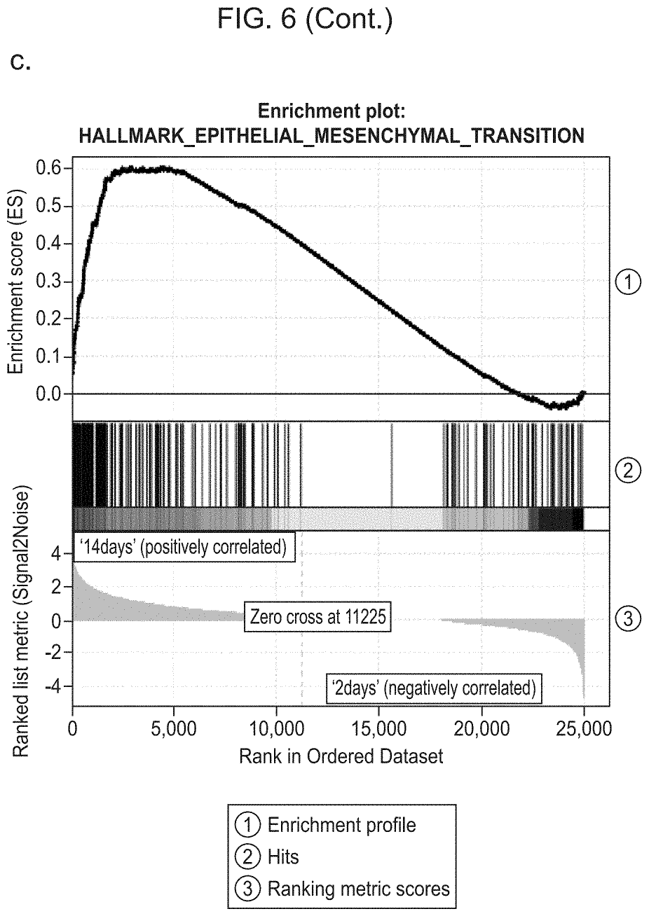

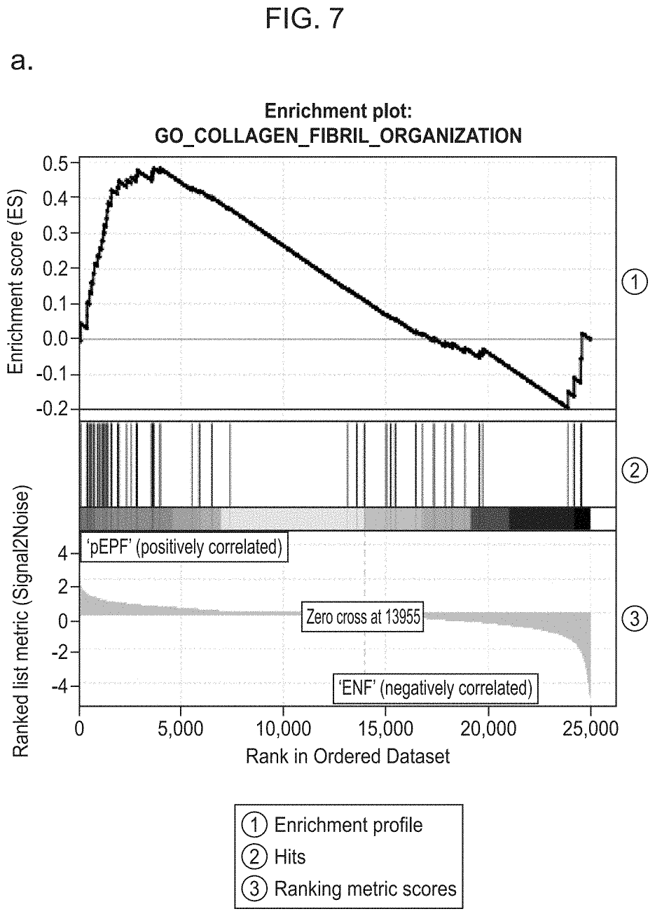

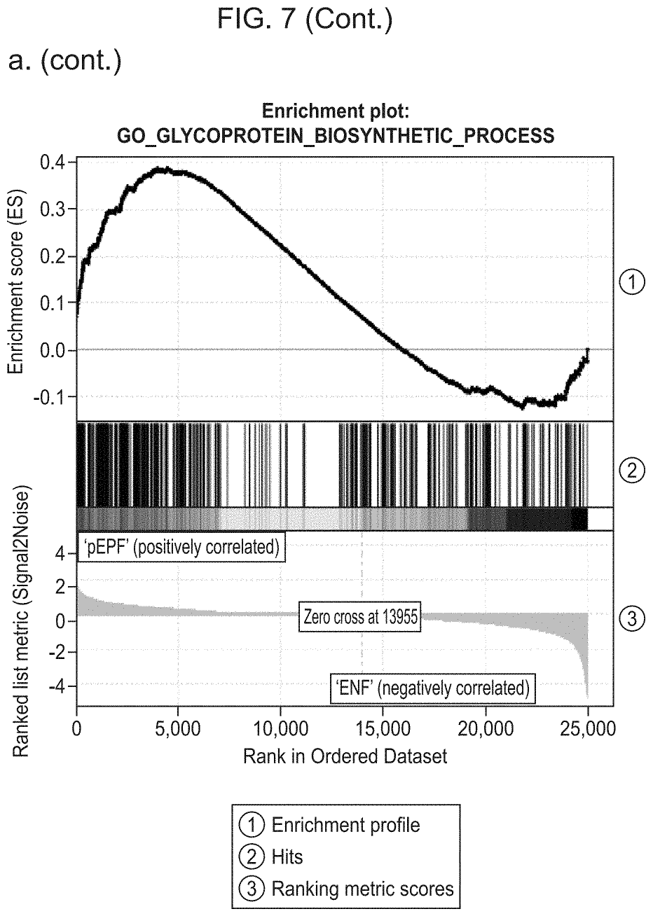

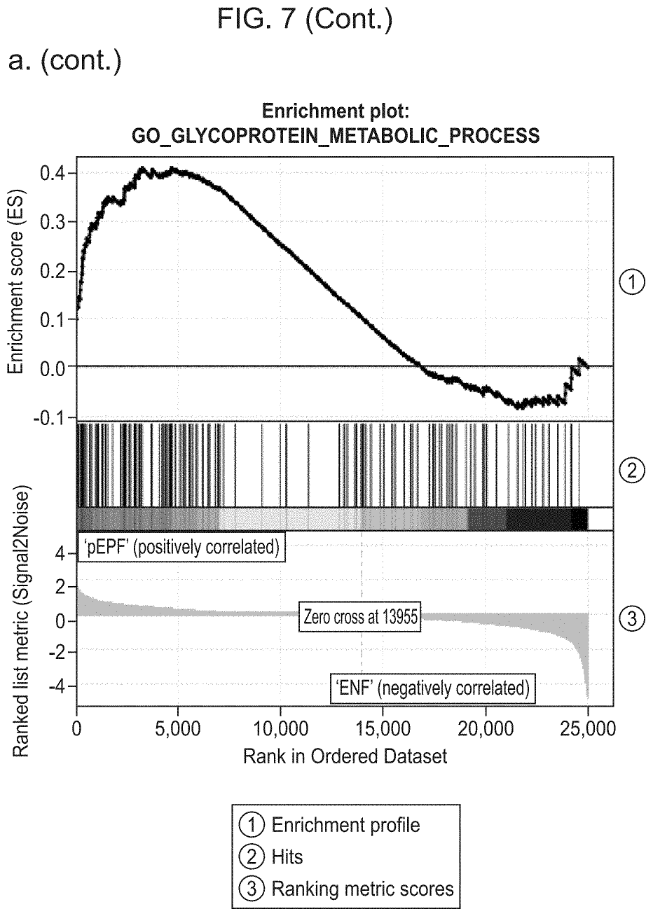

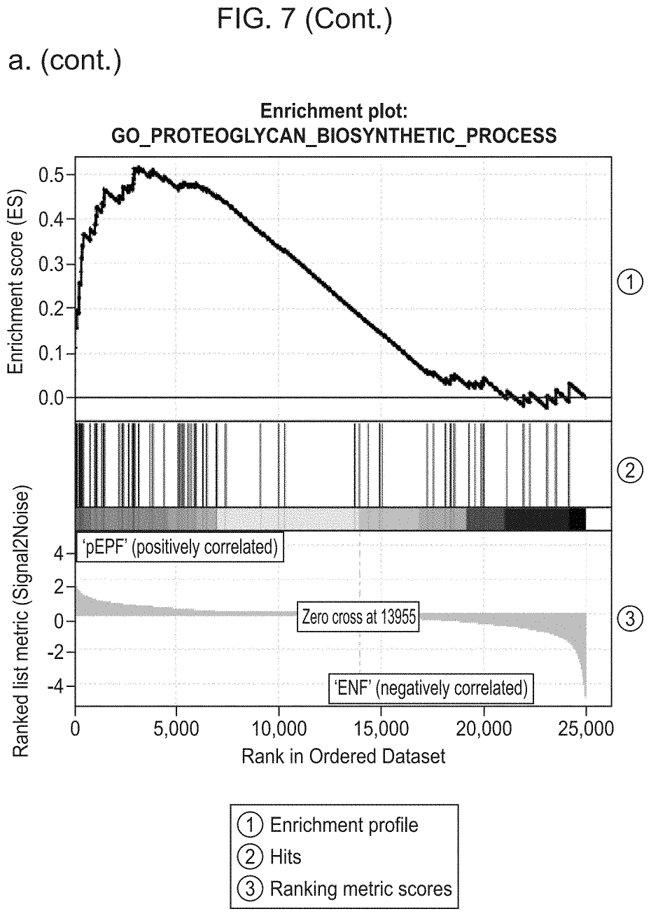

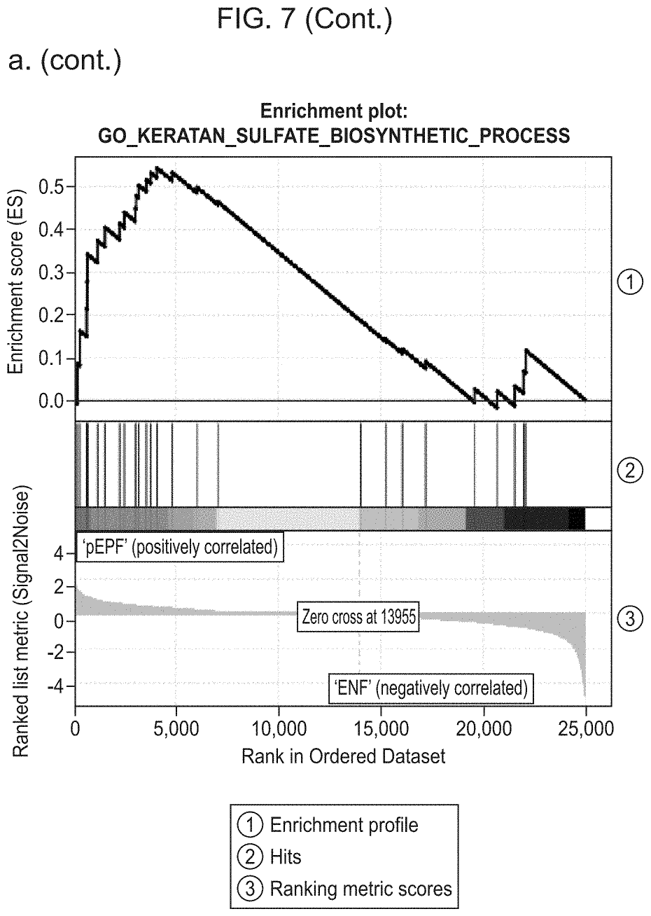

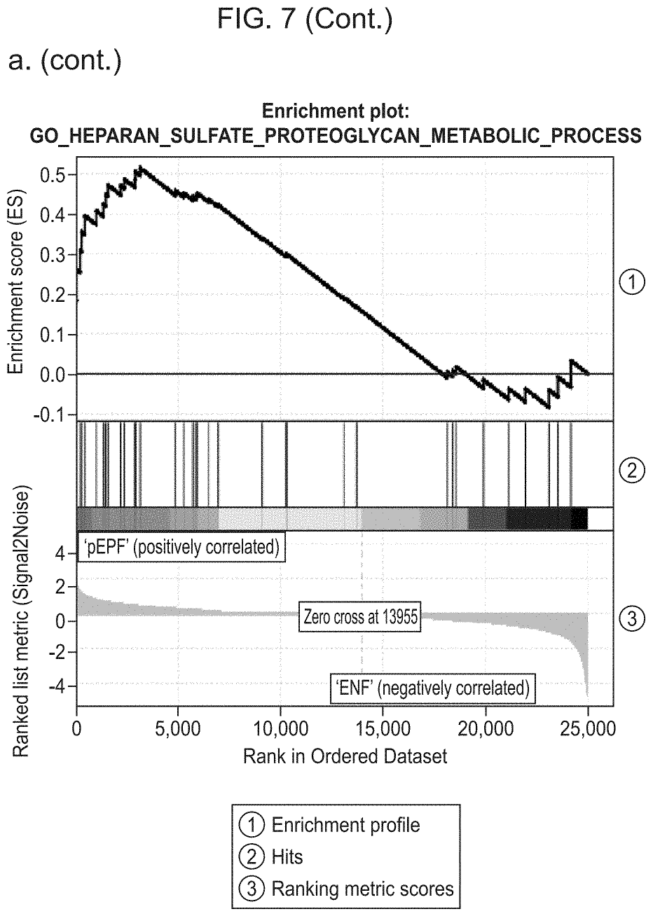

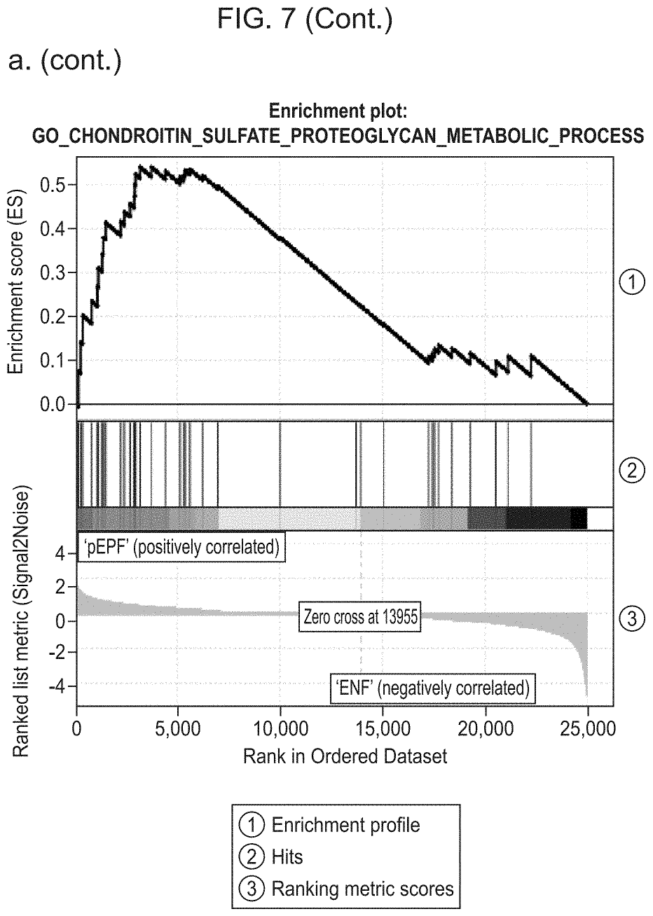

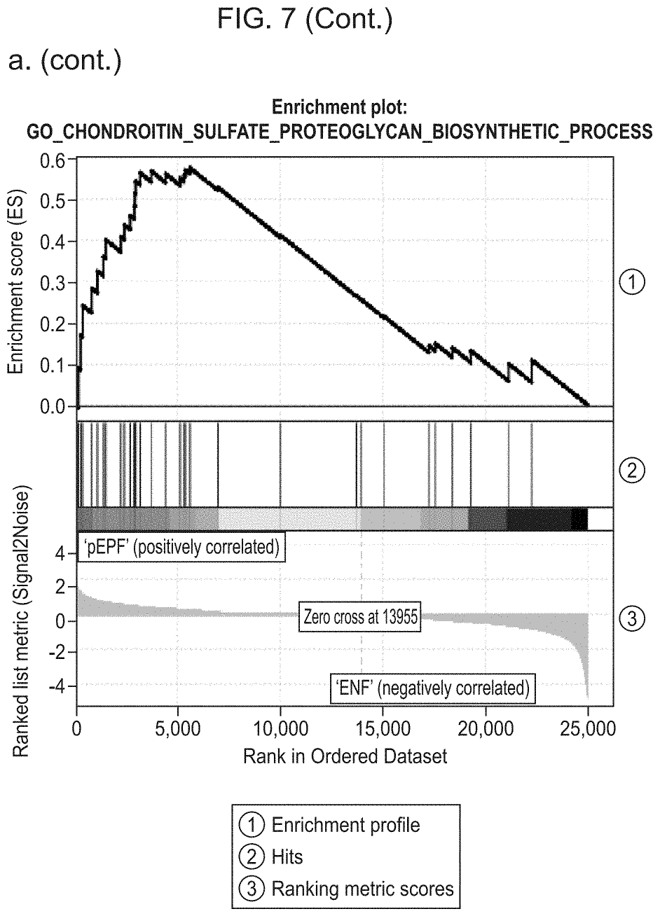

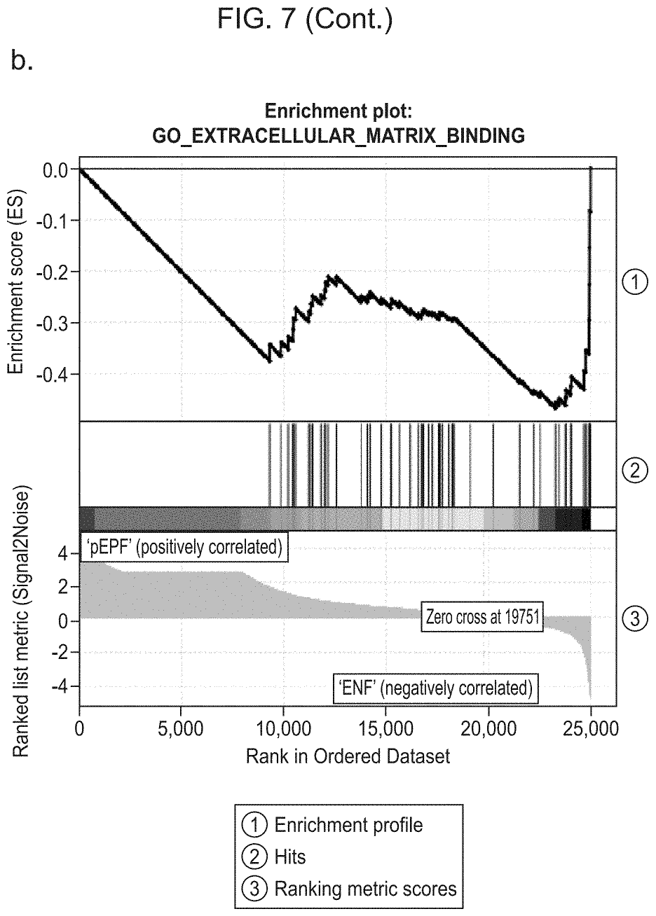

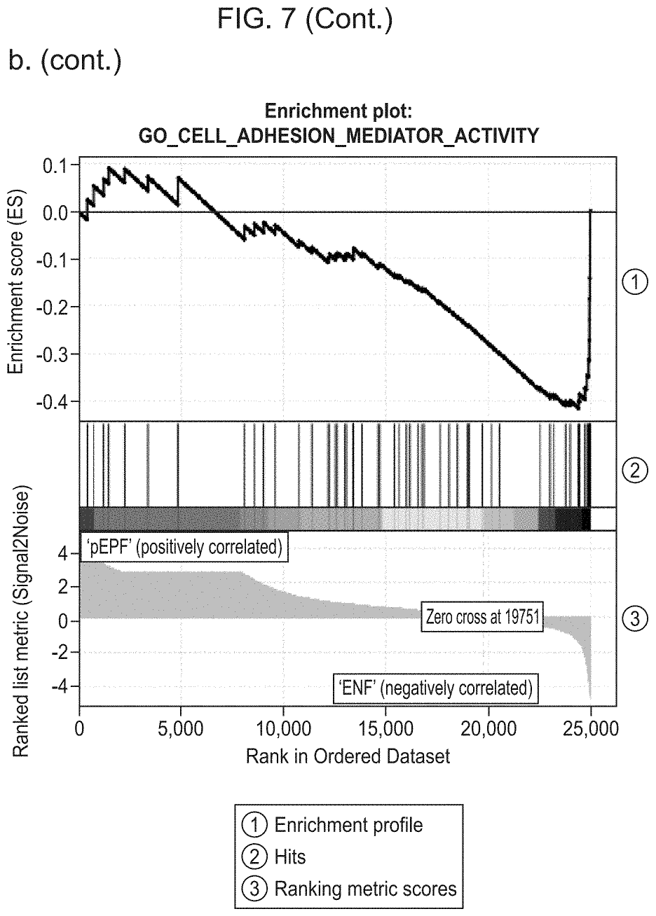

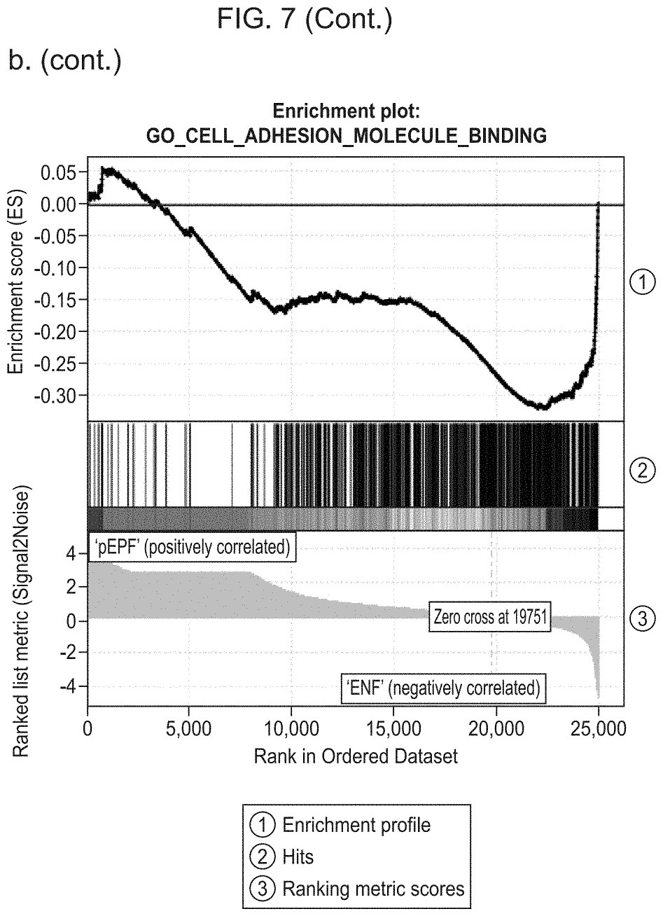

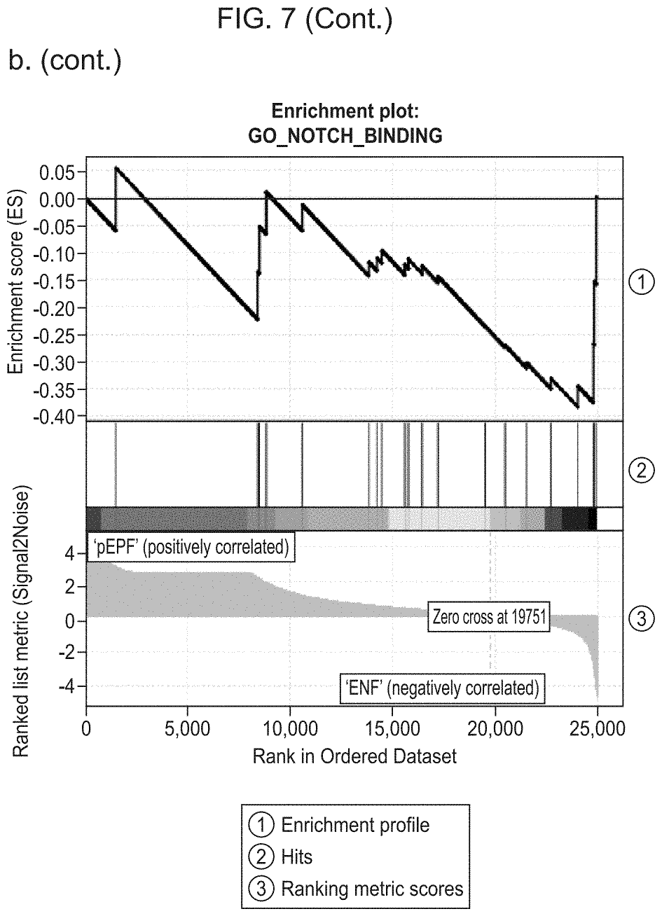

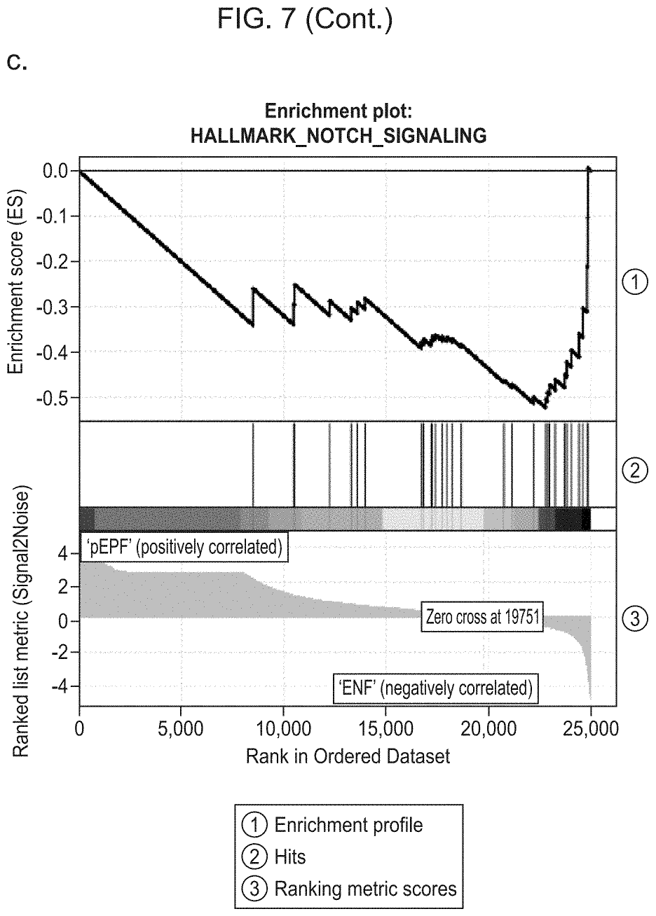

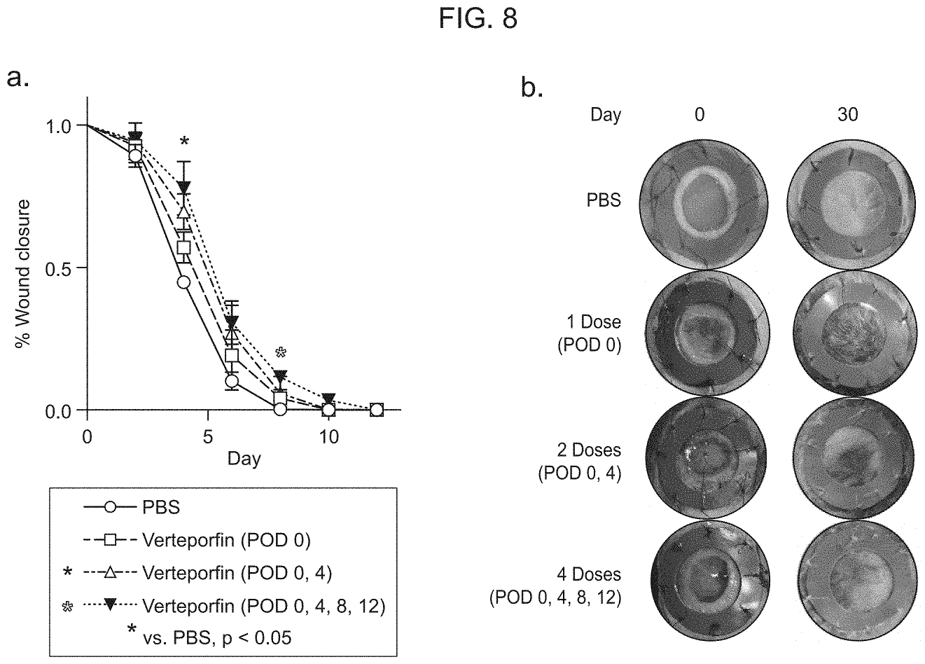

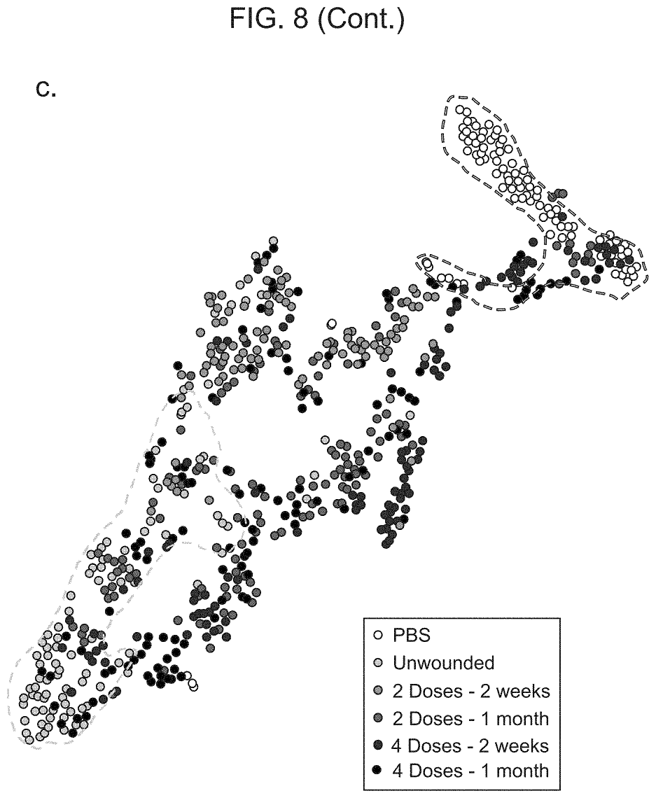

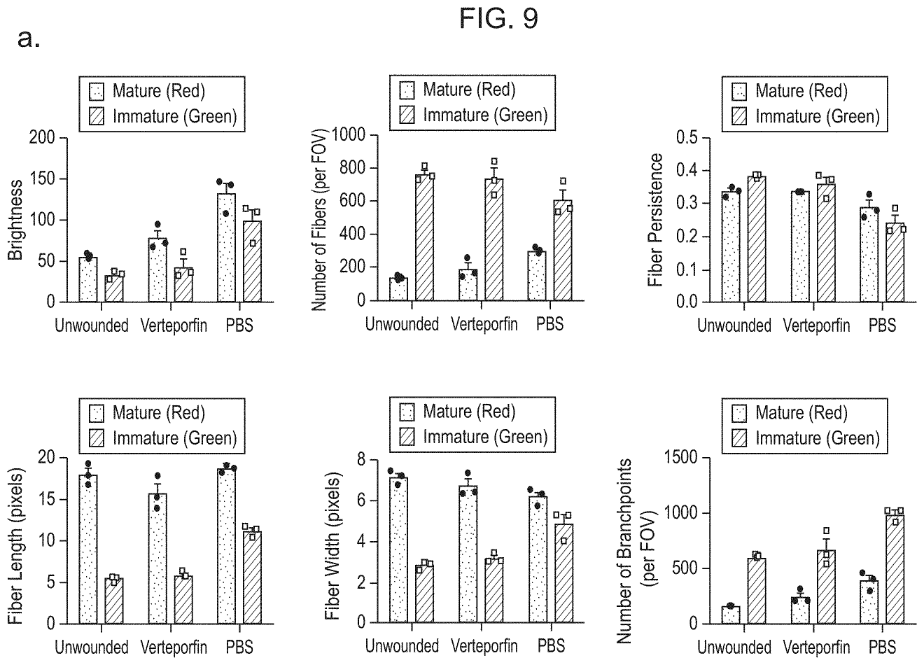

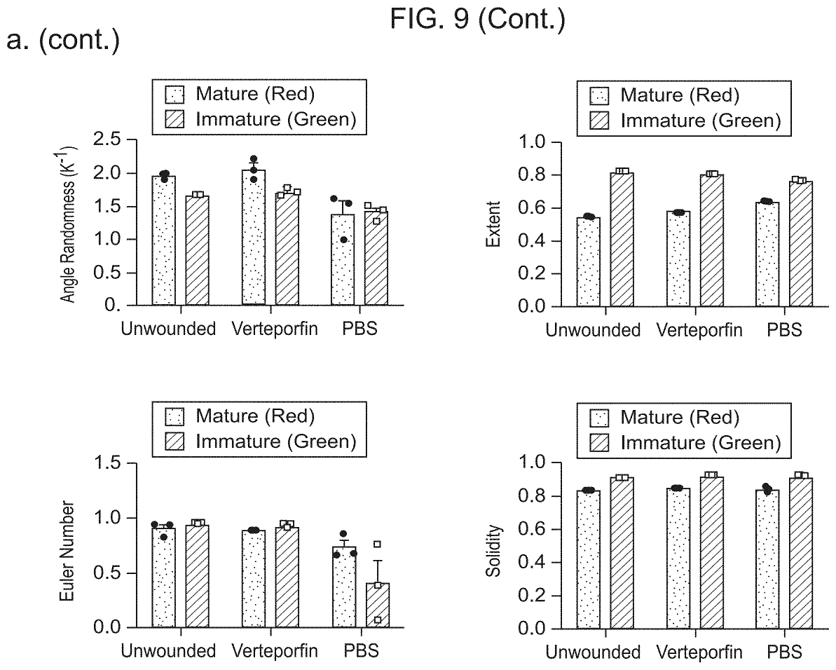

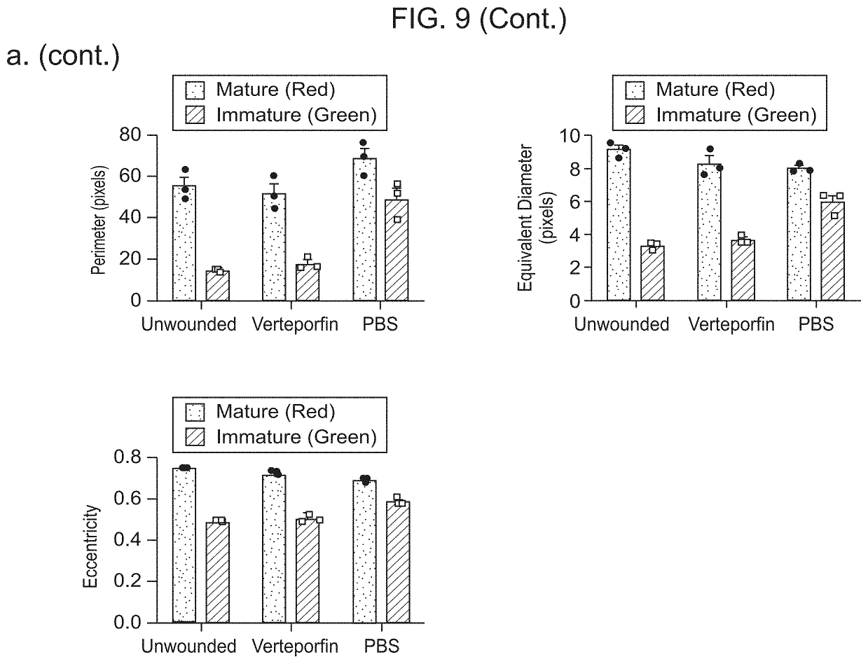

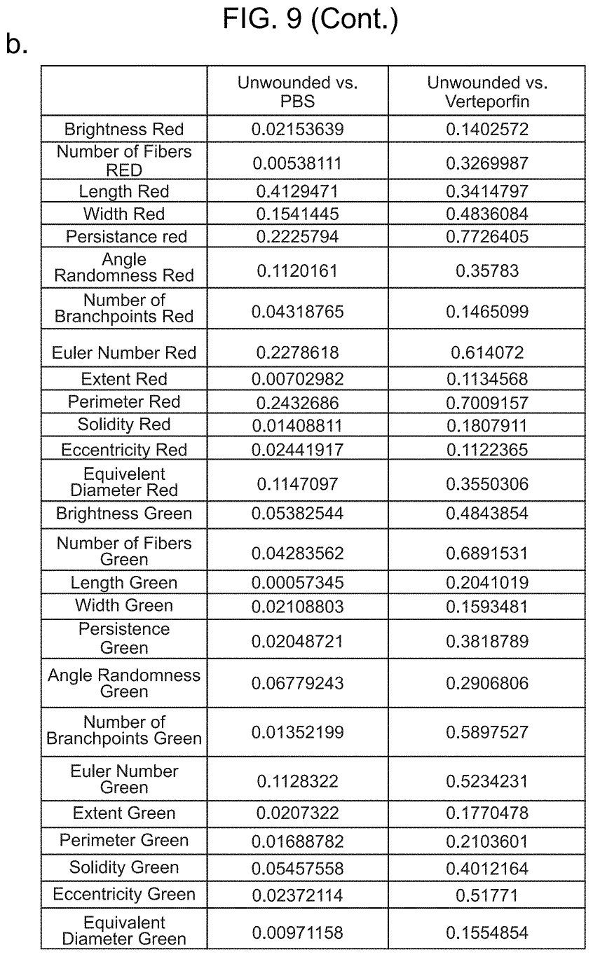

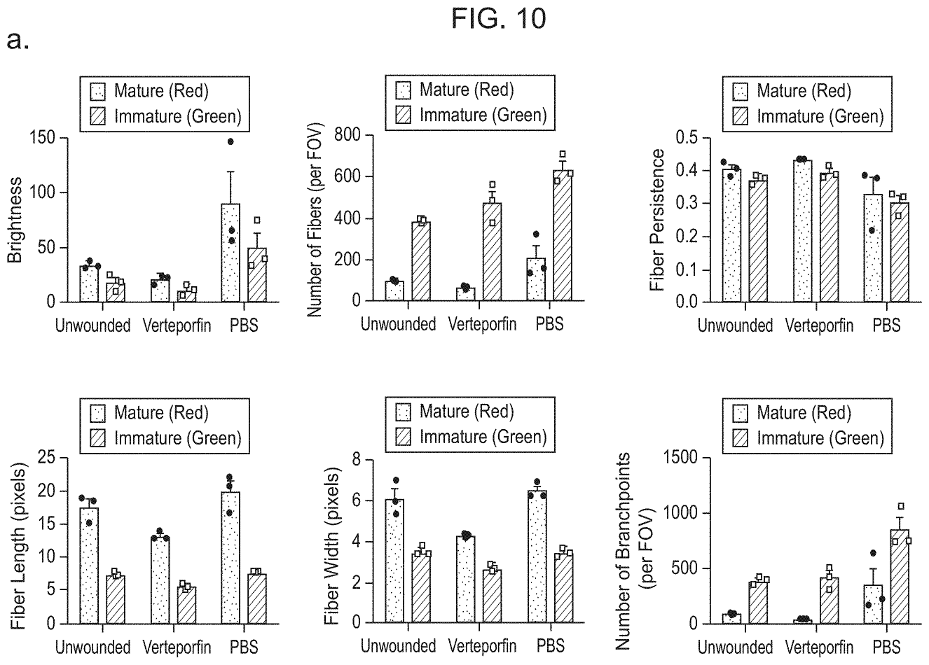

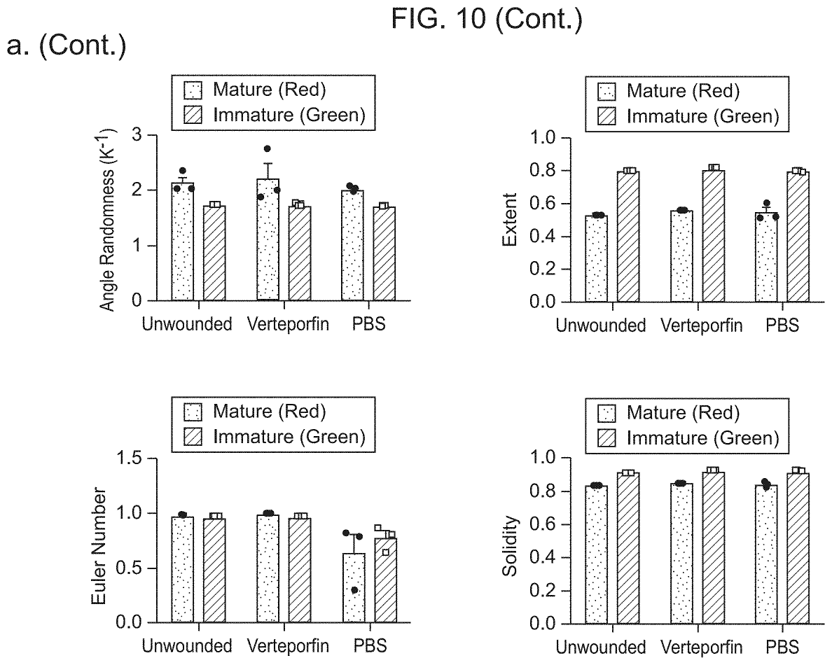

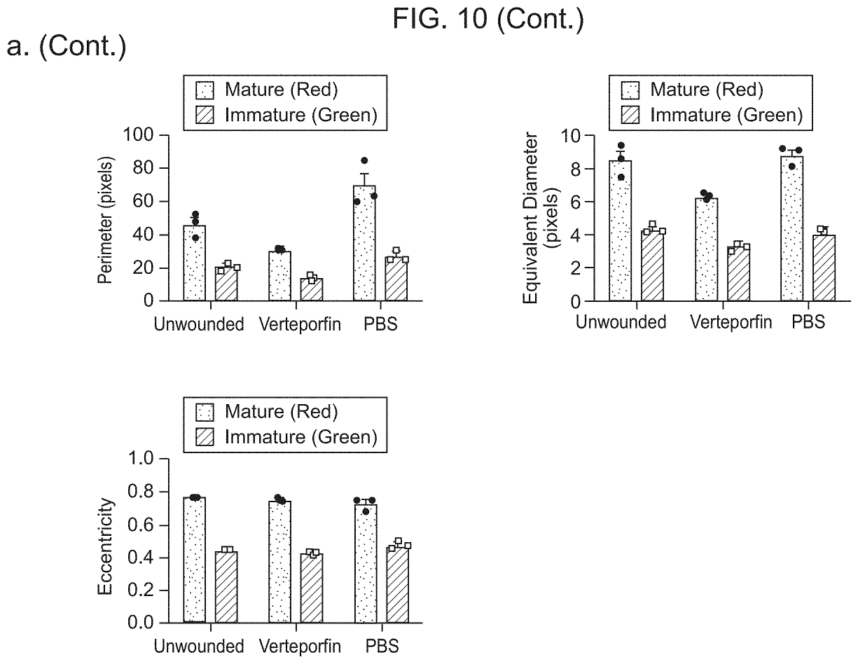

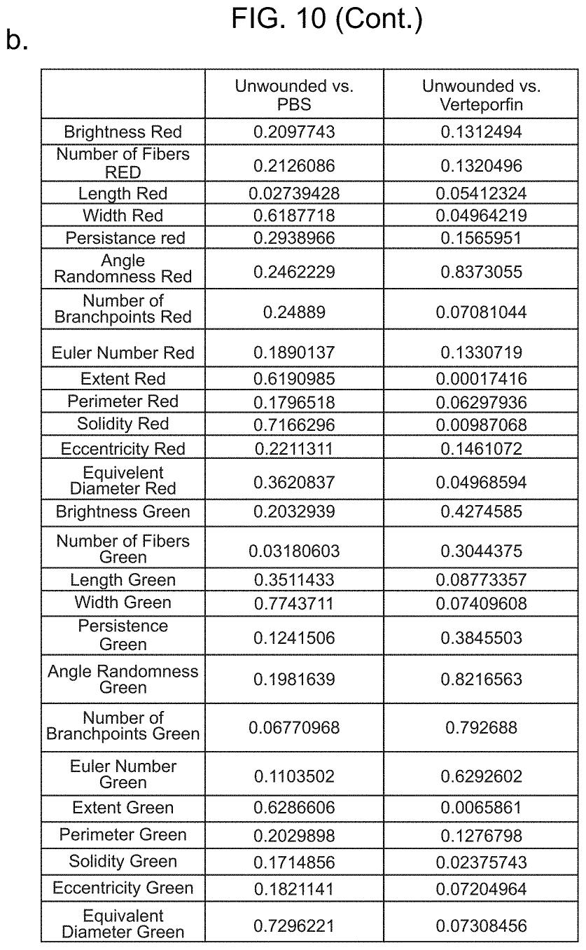

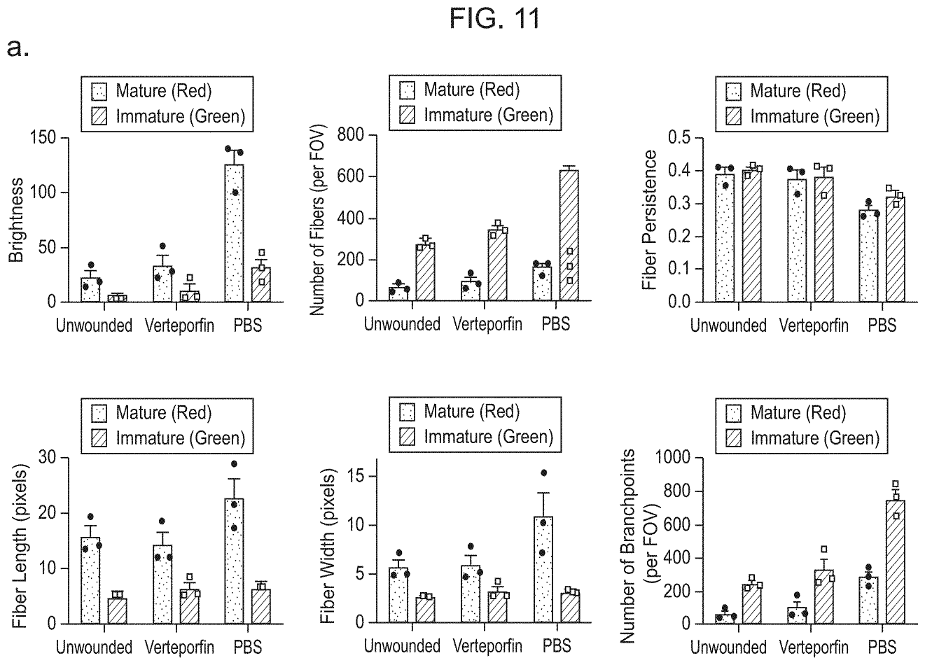

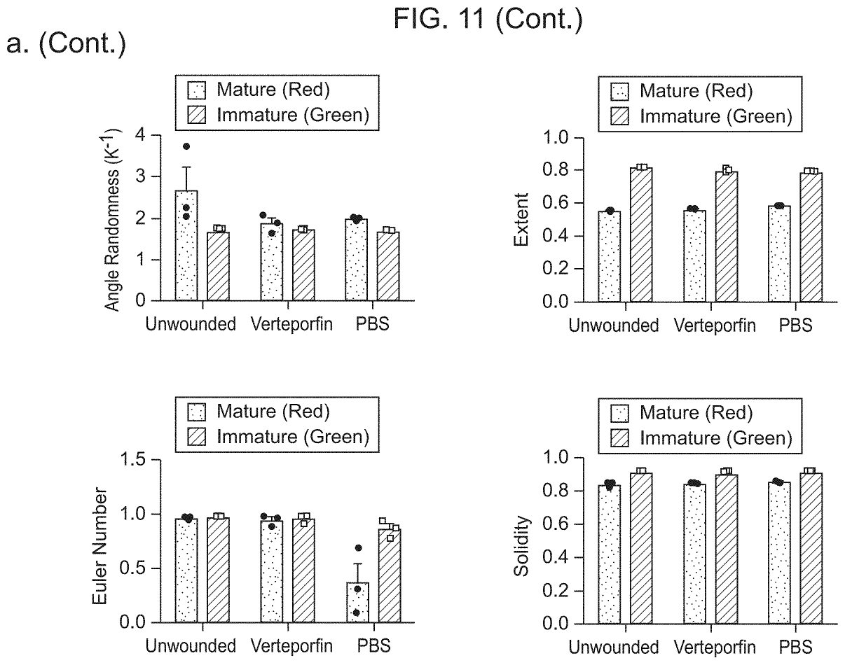

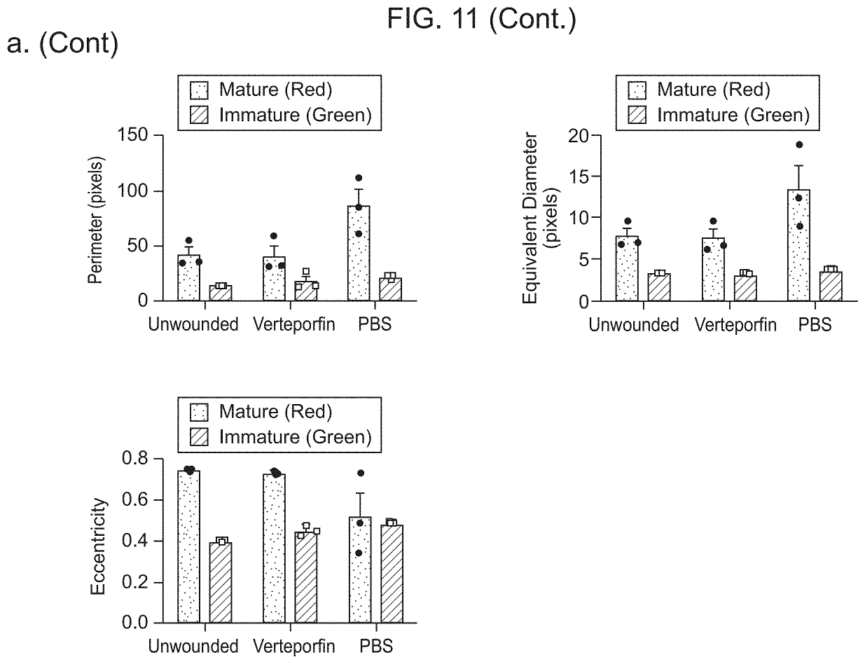

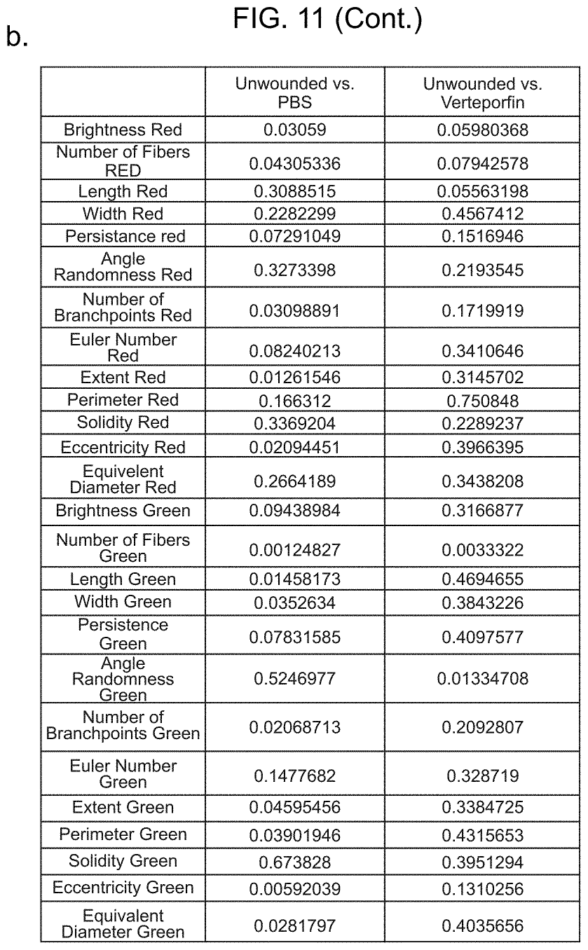

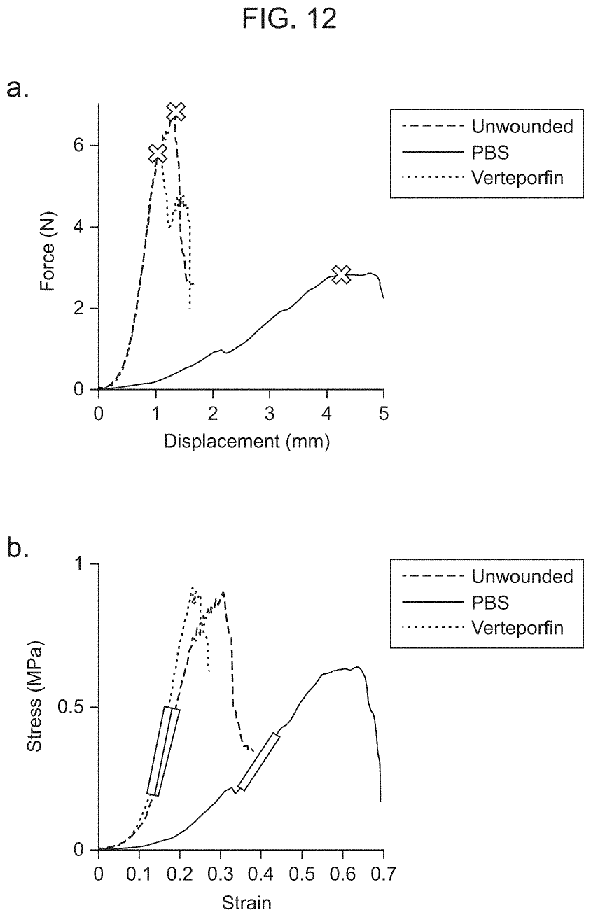

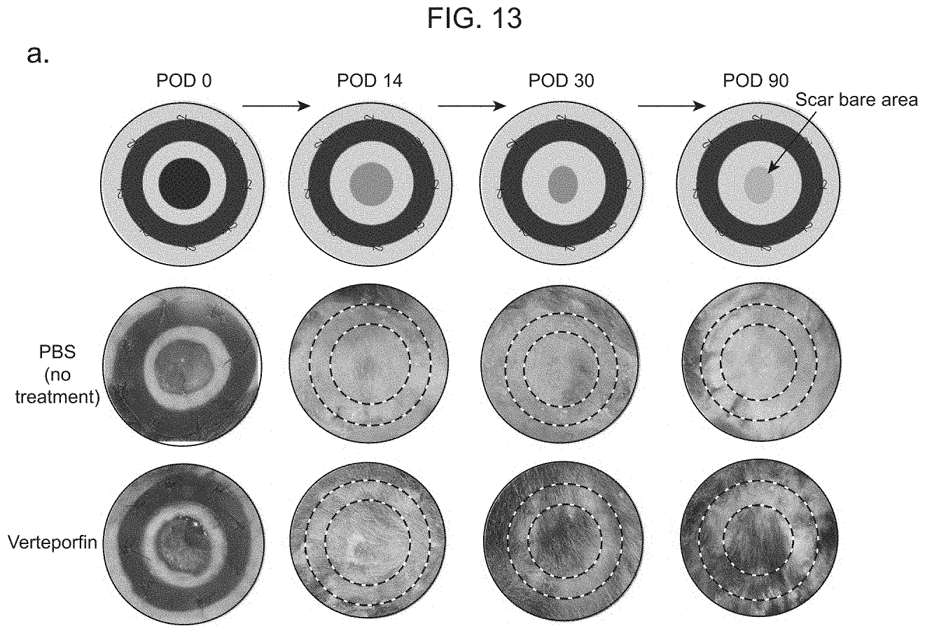

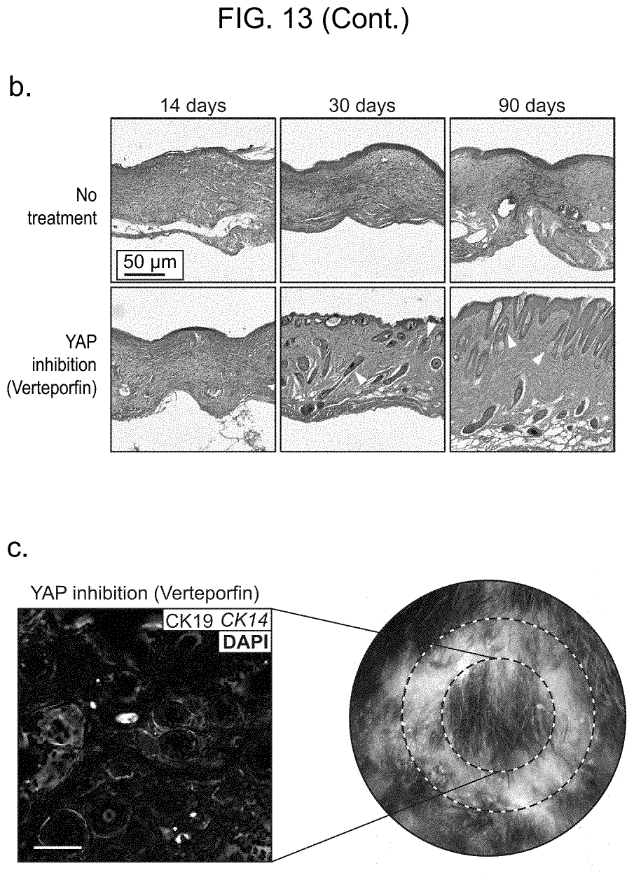

20 mm-long linear wounds were produced on the dorsum of P60 En-1^{Cre-ERT};Ai6 mice and then closed with sutures. On POD 4, a loading device (constructed from 22 mm expansion screws and Luhr plate supports) was secured over each wound with adhesive and simple interrupted sutures. For mice receiving mechanotransduction signaling inhibitor, 30 μL of Verteporfin (1 mg/mL) was injected along the suture line both at POD 0 and POD 4; PBS was injected into wounds for vehicle controls. Device tension was increased by distracting expansion 2 mm every 2 days for 10 days total. Mice with unexpanded devices served as sham surgery controls. On POD 14, wounds were harvested and processed for histology to characterize the effects of increased wound tension on activation of ENFs to scarring pEPFs. In FIG. 2, G-I, a minimum of 4 mice was used for each experimental group.

### Histology and Immunofluorescent Staining

Tissues were fixed in 2% paraformaldehyde for 16 h at 4° C. Samples were prepared for embedding by soaking in 30% sucrose in PBS for 1 week at 4° C. Samples were then removed from the sucrose solution, and tissue blocks were prepared by embedding in Tissue Tek U.C.T. (Sakura Finetek) under dry ice to achieve rapid freezing. Frozen blocks were mounted on a Thermo Scientific CryoStar NX70 cryostat, and 10 μm-thick sections were transferred to SuperfrostfPlus adhesive slides (Fisher). For hematoxylin and eosin staining, standard protocols were used with no modifications. For immunofluorescent staining, slides were blocked for 1 hr with Power Block (Biogenex) prior to addition of the following primary antibodies: Abeam ab34710 (anti-collagen type I), Abeam ab28340 (anti-CD26), Invitrogen MA5-15915 (anti-Dlk1), Abeam ab51317 (anti-Scat), Abeam ab5694 (anti-α-SMA), Santa Cruz Biotechnology sc-101199 (anti-YAP), Abeam ab7800 (anti-CK14), and Abeam ab52625 (anti-CK19). Slides were then incubated for 1 h with Alexa Fluor 568 or Alexa Fluor 647-conjugated anti-rabbit, anti-rat, or anti-mouse antibodies (Invitrogen). Finally, slides were mounted in Fluoromount-G mounting solution with DAPI (Thermo Fisher). Brightfield images were acquired with a Leica DMI4000B microscope, while fluorescent images were acquired with a Leica DM6000 SP5 upright confocal microscope.

### Imaris Pixel Co-localization Analysis

Confocal z-stacks were analyzed using Imaris 8.1.2 software (Bitplane). The surfaces of collagen-I immunofluorescence and of the transplanted ENFs or pEPFs were first reconstructed in three-dimensions. Next, the percent of surface contact between collagen I and the transplanted fibroblasts was determined by the colocalization module. Each dot in FIG. 1, D represents the average contact calculated from the immunofluorescence histology of one wound.

### Bulk RNA Sequencing

Total RNA was harvested by lysing cells in Trizol reagent (Invitrogen), RNA extraction and library preparation was performed by the Stanford Functional Genomics facility using standard Qiagen kits and protocols. Directional RNA-Sal libraries were analyzed with an Agilent Bioanalyzer to ensure successful library creation, and then sequenced with the Illumina HiSeq 4000 System (2×75 bp, 150 cycles), Paired-end reads were mapped to the mouse genome reference sequence mm10 using the STAR aligner. Differential gene transcription analysis was achieved in Matlab 2019a using a negative binomial model. It is common practice to normalize read counts by the total number of reads and the length of each transcript, yielding reads per kilobase mapped (RPKM) values. However, such analysis may be skewed towards a few highly expressed genes that dominate the total lane count. Thus, instead, counts were normalized by a size factor, calculated by taking the median of the ratios of observed counts to those of a pseudo-reference sample (whose counts are the geometric means of each gene across all samples). For hypothesis testing of differential gene transcription, the read counts were modeled according to a negative binomial distribution, with the variance considered as the sum of the shot noise term and a locally regressed non-parametric smooth function of the mean. P values were then adjusted by the Benjamini-Hochberg statistical method to account for the multiple testing problem, and counts were considered significantly different at a threshold of 0.00005 for in vitro studies and 0.01 for in vivo studies. Raw RNA-seq data can be accessed at the following Github repository: https://github.com/shamikmascharak/Mascharak-et-al-ENF.

### Gene Set Enrichment Analysis

Gene Ontology (GO) analysis of significantly up- or down-regulated genes in FIG. 3, E was performed using g.Profiler (https://blit.cs.utee/qprofiler/gost) with a p value cutoff of 0.05. Ranked whole genome enrichment analyses in FIGS. 6 and 7 were performed using GSEA software developed by the Broad Institute with nominal p value and false discovery rate (FDR) cutoffs of 0.01 and 0.25, respectively. All g.Profiler and GSEA results are available in the following Github repository: https://github.com/shamikmascharak/Mascharak-et-al-ENF.

### Quantitative Analysis of Collagen Ultrastructure

For analysis of Picrosirius Red-stained histologic sections, scars and surrounding normal skin from three biologic replicates were randomly imaged at 5 to 10 separate locations each, for a minimum of 20 images per experimental condition. Next, color deconvolution of Picrosirius Red images was performed in ImageJ using the algorithm previously described by Ruifrok et al., (A. C. Ruifrok, D. A. Johnston, Quantification of histochemical staining by color deconvolution. Anal Quant Cytol Histol 23, 291-299 (2001)) wherein each pure stain is characterized by absorbances within three RGB channels (Color 1=[1 0 0], Color 2=[0 1 0], Color 3=[1 1 1]). Ortho-normal transformation of the histology images produced individual images corresponding to each color's individual contribution to the image. Applied to birefringent Picrosirius Red images (green to red color under polarized light depending on packing of fiber bundles), this technique produced deconvoluted red and green images corresponding to mature and immature connective tissue fibers, which were then analyzed independently. Analysis was thus performed purely using extracellular matrix fibers, with no cellular elements included. Noise reduction of deconvoluted fibers was achieved using an adaptive Wiener filter in Matlab 2019a (wiener2 function), which tailors itself to the local image variance within a pre-specified neighborhood (3-by-3 pixels in the current application). The filter preferentially smooths regions with low variance, thereby preserving sharp edges of fibers. Smooth images were then binarized using the im2bw command and processed through erosion and dilation filters with both linear and diamond-shaped structuring elements to select for fiber-shaped objects. Finally, the fiber network was “skeletonized” using the bwmorph command and various parameters of the digitized map (fiber length, width, persistence, alignment, etc.) were measured using the regionprops command. Dimensionality reduction of quantified fiber network properties by 1-distributed stochastic neighbor embedding (t-SNE) was achieved using the default tsne (distance metric specified as Euclidian distance) command in Matlab. A Matlab script containing the fiber quantification pipeline is available at the following Github repository: https://github.com/shamikmascharak/Mascharak-et-al-ENF

### Tensile Strength Testing

Tensile strength tests for unwounded skin (N=7) and PBS (N=5) or Verteporfin-treated (N=4) wounds in P60 C57BL/6 mice were conducted at POD 30 using an Instron 5565 equipped with a 100 N load cell. Dorsal skin was harvested and cut into 4 mm-by-15 mm strips. Tissue strips were then secured using custom grips with the scar positioned equidistant to each grip edge and preloaded to a force of 0.02 N to remove slack before the length of the tissue was measured using digital calipers; the width and thickness of the strips were also re-measured to confirm accurate dimensions. Finally, the skin was subjected to an extension test to failure, defined by a sharp decrease in stress with increasing strain, at a rate of 1%/s. The wound breaking force, or yield force, was determined at the maximal force before the tissue entered plastic deformation and eventual failure. True strain was calculated as the change in length divided by the original gauge length, and true stress was calculated as the force divided by the original cross-sectional area. The Young's Modulus was calculated by taking a least-squares regression of the slope during the linear, elastic portion of the stress-strain curve (R²>0.99).

### B. Results:

### 1 A Fibroblast Subpopulation Activates Engrailed-1 in the Wound Environment

In order to elucidate the response of defined fibroblast lineages to the in vivo wound environment, ENFs (Tomato⁺) and EPFs (GFP) were isolated from the skin of En-1^{Cre};R26^{mTmG} mice, Each fibroblast subtype (ENFs or EPFs) was transplanted intradermally into the dorsum of separate eight-week-old wildtype (non-fluorescent) mice and then the skin within the engrafted region was wounded (Le., the injected area was larger than the wounded area, such that a ring of injected cells remained around the wound margins). The wound was then allowed to heal, and upon complete healing (at 14 days), the healed wounds (scars) and surrounding unwounded skin were harvested (experimental schematic in FIG. 1, A). Histological analysis was performed to examine the phenotype of engrafted cells, both those in the unwounded skin and those that had migrated into the wound.

Within the unwounded skin, all engrafted fibroblasts (EPFs and ENFs) demonstrated quiescent morphology with linearly elongated cell bodies (FIG. 1, B top row). As expected, EPF-engrafted unwounded skin contained only GFP⁺ cells (Le., EPFs; FIG. 1, B top left), and ENF-engrafted unwounded skin contained only Tomato+ cells (i.e., ENFs) with no GFP⁺ cells, which would indicate En-1 activation (leading to Ore driven recombination of the mT/mG fluorescent reporter; FIG. 1, B top right). In contrast to EPFs in unwounded skin, engrafted EPFs within scars exhibited activated, migratory morphology with multiple extended cellular processes (FIG. 1, B bottom left), consistent with prior reports of wound EPF phenotype. (Y. Rinkevich et al., Skin fibrosis. Identification and isolation of a dermal lineage with intrinsic fibrogenic potential. Science 348, aaa2151 (2015)) Strikingly, wounds engrafted with ENFs were found to contain numerous GFP⁺ cells with activated morphology similar to that of wound-engrafted EPFs (FIG. 1, B bottom right), indicating that transplanted ENFs had activated En-1 expression to become postnatally-derived EPFs (pEPFs) in response to the wound environment. To confirm the fibrotic phenotype of these pEPFs, immunofluorescent staining was performed for type I collagen (col-I; FIG. 1, C). Pixel colocalization analysis confirmed that pEPFs had significantly greater overlap with col-I than did wound-engrafted ENFs (FIG. 1, D), indicating increased collagen production specifically from the cells that had activated En-1.

These engraftment results strongly suggested that ENFs activated En-1 within the wound environment. However, it was important to rule out the possibility that the sorted ENFs contained a small number of contaminating EPFs, and that these disproportionately proliferated in the wound to give rise to the GFP⁺ cells observed in ENF-transplanted wounds. To definitively confirm that postnatal ENFs activate En-1 expression during adult wound healing, an En-1^{Cre-ERT};Ai6 transgenic mouse model was generated. In this model, En-1^{Cre}-driven recombination of the fluorescent Ai6 reporter (leading to GFP expression) can only occur following induction with tamoxifen. Thus, tracing of En-1 expression can be temporally controlled. In order to robustly demonstrate postnatal ENF-to-EPF transition, systemic tamoxifen induction of En-1^{Cre-ERT};Ai6 mice was performed prior to wounding, such that any GFP⁺ fibroblasts in the scar would necessarily represent EPFs that arose via En-1 activation during wound healing. The scars and surrounding unwounded tissue were harvested upon complete wound healing (day 14; experimental schematic in FIG. 1, E, FACS isolation strategy in FIGS. 5, A and 5, B). In tamoxifen-induced En-1^{Cre-ERT};Ai6 mice, only rare GFP⁺ cells were noted in unwounded skin (FIG. 1, F top left). This finding suggested that Ore recombination does not occur to a significant extent outside of the wound, supporting the notion that En-1 expression is activated specifically in response to the wound environment. In marked contrast to unwounded skin, in healed wounds, roughly 40% of fibroblasts were GFP⁺ (FIG. 1, F bottom left, FIG. 5, C), These data corroborated the findings of En-1 activation in wound-engrafted ENFs and suggested that postnatal ENF-to-EPF transition generates a substantial fraction of scar-producing EPFs (schematic in FIG. 1, G).

While these data demonstrated that engrafted ENFs can postnatally activate En-1 (i.e., become pEPFs) in response to the wound environment, they did not implicate a specific subset of ENFs capable of this behavior. Emerging literature has indicated that unwounded skin fibroblasts comprise multiple anatomically distinct subpopulations with unique surface markers. (R. R. Driskell et al., Distinct fibroblast lineages determine dermal architecture in skin development and repair. Nature 504, 277-281 (2013), R. R. Driskell, F. M. Watt, Understanding fibroblast heterogeneity in the skin. Trends Cell Biol 25, 92-99 (2015)) The goal was to identify whether ENFs corresponding to these different subpopulations might exhibit distinct phenotypes in the wound context and, in particular, whether the ability to activate En-1 might be specific to any ENF subpopulation. Using flow cytometry, dorsal dermal fibroblasts were obtained from En-1^{Cre};Ai6 mice, Fibroblasts (Lin⁻; see Methods for details) were first sorted into En-1 lineage-positive cells (GFP⁺) and En-1 lineage-negative cells (no intrinsic fluorescence). Based on previously reported surface markers, (R. R. Driskell et al., Distinct fibroblast lineages determine dermal architecture in skin development and repair. Nature 504, 277-281 (2013), R. R. Driskell, F. M. Watt. Understanding fibroblast heterogeneity in the skin. Trends Cell Biol 25, 92-99 (2015)) ENFs were then further sorted into papillary dermal (CD26⁺Sca1⁻), reticular dermal (Dlk1⁺Sca1⁻), and hypodermal (Dlk1^{+/-}Sca1⁺) subtractions (experimental schematic in FIG. 1, H, FACS isolation strategy in FIGS. 5, D and 5, E).

Similar to prior reports, (R. R. Driskell et al., Distinct fibroblast lineages determine dermal architecture in skin development and repair. Nature 504, 277-281 (2013)) papillary, reticular, and hypodermal fibroblasts comprised 19%, 12%, and 52% of PDGFRa⁺ ENFs (FIG. 5, F left panel); when instead compared on the basis of lineage negativity, the three populations were more evenly distributed (FIG. 5, F right panel). However, it was observed that a significant fraction of ENFs did not express PDGFRa (FIG. 5, E‡). Therefore, this marker was not included in the sorting strategy. The papillary, reticular, and hypodermal ENF subpopulations were then separately engrafted into R26^{mTmG} (Tomato⁺) mice prior to wounding, as described above for bulk ENFs (FIG. 1, H). In scars with engrafted papillary dermal or hypodermal ENFs, no GFP⁺ cells were observed, indicating a lack of En-1 activation in these ENF subpopulations (FIG. 1, I lett and right panels). However, numerous GFP⁺ cells were observed in scars containing transplanted reticular dermal ENFs. (FIG. 1, l middle panel, white arrowheads). These findings suggested that reticular dermal (Dlk1⁺Sca1⁺) ENFs are the primary ENF subpopulation capable of postnatal En-1 activation in response to wounding.

When Dlk1 expression was examined in the skin and wounds of tamoxifen-induced En-1^{Cre-ERT};Ai6 mice, Dlk1 expression in unwounded skin was confined to the deep dermis, consistent with previous reports of Dlk1 as a reticular (deep) dermal fibroblast marker (FIG. 1, F top right). (R. R Driskell et al., Distinct fibroblast lineages determine dermal architecture in skin development and repair. Nature 504, 277-281 (2013), R. R. Driskell, F. M. Watt, Understanding fibroblast heterogeneity in the skin. Trends Cell Biol 25, 92-99 (2015)) In scars, Dlk1 expression was observed throughout all dermal layers (FIG. 1, F bottom). Notably, Dlk1⁺ ENFs were found in close association with chains of pEPFs (GFP⁺) (FIG. 1, F bottom right, white arrowheads). These data further supported the concept that Dlk1⁺Sca1⁻ reticular ENFs activate En-1 in response to the wound environment to contribute to scarring.

### 2. Postnatal Engrailed-1 Activation is Mechanoresponsive

Fibroblasts interact with their environment through cell surface receptors called integrins. These transmembrane receptors couple to focal adhesion kinase (FAK) to convert mechanical cues into transcriptional changes via Rho and Rho-associated protein kinase (ROCK) signaling. (P. P. Provenzano, P. J. Keely, Mechanical signaling through the cytoskeleton regulates cell proliferation by coordinated focal adhesion and Rho GTPase signaling. Journal of cell science 124, 1195-1205 (2011)) Publications have demonstrated that this mechanical signaling, or mechanotransduction, pathway modulates wound-resident cells in scarring. (L. A. Barnes et al., Mechanical Forces in Cutaneous Wound Healing: Emerging Therapies to Minimize Scar Formation. Adv Wound Care (New Rochelle) 7, 47-56 (2018); S. Aarabi et al., Mechanical load initiates hypertrophic scar formation through decreased cellular apoptosis. FASEB journal: official publication of the Federation of American Societies for Experimental Biology 21, 3250-3261 (2007); V. W. Wong et al., Focal adhesion kinase links mechanical force to skin fibrosis via inflammatory signaling. Nat Med 18, 148-152 (2011)) Fibroblasts in particular are known to be highly sensitive to mechanical stimuli. Physically increasing tension across a wound causes resident fibroblasts to increase expression of pro-fibrotic genes such as collagens and TGF-β;(S. Aarabi et al., Mechanical load initiates hypertrophic scar formation through decreased cellular apoptosis. FASEB journal : official publication of the Federation of American Societies for Experimental Biology 21, 3250-3261 (2007)) conversely, offloading wound tension reliably leads to reduced scarring. (M. T. Longaker et al., A randomized controlled trial of the embrace advanced scar therapy device to reduce incisional scar formation. Plast Reconstr Surg 134, 536-546 (2014)) Given the established contribution of substrate mechanics to scarring and fibroblast phenotype, it was reasoned that wound-related mechanical cues may promote the activation of ENFs to fibrotic pEPFs.

To test this hypothesis, ENFs isolated from En-1^{Cre;}R≈^{mTmG} mice were cultured in vitro in one of three mechanical environments: (1) collagen-coated tissue culture plastic (TCPS; high stiffness); (2) TCPS with ROCK inhibitor Y-27632 (high stiffness with blocked stiffness sensing); and (3) 3D collagen hydrogels (low stiffness) (experimental schematic in FIG. 2, A). After 14 days in culture, ENFs grown on stiff substrate (TOPS) had largely activated En-1 expression, as evidenced by their conversion to GFP⁺ EPFs (FIG. 2, B left column and 2, C, green circles). In contrast, ENFs grown in a low-stiffness environment (soft hydrogel) remained largely CFP⁻, indicating minimal En-1 activation (FIG. 2, B right column and 2, C, blue triangles). A similar lack of En-1 activation was observed when cellular mechanotransduction signaling was blocked using a ROCK inhibitor (FIG. 2, B middle column and 2, C, red squares), mimicking the effects of a lower-stiffness substrate.

In order to determine whether En-1 activation in response to mechanical tension varied between different ENF subpopulations, ENFs were anatomically fractionated from En-1^{Cre};Ai6 mice as in the engraftment studies. Then each population was cultured on TCPS, with or without ROCK inhibitor Y-27632 (experimental schematic in FIG. 2, D). Papillary dermal and hypodermal ENFs showed little to no En-1 activation on the stiff substrate (FIG. 2, E left and right columns). In contrast, reticular dermal (Dlk1⁺) ENFs showed near-complete conversion to GFP⁺pEPFs after 14 days (FIG. 2, E top middle column), consistent with an earlier finding that pEPF generation after ENF engraftment and wounding was unique to Dlk1⁻E ENFs (FIG. 1, I). En-1 activation in Dlk1⁺ ENFs was blocked with the addition of ROCK inhibitor (FIG. 2, E bottom middle). These data suggested that Dlk1⁺ reticular ENFs activate En-1 expression in response to mechanical cues, which are signaled via canonical mechanotransduction pathways involving ROCK (FIG. 2, F).

Then, whether mechanical tension similarly promoted ENF-to-EPF transition in vivo was assessed. In order to test this hypothesis, incisional wounds were created on the dorsa of tamoxifen-induced En-1^{Cre-ERT};Ai6 mice and these wounds were subjected to mechanical loading following a previously established protocol (S. Aarabi et al., Mechanical load initiates hypertrophic scar formation through decreased cellular apoptosis. FASEB journal: official publication of the Federation of American Societies for Experimental Biology 21, 3250-3261 (2007)) Distraction (expansion) devices were affixed over each wound and expanded over 10 days, allowing for tension across the wound to be increased in a controlled fashion throughout the course of healing (FIG. 2, G left panel schematic). Grossly, mechanically loaded scars appeared thickened and raised compared to control wounds (for which distraction devices were applied but not expanded) (FIG. 2, G middle and left photographs). Consistent with this grossly hypertrophic appearance, histology of mechanically loaded scars showed greater expression of YAP and α-SMA (FIG. 2, H middle and left columns), consistent with increased mechanotransduction signaling. Importantly, increasing wound tension was also found to significantly increase the number of pEPFs (GFP⁺) and YAP+ cells in wounds (FIG. 2, H middle column and 2, I).

The observations that ENFs activate En-1 and adopt a fibrotic phenotype in response to mechanical tension, and that ROCK inhibition blocks postnatal En-1 activation, strongly suggested that the postnatal ENF-to-EPF transition is dependent on canonical mechanotransduction signaling (e.g., FAK, ROCK). In response to mechanical stimulation, YAP (the final transcriptional effector of mechanotransduction) is known to translocate to the nucleus to activate proliferation- and migration-related genes. (T. Panciera, L. Azzolin, M. Cordenonsi, S. Piccolo, Mechanobiology of YAP and TAZ in physiology and disease. Nature reviews. Molecular cell biology 18, 758-770 (2017), F. Liu et Mechanosignaling through YAP and TAZ drives fibroblast activation and fibrosis. Am J Physiol Lung Cell Mol Physiol 308, L344-357 (2015)) Recently, it was shown that YAP activates lung fibroblasts into a feedback loop that sustains pulmonary fibrosis. (F. Liu et al., Mechanosignaling through YAP and TAZ drives fibroblast activation and fibrosis. Am J Physiol Lung Cell Mol Physiol 308, L344-357 (2015)) It was hypothesized that YAP may similarly promote fibrosis in skin scarring by driving ENFs' transition to the fibrotic pEPF phenotype.

To assess this hypothesis, distracted dorsal wounds were treated with Verteporfin, a chemical inhibitor of YAP mechanotransduction signaling (FIG. 2, F). Treatment with Verteporfin mitigated the effects of increased wound tension: mechanically loaded wounds that were treated with Verteporfin grossly resembled control (non-mechanically loaded) wounds (FIG. 2, G right photograph) and contained significantly fewer pEPFs compared to mechanically loaded, non-Verteporfin treated wounds (FIG. 2, H right column, FIG. 2, I top panel). Immunofluorescent staining confirmed decreased YAP and a-SMA expression in Verteporfin-treated compared to untreated wounds (FIG. 2, H right column), with significantly fewer YAP+ cells in Verteporfin-treated wounds (FIG. 2, I bottom panel). Collectively, these results demonstrated that mechanical tension drives ENF-to-EPF transition in vivo during wound healing.

### 3. Postnatally-Derived EPFs Recapitulate Embryonically-Derived EPF Signatures

In order to ascertain whether in vitro En-1 activation involved a shift toward a fibrotic transcriptomic profile, bulk ENFs were isolated from En-1^{Cre};R26^{mTmG} mice and grew these cells on TCPS for 2 days (at which point ENFs remain single cells), 7 days (when ENFs form colonies), or 14 days (when ENFs activate En-1) (experimental schematic in FIG. 3, A). Cultured cells were then subjected to bulk RNA-seq analysis.

Hierarchical clustering of 920 genes that were significantly up- or down-regulated after 14 days in culture (>4-fold increase or 4-fold decrease, respectively, compared to initial 2 day timepoint; FIGS. 3, B and 3, C) revealed a transcriptional shift over time (FIGS. 3, B and 3, D). Gene Ontology (GO) annotation of genes upregulated at 14 days (g.Profiler) included multiple terms related to ECM deposition (FIG. 3, E top panel), suggesting pro-fibrotic changes in stiffness-activated ENFs. In contrast, genes related to muscle development were more highly expressed in ENFs at early time points but became downregulated over time in culture (FIG. 3, E bottom panel). Similarly, Gene Set Enrichment Analysis (GSEA, Broad Institute) of ranked whole genomes showed increased representation of terms related to ECM production and deposition, epithelial-mesenchymal transition, and loss of “muscle identity” terms after 14 days (FIG. 6). These findings are consistent with reports that native ENFs express muscle-related genes; (Y. Rinkevich et al., Skin fibrosis. Identification and isolation of a dermal lineage with intrinsic fibrogenic potential. Science 348, aaa2151 (2015)) this phenotype may be lost as mechanically-activated ENFs shift toward a more fibrotic phenotype. Interestingly, the highest Dlk1 expression was observed at 7 days (the “colony stage”; FIG. 3, F, red box). This finding suggests that the Dlk1⁺ ENF subpopulation disproportionately expanded in culture by 7 days (resulting in increased representation of DIM expression in the bulk sample). Consistent with the g. Profiler and GSEA findings, multiple ECM genes (e.g., collagens, fibronectin) were upregulated at 14 days (FIG. 3, F, green box), following activation of En-1 expression.

Next, to assess if postnatal En-1 activation was dependent on mechanotransduction signaling, ENFs cultured on TCPS were grown in the presence of Verteporfin. After 14 days in culture, treated cells were subjected to RNA-seq. Mechanotransduction blockade attenuated the transcriptomic shift observed in untreated cells (FIG. 3, B, purple box). GO term analysis in g. Profiler demonstrated decreased enrichment of ECM-related terms and relatively higher muscle development-related terms, indicating that these cells more closely retained their native ENF identity (FIG. 3, E). Consistent with this pattern, visualization of all RNA-seq data by principal component analysis (RCA) showed that ENFs treated with Verteporfin for 14 days more closely resembled untreated cells that had only been in culture for 2 days (FIG. 3, D, purple cluster). Verteporfin-treated ENFs also exhibited reduced expression of various ECM genes (FIG. 3, F, purple box), suggesting that YAP inhibition blocked generation of fibrogenic pEPFs.

To study transcriptional changes that occur during in viva ENF-to-EPF transition, five fibroblast populations were isolated from tamoxifen-induced En-1^{Cre-ERT};Ai6 mice and analyzed by bulk RNA-seq: pEPFs (GFP⁺) from wounded skin; eEPFs (GFP⁻CD26⁺) from unwounded and wounded skin; and ENFs (GFP⁻CD26⁻) from unwounded and wounded skin (experimental schematic in FIG. 3, G). Hierarchical clustering (FIG. 3, H) of 1,138 differentially expressed genes after wounding (FIG. 3, I) revealed that pEPFs clustered more closely with eEPFs than with ENFs. A similar pattern was observed upon comparing transcriptomic profiles by RCA (FIG. 3, J). Both postnatally- and embryonically-derived EPFs (pEPFs and eEPFs, respectively) showed increased expression of fibrosis-related genes in response to wounding, including Dpp4 (CD26), despite the fact that pEPFs were sorted based only on GFP expression and not specifically gated for CD26 expression (FIG. 3, K left panel, FIG. 5, B). On the other hand, ENFs showed increased expression of several YAP signaling-related genes (Notch ligands Jag1, Dll1), (A. Totaro, M. Castellan, D. Di Biagio, S. Piccolo, Crosstalk between YAP/TAZ and Notch Signaling. Trends Cell Biol 28, 560-573 (2018)) particularly after wounding, suggesting that they are mechanoresponsive to the wound environment (FIG. 3, K middle and right panels). Supporting these findings, GSEA of ranked whole genomes revealed that scar ENFs enriched for terms related to ECM adhesion and Notch signaling, while postnatal EPFs (which putatively arise from mechanically activated ENFs) enriched for terms related to ECM production and deposition (FIG. 7). Finally, transcriptional activity of various genes previously reported to differentiate ENFs (FIG. 3, L left) and eEPFs (FIG. 3, L right) were compared. (Y. Ainkevich et al., Skin fibrosis. Identification and isolation of a dermal lineage with intrinsic fibrogenic potential. Science 348, aaa2151 (2015)) Once again, pEPFs were found to diverge from ENFs, exhibiting a gene expression profile more closely resembling that of eEPFs (FIG. 3, L, green boxes). Thus, postnatal En-1 activation in mechanosensitive ENFs, both in vitro and in vivo, was accompanied by acquisition of a pro-fibrotic transcriptional profile similar to that of embryonically-derived EPFs.

### 4. Modulating YAP Signaling Promotes Regenerative ENF-Mediated Wound Healing

Given that En-1 activation was associated with adoption of a pro-fibrotic phenotype, and that YAP inhibition prevented En-1 activation in vitro, whether YAP inhibition could also block En-1 activation in vivo to reduce scarring in a mouse wounding model was assessed. Adult En-1^{Cre};R26^{mTmG} mice were wounded, and the POD 0 wounds were treated by injecting the wound base with either PBS (control) or Verteporfin (1 mg/mL). Importantly, YAP inhibition at this dosing regimen did not significantly affect wound closure rate (FIG. 8, A, red circles vs. blue squares). Wounds were harvested for gross and histological examination at POD 14, 30, or 90. As expected, control wounds healed in a typical scarring fashion (FIG. 4, A middle row). Even after 90 days, the wound site remained bare, forming a distinct region of pale scar tissue in which no hair regrowth occurred (FIG. 4, A middle row, right image). In dramatic contrast, wounds that had been treated with Verteporfin had substantial hair growth within the healed wound by 30 days, and by 90 days the healed wound was grossly indistinguishable from unwounded skin (FIG. 4, A bottom row). This was a striking result, as a hallmark of adult mammalian (scarring) wound healing is a complete lack of regeneration of secondary appendages (e.g., hair follicles, sweat glands), as exemplified by the bare area that remained following control wound healing. However, the gross findings suggested that Verteporfin-treated wounds exhibited regenerative healing. Thus, the goal was to further probe the extent to which healed Verteporfin-treated wounds resembled healthy, unwounded skin, rather than scar tissue.

Consistent with their respective gross appearances, hematoxylin and eosin (H&E) staining showed that control wounds contained dense, parallel collagen bundles with no secondary elements (FIG. 4, B top row), while Verteporfin-treated wounds demonstrated reduced fibrosis and increased cellularity by 2 weeks and contained numerous structures morphologically resembling hair follicles or sweat glands by 1 and 3 months (FIG. 4, B bottom row, white arrows). Confirming true regeneration of secondary elements, Verteporfin-treated wounds exhibited positive IF staining of these appendages for cytokeratins 14 and 19 (CK14 and CK19, markers of hair follicle and sweat gland identity, respectively; FIG. 4, C top) and positive lipid staining using Oil Red O (FIG. 4, C bottom), indicating presence of functional regenerated sebaceous glands.

Consistent with the findings that inhibition of mechanotransduction signaling reduced ENF-to-EPF transition in vitro (FIGS. 2, B and 2, C), it was observed that whereas control wounds contained abundant EPFs (GFP⁺; FIG. 4, D upper left) throughout the dermis after 14 days, Verteporfin-treated wounds contained almost exclusively ENFs (Tomato⁺; FIG. 4, D lower left). Control wounds at 14 days demonstrated strong staining for col-I and minimal staining for fibronectin (Fn; FIG. 4, D top right), consistent with typical scar ECM. However, Verteporfin-treated wounds at this timepoint had substantially reduced col-I staining and comparatively stronger staining for fibronectin (previously reported to be the dominant, provisional matrix protein deposited by ENFs; (D. Jiang et al., Two succeeding fibroblastic lineages drive dermal development and the transition from regeneration to scarring. Nat Cell Biol 20, 422-431 (2018)) FIG. 4, D bottom right), suggesting that YAP inhibition blocked the transition of ENFs into pro-fibrotic pEPFs in response to wounding. At 30 days, Verteporfin-treated wounds again contained significantly fewer EPFs and decreased staining for CD26, relative to control wounds (FIG. 4, E left). IF staining of control wounds demonstrated Dlk1 expression limited to the deep dermis (FIG. 4, E top left, red) and chains of YAP⁺ cells migrating into the scar (FIG. 4, E top right). In contrast, in Verteporfin-treated wounds, Dlk1⁺ cells were present throughout the dermis (FIG. 4, E bottom left) and chains of YAP⁺ cells were markedly shorter (FIG. 4, bottom right). Collectively, these results suggested that conversion of Dlk⁺ ENFs to pEPFs was disrupted by mechanotransduction inhibition, supporting the in vitro findings (FIG. 2, E middle column). When healed wounds were examined after three months of healing, control wounds had widespread GFP expression (indicating EPFs and EPF-derived matrix) with numerous YAP⁺ cells (FIG. 4, F top row and far right panel). These fibroblasts stained positively for α-SMA⁺, consistent with a pro-fibrotic myofibroblast phenotype (FIG. 4, F top). Verteporfin-treated wounds, in contrast, continued to exhibit dramatically reduced numbers of EPFs with rare YAP⁺ cells and virtually no α-SMA⁺ cells (FIG. 4, F bottom row and far right panel). Overall, these data strongly indicate that blocking ENF mechanical activation in wounds leads to regenerative, ENF-driven repair.

While gross and histologic assessment strongly suggested that YAP inhibition reduced scarring, visual analysis of such specimens is subjective and qualitative, and thus prone to bias. (K. W. Eva, G. R. Norman, Heuristics and biases-a biased perspective on clinical reasoning. Med Educ 39, 870-872 (2005), A. Tversky, D. Kahnernan, Judgment under Uncertainty: Heuristics and Biases. Science 185, 1124-1131 (1974)) Further, while Verteporfin-treated wounds appeared grossly similar to unwounded skin (FIG. 4, A bottom row), it was important to confirm that Verteporfin truly resulted in skin regeneration without fibrosis, rather than simply causing hair growth that visually obscured the scar. To overcome these challenges, a novel machine learning algorithm recently reported to quantitatively assess connective tissue and fibrosis based on standard histology stains was employed. (S. Mascharak et al., Automated machine learning analysis of connective tissue networks in acute and chronic skin fibroses (manuscript submitted). (2019)) Briefly, images of Picrosirius Red-stained tissue were color-deconvoluted to isolate ECM fibers from cell bodies and nuclei. Fiber components were image-processed to reduce noise, then binarized to produce a digital map of thousands of fibers and branchpoints. A panel of individual (e.g., length, width) and group (e.g., packing, alignment) fiber properties was then measured to quantitatively profile ECM features.

Verteporfin- and control-treated specimens were stained with Picrosirius Red and subjected to this analysis. Across multiple metrics (fiber length, width, branching, etc.), POD 14 Verteporfin-treated wounds were quantitatively distinct from control (PBS) wounds and instead were comparable to unwounded skin (FIGS. 9, A and 9, B). RCA of the connective tissue parameters confirmed that YAP inhibition in wounds yielded ECM resembling unwounded skin after 14 days, as demonstrated by largely overlapping clusters for Verteporfin-treated wounds and unwounded skin (FIG. 4, G, panel i). Similar analyses after 30 and 90 days of healing showed increasing overlap between these two groups at 30 days and complete overlap at 90 days (FIG. 4, G, panel ii and iii; FIGS. 10 and 11), indicating that tissue treated with Verteporfin at the time of wounding continued to remodel in a regenerative fashion over time. Thus, the quantitative analysis confirmed that YAP inhibition significantly reduced scarring and promoted skin regeneration in a mouse model of wound healing.

Given that Verteporfin appeared to have continued effects over the course of wound healing, the effects of administering multiple doses of Verteporfin throughout the wound repair process was assessed. Wounds treated with two doses of Verteporfin (POD 0 and 4) exhibited wound closure rates, gross appearance, and ECM features comparable to those of wounds treated with single Verteporfin dose (POD 0) (FIG. 8, A-C). However, when Verteporfin dosage was further increased to four treatments (POD 0, 4, 8, and 12), wound closure was delayed (FIG. 8, A), hair regrowth was grossly reduced (FIG. 8, B), and ECM features diverged from those of unwounded skin (FIG. 8, C). Thus, Verteporfin affected scarring in a dose-dependent manner, with detrimental effects observed upon excessive dosing.

Notably, despite the fact that typical scars are characterized by excess collagen, they are significantly weaker than unwounded skin and will regain at most 80% of the strength of healthy skin, (C. D. Marshall et al., Cutaneous Scarring: Basic Science, Current Treatments, and Future Directions. Adv Wound Care (New Rochelle) 7, 29-45 (2018)) due to their inferior collagen organization. The findings up to this point showed that Verteporfin treatment yielded healed wounds that grossly and histologically resembled unwounded skin and, importantly, possessed ECM ultrastructural properties that did not significantly differ from those of unwounded skin. It was also critical to determine whether this regeneration of skin architecture resulted in functional recovery of normal skin's mechanical robustness. In order to characterize the physical properties of Verteporfin-treated wounds, tensile testing was performed on unwounded skin and PBS- or Verteporfin-treated wounds after 30 days of healing. Consistent with scars' decreased structural integrity, healed control wounds had significantly decreased tensile strength compared to unwounded skin (FIG. 4, H, green vs. red), as measured by wound breaking force and Young's modulus, In contrast, the tensile strength of Verteporfin-treated wounds did not significantly differ from that of unwounded skin (FIG. 4, H, green vs. blue), strongly supporting a restoration of normal skin strength consistent with the regenerative ECM features of these wounds (representative force-displacement and stress-strain curves in FIG. 12).

### C. Discussion:

Fibroblasts are a heterogeneous cell population, consisting of multiple subpopulations with distinct roles and behaviors. (Y. Rinkevich et aL, Skin fibrosis. Identification and isolation of a dermal lineage with intrinsic fibrogenic potential. Science 348, aaa2151 (2015); R. R. Driskell et al., Distinct fibroblast lineages determine dermal architecture in skin development and repair. Nature 504, 277-281 (2013); R. R. Driskell, F. M. Watt, Understanding: fibroblast heterogeneity in the skin. Trends Cell Biol 25, 92-99 (2015); D. Jiang et al., Two succeeding fibroblastic lineages drive dermal development and the transition from regeneration to scarring. Nat Cell Biol 20, 422-431 (2018); E. Marsh, D. G. Gonzalez, E. A. Lathrop, J. Boucher, V. Greco, Positional Stability and Membrane Occupancy Define Skin Fibroblast Homeostasis In Vivo. Cell 175, 1620-1633 e1613 (2018); M. C. Salzer et al., Identity Noise and Adipogenic Traits Characterize Dermal Fibroblast Aging. Cell 175, 1575-1590 e1522 (2018); B. A. Shook et al., Myofibroblast proliferation and heterogeneity are supported by macrophages during skin repair. Science 362, (2018); T. Tabib, C. Morse, T. Wang, W. Chen, R. Lafyatis, SFRP2/DPP4 and FMO1/LSP1 Define Major Fibroblast Populations in Human Skin. J Invest Dermatol 138, 802-810 (2018); M. D. Lynch, F. M. Watt, Fibroblast heterogeneity: implications for human disease. The Journal of clinical investigation 128, 26-35 (2018); C. Philippeos et al., Spatial and Single-Cell Transcriptional Profiling Identifies Functionally Distinct Human Dermal Fibroblast Subpopulations. J Invest Dermatol 138, 811-825 (2018); T. Leavitt et aL, Prrx1 lineage fibroblasts have fibrogenic potential in the ventral dermis. (manuscript submitted), (2019)). Wounding activates a subset of dermal fibroblasts to exhibit contractile properties and exuberant ECM production, (I. A. Darby, T. D. Hewitson, Fibroblast differentiation in wound healing and fibrosis. Int Rev Cytol 257, 143-179 (2007); I. A, Darby, B. Laverdet, F. Bonte, A. Desmouliere, Fibroblasts and myofibroblasts in wound healing. Clin Cosmet lnvestig Dermatol 7, 301-311 (2014); B. Hinz, Formation and function of the myofibroblast during tissue repair. J Invest Dermatol 127, 526-537 (2007); B. Hinz et al., The myofibroblast: one function, multiple origins. Am J Pathol 170, 1807-1816 (2007)) leading to the formation of a fibrotic scar. A dermal fibroblast subpopulation defined by embryonic expression of En-1 (eEPFs) that is responsible for deposition of fibrotic scar tissue in the dorsal skin was previously identified, (Y. Rinkevich et al., Skin fibrosis. Identification and isolation of a dermal lineage with intrinsic fibrogenic potential. Science 348, aaa2151 (2015)) a finding that opened up the field of fibroblast heterogeneity in wound repair. However, the role of En-1 lineage-negative fibroblasts (ENFs) in postnatal wound healing has been minimally studied. Here, it is shown for the first time that ENFs activate En-1 in response to mechanical cues within the wound environment and contribute to scar formation as postnatally-derived EPFs (pEPFs).

Recent work has categorized adult unwounded mouse skin fibroblasts into papillary, reticular, and hypodermal subpopulations based on surface marker expression. (R. R. Driskell et al., Distinct fibroblast lineages determine dermal architecture in skin development and repair. Nature 504, 277-281 (2013), R. R. Driskell, F. M. Watt, Understanding fibroblast heterogeneity in the skin. Trends Cell Biol 25, 92-99 (2015)) While these subdivisions are based on anatomical location, they may also confer distinct phenotypes and, in particular, differing fibrogenic potential. By studying the in vitro and in vivo behavior of anatomically-fractionated ENFs, Dlk⁺ Sca1⁻ reticular ENFs are identified as the predominant mechanosensitive cell type capable of postnatal En-1 activation. Other groups have reported subsets of α-SMA⁺CD26⁺ wound myofibroblasts arising from both En-1 and Dlk-2-lineages.(B. A. Shook et al., Myofibroblast proliferation and heterogeneity are supported by macrophages during skin repair. Science 362, (2018), C. F. Guerrero-Juarez et al., Single-cell analysis reveals fibroblast heterogeneity and myeloid-derived adipocyte progenitors in murine skin wounds. Nature communications 10, 650 (2019)) The findings support the importance of these En-1 and Dlk-1 fibroblast lineages in wound healing and, further, suggest that mechanical forces may serve to bridge these two lineages (i.e., activate Dlk-1⁺ ENF to pEPF), thus explaining their shared contribution to postnatal scar formation.

The contribution of physical tension to scarring has long been recognized by surgeons, who classically incise along relaxed skin tension lines to reduce wound tension, facilitating healing with reduced scarring. It has been shown that either physically offloading tension or chemically blocking cellular mechanotransduction (via FAK inhibition) significantly reduces scar burden. (V. W. Wong et al., Focal adhesion kinase links mechanical force to skin fibrosis via inflammatory signaling. Nat Med 18, 148-152 (2011), M. T. Longaker et aL, A randomized controlled trial of the embrace advanced scar therapy device to reduce incisional scar formation. Plast Reconstr Surg 134, 536-546 (2014), A. F. Lim et al., The embrace device significantly decreases scarring following scar revision surgery in a randomized controlled trial. Plast Reconstr Surg 133, 398-405 (2014)) However, the specific cell populations involved in the pro-fibrotic response to tension, and their molecular mechanisms of mechanotransduction, remained previously unknown. By precisely delineating how physical stimuli activate Dlk⁺ En-1-negative fibroblasts (ENFs) to contribute to fibrosis, YAP is identified as a promising molecular target to prevent scarring. It has been shown that inhibition of YAP signaling prevents ENF-to-EPF transition during wound healing, thus encouraging ENF-mediated wound repair with decreased fibrosis and regeneration of secondary skin elements (hair follicles, sweat glands, sebaceous glands). Based on the findings, it is hypothesized that YAP inhibition, through targeted modulation of pro-fibrotic pathways in a specific subset of scarring fibroblasts, allows for regenerative wound healing without compromising healing. Preventing the fibrotic wound response permits regenerative repair with recovery of secondary elements over the course of months or longer.

The findings may have implications for scar prevention. Attempts at reducing scarring often entail ablation of cell populations known to be fibrogenic, but this approach could impair or delay wound repair by nonspecifically eliminating cells that are needed for proper healing, As such, the “holy grail” of skin regeneration—as defined by recovery of three features of normal skin: 1) secondary elements, 2) ECM structure, and 3) mechanical strength has not been achieved. It is reported that in skin wounds, reticular dermal ENFs are activated to become pro-fibrotic pEPFs that contribute to scarring. Moreover, this ENF-to-EPF transition is a mechanically-driven process that is dependent on YAP signaling. By blocking ENF-to-EPF transition in wound healing, postnatal healing by ENFs without compromising speed or efficacy in healing is achieved. Most strikingly, skin regeneration in adult mouse wounds is demonstrated as supported by three key findings: 1) regrowth of secondary skin elements; 2) restoration of normal matrix architecture; and 3) recovery of mechanical robustness.

The observation that ENF-mediated wound healing in postnatal life satisfies the above three criteria for regenerative wound repair implies that regeneration may represent a “default” pathway for wound repair, that is later superseded by the emergence of scarring EPFs.

### Example 2

### Use of Verteporfin for Treatment of Alopecia

### A. Materials and Methods

Adult mice were used for cutaneous wound healing experiments in accordance with well-established protocols. Briefly, mice were anesthetized (2% isofluorane), their dorsal hair was removed with depilatory cream, and the dorsal skin was prepped with alcohol wipes. Next, two 6 mm full-thickness circular wounds were placed through the panniculus carnosus on the dorsum of each animal at the same level, roughly 6 mm below the ears and 4 mm lateral to the midline. The wounds were then stented open by 12 mm diameter silicone rings secured around the wound perimeter with glue and 8 simple interrupted Ethilon 6-0 sutures (Ethicon). For mice receiving mechanotransduction inhibitor, 30 μL of Verteporfin (1 mg/mL) was injected locally into the wound base; PBS was injected into wounds for vehicle controls. Post-operative analgesia was accomplished with buprenorphine 0.05 mg/kg every four hours for three doses, and then as indicated. Dressings were changed every other day under anesthesia. All wounds were fully re-epithelialized by post-operative day (POD) 14, at which time the wound and surrounding skin (used as unwounded control) were harvested and processed for histology.

### B. Results & Discussion

In the above mouse model of wound healing—which typically results in formation of a hairless region of scar—it has been found that a single Verteporfin treatment (local injection) immediately following wounding leads to a dramatic increase in hair regrowth. Regeneration of new hair follicles in Verteporfin-treated wounds was observed both grossly (FIG. 13, a) and on histology (FIG. 13, b) and immunohistochemical analysis (FIG. 13, c). In contrast, untreated wounds remain bare areas; with no regrowth of hair follicles even after 3 months of healing, Verteporfin treatment did not delay wound closure.

A method including injection of Verteporfin following micro-injury of a region of alopecia (via Fraxel, microneedling, or other similar approaches) may be used to promote increased hair regrowth in the region. This method does not require grafting of active hair follicles from other regions of skin but could instead encourage true de novo hair folliculogenesis in an otherwise hairless area. Multiple existing therapeutic methods and devices cause low-level, diffuse tissue damage to improve tissue quality. For instance, fractional laser resurfacing treatment (Fraxel) causes microscopic injuries throughout the targeted region of skin, which is purported to induce a favorable wound-like environment to promote tissue regeneration. This approach has the added benefit of disrupting the outer protective layer of the skin (the stratum corneum) to improve penetration and absorption of therapeutic agents delivered topically (e.g., minoxidil or finasteride, topical hair-loss therapies).

Notwithstanding the appended claims, the disclosure is also defined by the following clauses:
1. A method of promoting ENF-mediated healing of a wound in a dermal location of a subject, the method comprising:

administering an effective amount of a YAP inhibitor composition to the wound to modulate mechanical activation of Engrailed-1 lineage-negative fibroblasts (ENFs) in the wound to promote ENF-mediated healing of the wound.
2. The method of Clause 1, wherein the method comprises reducing a transition of ENFs to Engralied-1 lineage-positive fibroblasts (EPFs) in the wound.
3. The method of any of Clauses 1-2, wherein the method comprises preserving an amount of ENFs relative to an amount of EPFs present in the wound.
4. The method of Clause 3, wherein the method comprises increasing the amount of ENFs relative to the amount of EPFs present in the wound compared to an amount of ENFs relative to an amount of EPFs present in a wound not treated with the YAP inhibitor composition.
5. The method of any of Clauses 1-4, wherein the ENF-mediated healing of the wound is completed in an amount of time substantially equal to an amount of time for healing of a wound not treated with the YAP inhibitor composition.
6. The method of any of Clauses 1-5, wherein the administering comprises injecting the composition below a topical dermal location of the subject.
7. The method of any of Clauses 1-6, wherein the YAP inhibitor composition comprises a YAP inhibitor.
8. The method of any of Clauses 1-7, wherein the YAP inhibitor composition consists essentially of a YAP inhibitor.
9. The method of any of Clauses 7-8, wherein the YAP inhibitor is a photosensitizing agent.
10. The method of any of Clauses 7-9, wherein the YAP inhibitor is a benzoporphyrin derivative.
11. The method of any of Clauses 7-10, wherein the YAP inhibitor is verteporfin.
12. The method of any of Clauses 1-11, wherein the ENFs comprise Dlk1+ reticular ENFs.
13. The method of any of Clauses 1-12, wherein the subject is an adult.
14. The method of any of Clauses 1-13, wherein the ENF-mediated healing of the wound comprises regeneration of dermal appendages.
15. The method of Clause 14, wherein the dermal appendages comprise hair follicles, sweat glands, and sebaceous glands.
16. The method of any of Clauses 1-15, wherein the ENF-mediated healing of the wound produces a healed wound comprising improved connective tissue architecture compared to the connective tissue architecture in a healed wound not treated with the YAP inhibitor composition.
17. The method of any of Clauses 1-16, wherein the ENF-mediated healing of the wound produces a healed wound with reduced levels of collagen hyperproliferation compared to levels of collagen hyperproliferation in a healed wound not treated with the YAP inhibitor composition.
18. The method of any of Clauses 1-17, wherein the method further comprises forming the wound.
19. The method of any of Clauses 1-18, wherein the wound is a surgical wound.
20. The method of any of Clauses 1-19, wherein the method produces a healed wound with reduced levels of scarring compared to levels of scarring in a healed wound not treated with the YAP inhibitor composition.
21. The method of any of Clauses 1-20, wherein the method produces a scarless healed wound.
22. The method of any of Clauses 1-21, wherein the method is a method for treating a subject for alopecia.
23. The method of any of Clauses 1-22, wherein the method promotes hair growth.
24. The method of Clause 23, wherein the hair growth comprises generating a new hair follicle.
25. The method of any of Clauses 1-24, wherein the dermal location is hairless.
26. The method of any of Clauses 1-25, wherein the dermal location comprises a scar.
27. The method of any of Clauses 1-26, wherein the dermal location is present on the scalp of the subject.
28. The method of any of Clauses 1-27, wherein the subject has alopecia.
29. The method of any of Clauses 1-28, wherein the wound is a microscopic wound,
30. The method of any of Clauses 1-29, wherein the wound is formed by a microneedle or laser.
31. A method of preventing scarring during healing of a wound in a subject, the method comprising:

forming a wound in a dermal location of a subject, and

administering an effective amount of a YAP inhibitor composition to the wound to modulate mechanical activation of Engrailed-1 lineage-negative fibroblasts (ENFs) in the wound to promote ENE-mediated healing of the wound.
32. The method of Clause 31, wherein the wound is a surgical wound.
33. The method of any of Clauses 31-32, wherein the method produces a healed wound with reduced levels of scarring compared to levels of scarring in a healed wound not treated with the YAP inhibitor composition.
34. The method of any of Clauses 31-33, wherein the method produces a scarless healed wound.
35. The method of any of Clauses 31-34, wherein the ENF-mediated healing of the wound produces a healed wound comprising improved connective tissue architecture compared to the connective tissue architecture in a healed wound not treated with a YAP inhibitor.
36. The method of any of Clauses 31-35, wherein the ENE-mediated healing of the wound produces a healed wound with reduced levels of collagen hyperproliferation compared to levels of collagen hyperproliferation in a healed wound not treated with the YAP inhibitor composition.
37. The method of any of Clauses 31-36, wherein the ENF-mediated healing of the wound is completed in an amount of time substantially equal to an amount of time for healing of a wound not treated with a YAP inhibitor.
38. The method of any of Clauses 31-37, wherein the administering comprises injecting the composition below a topical dermal location.
39. The method of any of Clauses 31-38, wherein the YAP inhibitor composition comprises a YAP inhibitor.
40. The method of any of Clauses 31-39, wherein the YAP inhibitor composition consists essentially of a YAP inhibitor.
41. The method of any of Clauses 39-40, wherein the YAP inhibitor is a photosensitizing agent.
42. The method of any of Clauses 39-41, wherein the YAP inhibitor is a benzoporphyrin derivative.
43. The method of any of Clauses 39-42, wherein the YAP inhibitor is verteporfin.
44. The method of any of Clauses 31-43, wherein the ENFs comprise Dlk+ reticular ENFs.
45. The method of any of Clauses 31-44, wherein the subject is an adult.
46. The method of any of Clauses 31-45, wherein the ENF-mediated healing of the wound comprises regeneration of dermal appendages.
47. The method of Clause 46, wherein the dermal appendages comprise hair follicles, sweat glands, and sebaceous glands.
48. A method of promoting hair growth on a subject, the method comprising:

forming a wound in a dermal location of a subject, and

administering an effective amount of a YAP inhibitor composition to the wound to modulate mechanical activation of Engraiied-i lineage-negative fibroblasts (ENFs) in the wound to promote ENF-mediated healing of the wound.
49. The method of Clause 48, wherein the hair growth comprises generating a new hair follicle.
50. The method of any of Clauses 48-49, wherein the dermal location is hairless.
51. The method of any of Clauses 48-50, wherein the dermal location comprises a scar.
52. The method of any of Clauses 48-51, wherein the dermal location is present on the scalp of the subject.
53. The method of any of Clauses 48-52, wherein the subject has alopecia.
54. The method of any of Clauses 48-53, wherein the subject is an adult.
55. The method of any of Clauses 48-54, wherein the wound is a microscopic wound.
56. The method of any of Clauses 48-55, wherein the wound is formed by a microneedle or laser.
57. The method of any of Clauses 48-56, wherein the administering comprises injecting the composition below a topical dermal location.
58. The method of any of Clauses 48-57, wherein the YAP inhibitor composition comprises a YAP inhibitor.
59. The method of any of Clauses 48-58, wherein the YAP inhibitor composition consists essentially of a YAP inhibitor.
60. The method of any of Clauses 58-59, wherein the YAP inhibitor is a photosensitizing agent.
61. The method of any of Clauses 58-60, wherein the YAP inhibitor is a benzoporphyrin derivative.
62. The method of any of Clauses 58-61, wherein the YAP inhibitor is verteporfin.
63. The method of any of Clauses 48-62, wherein the ENFs comprise Dlk1+ reticular ENFs.
64. The method of any of Clauses 48-63, wherein the subject is an adult.
65. The method of any of Clauses 48-64, wherein the ENP-mediated healing of the wound comprises regeneration of dermal appendages.
66. The method of Clause 65, wherein the dermal appendages comprise hair follicles, sweat glands, and sebaceous glands.
67. A kit comprising:

an amount of a YAP inhibitor composition; and

a tissue disrupting device.
68. The kit of Clause 67, wherein the amount of a YAP inhibitor composition comprises an effective amount of the YAP inhibitor composition for modulating mechanical activation of Engraiied-1 lineage-negative fibroblasts (ENFs) in a wound to promote ENP-mediated healing of the wound.
69. The kit of any of Clauses 67-68, wherein the tissue disrupting device forms a microscopic wound.
70. The kit of any of Clauses 67-69, wherein the tissue disrupting device is a microneedle or laser.
71. The kit of any of Clauses 67-70, wherein the kit further comprises a device for injecting the YAP inhibitor composition below a topical dermal location.
72. The method of any of Clauses 67-71, wherein the YAP inhibitor composition comprises a YAP inhibitor,
73. The method of any of Clauses 67-72, wherein the YAP inhibitor composition consists essentially of a YAP inhibitor.
74. The method of any of Clauses 72-73, wherein the YAP inhibitor is a photosensitizing agent.
75. The method of any of Clauses 72-74, wherein the YAP inhibitor is a benzoporphyrin derivative.
76. The method of any of Clauses 72-75, wherein the YAP inhibitor is verteporfin.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above, Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions.

Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present Invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A method of promoting ENF-mediated healing of a wound in a dermal location of a subject, the method comprising:
administering an effective amount of a YAP inhibitor composition to the wound to modulate mechanical activation of Engrailed-1 lineage-negative fibroblasts (ENFs) in the wound to promote ENF-mediated healing of the wound.

2. The method of claim 1, wherein the method comprises reducing a transition of ENFs to Engrailed-1 lineage-positive fibroblasts (EPFs) in the wound.

3. The method of claim 1, wherein the method comprises preserving an amount of ENFs relative to an amount of EPFs present in the wound.

4. The method of claim 3, wherein the method comprises increasing the amount of ENFs relative to the amount of EPFs present in the wound compared to an amount of ENFs relative to an amount of EPFs present in a wound not treated with the YAP inhibitor composition.

5. The method of claim 1, wherein the ENF-mediated healing of the wound is completed in an amount of time substantially equal to an amount of time for healing of a wound not treated with the YAP inhibitor composition.

6. The method of claim 1, wherein the administering comprises injecting the composition below a topical dermal location of the subject.

7. The method of claim 1, wherein the YAP inhibitor composition comprises a YAP inhibitor.

8. The method of claim 1, wherein the YAP inhibitor composition consists essentially of a YAP inhibitor.

9. The method of claim 7, wherein the YAP inhibitor is a photosensitizing agent.

10. The method of claim 7, wherein the YAP inhibitor is a benzoporphyrin derivative.

11. The method of claim 7, wherein the YAP inhibitor is verteporfin.

12. The method of claim 1, wherein the ENF-mediated healing of the wound comprises regeneration of dermal appendages.

13. The method of claim 12, wherein the dermal appendages comprise hair follicles, sweat glands, and sebaceous glands.

14. A method of promoting hair growth on a subject, the method comprising:
forming a wound in a dermal location of a subject, and
administering an effective amount of a YAP inhibitor composition to the wound to modulate mechanical activation of Engrailed-1 lineage-negative fibroblasts (ENFs) in the wound to promote ENF-mediated healing of the wound.

15. (canceled)

16. The method of claim 14, wherein the hair growth comprises generating a new hair follicle.

17. The method of claim 16, wherein the dermal location is hairless.

18. The method of claim 17, wherein the dermal location is present on the scalp of the subject.
